(19) United States
(12) Patent Application Publication
(10) Pub. No.: US 2016/0324852 A1
(43) Pub. Date: Nov. 10, 2016

(54) COMPOSITIONS AND METHODS OF TREATING A NEURODEGENERATIVE DISEASE

(71) Applicant: Axovant Sciences Ltd., Hamilton (BM)

(72) Inventors: Lawrence Tim FRIEDHOFF, Rivervale, NJ (US); Melissa RHODES, Raleigh, NC (US); Stephen Clement PISCITELLI, Hillsborough, NC (US); Kunal KISHNANI, Flushing, NY (US); Shankar RAMASWAMY, Cincinnati, OH (US); Bryan M. LEWIS, North Brunswick, NJ (US); Geetha RAMASWAMY, New York, NY (US)

(21) Appl. No.: 15/149,053

(22) Filed: May 6, 2016

Related U.S. Application Data
(60) Provisional application No. 62/158,422, filed on May 7, 2015.
(60) Provisional application No. 62/162,060, filed on May 15, 2015.
(60) Provisional application No. 62/162,068, filed on May 15, 2015.
(60) Provisional application No. 62/162,138, filed on May 15, 2015.
(60) Provisional application No. 62/162,193, filed on May 15, 2015.
(60) Provisional application No. 62/165,034, filed on May 21, 2015.
(60) Provisional application No. 62/167,986, filed on May 29, 2015.
(60) Provisional application No. 62/168,246, filed on May 29, 2015.
(60) Provisional application No. 62/169,414, filed on Jun. 1, 2015.
(60) Provisional application No. 62/182,225, filed on Jun. 19, 2015.
(60) Provisional application No. 62/189,089, filed on Jul. 6, 2015.
(60) Provisional application No. 62/191,189, filed on Jul. 10, 2015.
(60) Provisional application No. 62/201,494, filed on Aug. 5, 2015.
(60) Provisional application No. 62/201,513, filed on Aug. 5, 2015.
(60) Provisional application No. 62/239,530, filed on Oct. 9, 2015.
(60) Provisional application No. 62/251,534, filed on Nov. 5, 2015.
(60) Provisional application No. 62/256,349, filed on Nov. 17, 2015.
(60) Provisional application No. 62/261,115, filed on Nov. 30, 2015.
(60) Provisional application No. 62/289,162, filed on Jan. 29, 2016.
(60) Provisional application No. 62/289,643, filed on Feb. 1, 2016.

(51) Int. Cl. A61K 31/496; A61K 45/06; A61K 9/48; A61K 9/00; A61K 31/445

(52) U.S. Cl. CPC A61K 31/496; A61K 9/0053; A61K 31/445; A61K 9/4891; A61K 45/06

(57) ABSTRACT

The present application relates to new uses of 5-HT₆ receptor antagonists, specifically 3-phenylsulfonyl-8-piperazinyl-1yl-quinoline or pharmaceutically acceptable salts, hydrates, solvates, or polymorphs, thereof, and to the combination of 5-HT₆ receptor antagonists, specifically 3-phenylsulfonyl-8-piperazinyl-1yl-quinoline or pharmaceutically acceptable salts, hydrates, solvates, or polymorphs, thereof, with other therapeutic agents for the treatment of a neurodegenerative disease.

### CROSS REFERENCE TO RELATED APPLICATIONS

This application claims the priority benefit under 35 U.S.C. 119(e) of U.S. Provisional Application No. 62/158,422, filed May 7, 2015; U.S. Provisional Application No. 62/162,060, filed May 15, 2015; U.S. Provisional Application No. 62/162,068, filed May 15, 2015; U.S. Provisional Application No. 62/162,138, filed May 15, 2015; U.S. Provisional Application No. 62/162,193, filed May 15, 2015; U.S. Provisional Application No. 62/165,034, filed May 21, 2015; U.S. Provisional Application No. 62/167,986, filed May 29, 2015; U.S. Provisional Application No. 62/168,246, filed May 29, 2015; U.S. Provisional Application No. 62/169,414, filed Jun. 1, 2015; U.S. Provisional Application No. 62/182,225, filed Jun. 19, 2015; U.S. Provisional Application No. 62/189,089, filed Jul. 6, 2015; U.S. Provisional Application No. 62/191,189, filed Jul. 10, 2015; U.S. Provisional Application No. 62/201,494, filed Aug. 5, 2015; U.S. Provisional Application No. 62/201,513, filed Aug. 5, 2015; U.S. Provisional Application No. 62/239,530, filed Oct. 9, 2015; U.S. Provisional Application No. 62/251,534, filed Nov. 5, 2015; U.S. Provisional Application No. 62/256,349, filed Nov. 17, 2015; U.S. Provisional Application No. 62/261,115, filed Nov. 30, 2015; U.S. Provisional Application No. 62/289,162, filed Jan. 29, 2016; and U.S. Provisional Application No. 62/289,643, filed Feb. 1, 2016, the disclosures of which are incorporated by reference in their entirety. This application is also related to co-pending and co-owned U.S. patent application Ser. No. 15/______ filed on May 6, 2015, entitled “Methods of Treating a Neurodegenerative Disease”, (Attorney Docket No. 142956.00901), which is incorporated herein by reference in its entirety.

### SUMMARY

Embodiments herein are directed to compositions comprising: a therapeutically effective amount of 3-phenylsulfonyl-8-piperazinyl-1yl-quinoline or pharmaceutically acceptable salts, hydrates or solvates thereof; a therapeutically effective amount of at least one additional therapeutic agent useful for the treatment of neurodegenerative disease; and at least one pharmaceutically acceptable excipient; wherein the composition is suitable for oral administration.

Some embodiments are directed to methods of treating a neurodegenerative disease in a subject in need thereof comprising administering to said patient a therapeutically effective amount of the composition of claim 1.

Some embodiments are directed to methods of treating a neurodegenerative disease in a subject in need thereof comprising administering to said patient a therapeutically effective amount of 3-phenylsulfonyl-8-piperazinyl-1yl-quinoline or pharmaceutically acceptable salts, hydrates, polymorphs, or solvates thereof.

Some embodiments are directed to pharmaceutical compositions comprising a therapeutically effective amount of 3-phenylsulfonyl-8-piperazinyl-1yl-quinoline or pharmaceutically acceptable salts, hydrates, polymorphs, or solvates thereof and at least one pharmaceutically acceptable excipient; wherein the therapeutically effective amount of 3-phenylsulfonyl-8-piperazinyl-1yl-quinoline comprises at least one polymorphic form of 3-phenylsulfonyl-8-piperazinyl-1yl-quinoline.

### BRIEF DESCRIPTION OF THE DRAWINGS

FIG. 1 is a powder x-ray diffraction pattern of Form I of 3-phenylsulfonyl-8-piperazinyl-1yl-quinoline.

FIG. 2 is a powder x-ray diffraction pattern of Form II of 3-phenylsulfonyl-8-piperazinyl-1yl-quinoline.

FIG. 3 is a powder x-ray diffraction pattern of Form III of 3-phenylsulfonyl-8-piperazinyl-1yl-quinoline.

FIG. 4 is a powder x-ray diffraction pattern of Form IV of 3-phenylsulfonyl-8-piperazinyl-1yl-quinoline.

FIG. 5 is a powder x-ray diffraction pattern of Form V of 3-phenylsulfonyl-8-piperazinyl-1yl-quinoline.

FIG. 6 is a powder x-ray diffraction pattern of Form VI of 3-phenylsulfonyl-8-piperazinyl-1yl-quinoline.

FIG. 7 is a powder x-ray diffraction pattern of Form VII of 3-phenylsulfonyl-8-piperazinyl-1yl-quinoline.

FIG. 8 is a powder x-ray diffraction pattern of Form VIII of 3-phenylsulfonyl-8-piperazinyl-1yl-quinoline.

FIG. 9 is a powder x-ray diffraction pattern of Form IX of 3-phenylsulfonyl-8-piperazinyl-1yl-quinoline.

FIG. 10 shows an exemplary 35 mg 3-phenylsulfonyl-8-piperazinyl-1yl-quinoline/5 mg donepezil capsule formulation. 35 mg 3-phenylsulfonyl-8-piperazinyl-1 yl-quinoline immediate release tablet/5 mg donepezil immediate release tablet taken together in a suitable capsule with or without appropriate excipient backfill. 3-phenylsulfonyl-8-piperazinyl-1yl-quinoline and donepezil tablet may be coated or uncoated, marked or unmarked. Donepezil tablets may be of a standard size produced by an approved generic manufacturer or may be shaped more specifically to fit the capsule. Shape may be round, cylindrical, oval, capsule, or otherwise configured to optimally fit within the volume of the capsule bottom. Tablets will be shaped such that automated capsule filling machinery may be employed for the manufacture. Capsule type may be chosen from commercially available and approved types.

FIG. 11 shows an exemplary 35 mg 3-phenylsulfonyl-8-piperazinyl-1yl-quinoline/10 mg donepezil capsule formulation. 35 mg 3-phenylsulfonyl-8-piperazinyl-1yl-quinoline immediate release tablet/(2) 5 mg donepezil immediate release tablet together in a suitable capsule with or without appropriate backfill excipient. 3-phenylsulfonyl-8-piperazinyl-1yl-quinoline and donepezil tablet may be coated or uncoated, marked or unmarked. Donepezil tablets may be of a standard size produced by an approved generic manufacturer or may be shaped more specifically to fit the capsule. Shape may be round, cylindrical, oval, capsule, or otherwise configured to optimally fit within the volume of the capsule bottom. Tablets will be shaped such that automated capsule filling machinery may be employed for the manufacture. Capsule type may be chosen from commercially available and approved types.

FIG. 12 shows an exemplary 35 mg 3-phenylsulfonyl-8-piperazinyl-1yl-quinoline/10 mg donepezil capsule formulation. 35 mg 3-phenylsulfonyl-8-piperazinyl-1yl-quinoline immediate release tablet/10 mg donepezil immediate release tablet together in a suitable capsule with or without appropriate backfill excipient. 3-phenylsulfonyl-8-piperazinyl-1yl-quinoline and donepezil tablet may be coated or uncoated, marked or unmarked. Donepezil tablets may be of a standard size produced by an approved generic manufacturer or may be shaped more specifically to fit the capsule. Shape may be round, cylindrical, oval, capsule, or otherwise configured to optimally fit within the volume of the capsule bottom. Tablets will be shaped such that automated capsule filling machinery may be employed for the manufacture. Capsule type may be chosen from commercially available and approved types.

FIG. 13 shows an exemplary 35 mg 3-phenylsulfonyl-8-piperazinyl-1yl-quinoline/10 mg donepezil overcoated tablet formulation. 35 mg 3-phenylsulfonyl-8-piperazinyl-1yl-quinoline immediate release tablet/(2) 5 mg donepezil immediate release tablets together in a suitable pharmaceutical or food grade coating. Coating encases three tablets. Coating is of sufficient mechanical strength to resist breakage. Coating is composed of pharmaceutically approved and/or food-grade appropriate constituents. Encasement may be transparent or opaque.

FIG. 14 shows an exemplary 35 mg 3-phenylsulfonyl-8-piperazinyl-1yl-quinoline/10 mg donepezil overcoated tablet formulation. 35 mg 3-phenylsulfonyl-8-piperazinyl-1yl-quinoline immediate release tablet/10 mg donepezil immediate release tablet together in a suitable pharmaceutical or food grade coating. Coating encases three tablets. Coating is of sufficient mechanical strength to resist breakage. Coating is composed of pharmaceutically approved and/or food-grade appropriate constituents. Encasement may be transparent or opaque.

FIG. 15 shows an exemplary 35 mg 3-phenylsulfonyl-8-piperazinyl-1yl-quinoline/5 mg donepezil overcoated tablet formulation. 35 mg 3-phenylsulfonyl-8-piperazinyl-1yl-quinoline immediate release tablet/5 mg donepezil immediate release tablet together in a suitable pharmaceutical or food grade coating. Coating encases three tablets. Coating is of sufficient mechanical strength to resist breakage. Coating is composed of pharmaceutically approved and/or food-grade appropriate constituents. Encasement may be transparent or opaque.

FIG. 16 shows an exemplary 35 mg 3-phenylsulfonyl-8-piperazinyl-1yl-quinoline/5/10 mg donepezil encased product coated edge-to-edge tablet formulation. 35 mg 3-phenylsulfonyl-8-piperazinyl-1 yl-quinoline immediate release tablet/5 mg donepezil immediate release tablet or 35 mg 3-phenylsulfonyl-8-piperazinyl-1yl-quinoline immediate release tablet/10 mg donepezil immediate release tablet together in a suitable pharmaceutical or food grade coating. Coating encases two tablets. Coating is of sufficient mechanical strength to resist breakage. Coating is composed of pharmaceutically approved and/or food-grade appropriate constituents. Encasement may be transparent or opaque.

### DETAILED DESCRIPTION

The present application relates to novel compositions comprising a 5-HT₆ receptor antagonists, specifically 3-phenylsulfonyl-8-piperazinyl-1yl-quinoline or pharmaceutically acceptable salts, hydrates, polymorphs, or solvates thereof and uses thereof, and to the combination of 5-HT₆ receptor antagonists, specifically 3-phenylsulfonyl-8-piperazinyl-1yl-quinoline or pharmaceutically acceptable salts, hydrates, polymorphs, or solvates thereof, with additional therapeutic agents useful for the treatment of a neurodegenerative disease and uses thereof.

In some embodiments, the compounds for use in the methods described herein may be formulated as pharmaceutical compositions. Pharmaceutical compositions of this invention may comprise the compounds described herein or a pharmaceutically acceptable salt thereof and a pharmaceutically acceptable carrier. Such compositions may optionally comprise at least one additional therapeutic agent useful for treating a neurodegenerative disease.

The compounds of this invention may be employed in a conventional manner for controlling the disease described herein, including, but not limited to, a neurodegenerative disease, and for treating diseases or reducing the advancement or severity of effects. Such methods of treatment, their dosage levels and requirements may be selected by those of ordinary skill in the art from available methods and techniques. For example, the compounds of this invention may be combined with a pharmaceutically acceptable adjuvant for administration to a patient suffering from a neurodegenerative disease in a pharmaceutically acceptable manner and in an amount effective to lessen the severity of that disease.

Alternatively, the compounds of this invention may be used in compositions and methods for treating or protecting individuals against the diseases described herein, including but not limited to a neurodegenerative disease, over extended periods of time. The compounds may be employed in such compositions either alone or together with other compounds of this invention in a manner consistent with the conventional utilization of such compounds in pharmaceutical compositions. For example, a compound of this invention may be combined with pharmaceutically acceptable adjuvants conventionally employed in vaccines and administered in prophylactically effective amounts to protect individuals over an extended period of time against the diseases described herein, including, but not limited to, neurodegenerative diseases.

In each of the embodiments disclosed herein, the compounds and methods can be utilized with or on a subject in need of such treatment, which can also be referred to as “in need thereof” As used herein, the phrase “in need thereof” means that the subject has been identified as having a need for the particular method or treatment and that the treatment has been given to the subject for that particular purpose.

The term “patient” and “subject” are interchangeable and may be taken to mean any living organism which may be treated with compounds of the present invention. As such, the terms “patient” and “subject” may include, but is not limited to, any non-human mammal, primate or human. In some embodiments, the “patient” or “subject” is a mammal, such as mice, rats, other rodents, rabbits, dogs, cats, swine, cattle, sheep, horses, primates, or humans. In some embodiments, the patient or subject is an adult, child or infant. In some embodiments, the patient or subject is a human.

As used herein, the terms “combination,” “combined,” and related terms refer to the simultaneous or sequential administration of therapeutic agents in accordance with this invention. For example, a described compound may be administered with another therapeutic agent simultaneously or sequentially in separate unit dosage forms or together in a single unit dosage form. Accordingly, the present invention provides a single unit dosage form comprising a described compound, an additional therapeutic agent, and a pharmaceutically acceptable carrier, adjuvant, or vehicle. Two or more agents are typically considered to be administered “in combination” when a patient or individual is simultaneously exposed to both agents. In many embodiments, two or more agents are considered to be administered “in combination” when a patient or individual simultaneously shows therapeutically relevant levels of the agents in a particular target tissue or sample (e.g., in brain, in serum, etc.).

The term “pharmaceutically acceptable excipient” refers to a non-toxic excipient that may be administered to a patient, together with a compound of this invention, and which does not destroy the pharmacological activity thereof. Pharmaceutically acceptable excipients that may be used in these compositions include, but are not limited to, ion exchangers, alumina, aluminum stearate, lecithin, serum proteins such as human serum albumin, buffer substances such as phosphates, glycine, sorbic acid, potassium sorbate, partial glyceride mixtures of saturated vegetable fatty acids, water, salts or electrolytes such as protamine sulfate, disodium hydrogen phosphate, potassium hydrogen phosphate, sodium chloride, zinc salts, colloidal silica, magnesium tri silicate, polyvinyl pyrrolidone, cellulose-based substances, polyethylene glycol, sodium carboxymethylcellulose, polyacrylates, waxes, polyethylene-polyoxypropylene-block polymers, polyethylene glycol and wool fat. Pharmaceutically acceptable excipients that may be used in the pharmaceutical compositions of this invention include, but are not limited to, ion exchangers, alumina, aluminum stearate, lecithin, serum proteins, such as human serum albumin, buffer substances such as phosphates, glycine, sorbic acid, potassium sorbate, partial glyceride mixtures of saturated vegetable fatty acids, water, salts or electrolytes, such as protamine sulfate, disodium hydrogen phosphate, potassium hydrogen phosphate, sodium chloride, zinc salts, colloidal silica, magnesium tri silicate, polyvinyl pyrrolidone, cellulose-based substances, polyethylene glycol, sodium carboxymethylcellulose, polyacrylates, waxes, polyethylene-polyoxypropylene-block polymers, wool fat and self-emulsifying drug delivery systems (SEDDS) such as α-tocopherol, polyethyleneglycol 1000 succinate, or other similar polymeric delivery matrices.

The term “therapeutically effective amount” as used herein refers to the amount of active compound or pharmaceutical agent that elicits the biological or medicinal response in a tissue, system, animal, individual or human that is being sought by a researcher, veterinarian, medical doctor or other clinician, which includes one or more of the following: (1) Preventing the disease; for example, preventing a disease, condition or disorder in an individual that may be predisposed to the disease, condition or disorder but does not yet experience or display the pathology or symptomatology of the disease, (2) Inhibiting the disease; for example, inhibiting a disease, condition or disorder in an individual that is experiencing or displaying the pathology or symptomatology of the disease, condition or disorder (i.e., arresting further development of the pathology and/or symptomatology), and (3) Ameliorating the disease; for example, ameliorating a disease, condition or disorder in an individual that is experiencing or displaying the pathology or symptomatology of the disease, condition or disorder (i.e., reversing the pathology and/or symptomatology). In some embodiments, the therapeutically effective amount of a compound represents the daily dose a particular compound. In some embodiments, the daily dose of a particular compound may be administered as a single daily dose or may be divided into two or more doses of equal or unequal amounts administered throughout the day.

The term “sub therapeutic amount” as used herein refers to a dosage that is below that typically used for the subject agent in typical therapeutic or prophylactic use.

As used herein, “fixed-dose-combination or FDC” refers to a combination of two drugs or active ingredients presented in a single dosage unit such as a tablet or oral dosage form. When formulating a solid fixed dose combination, the objective is to provide a patient-convenient combination dosage form of active ingredients that is bioequivalent to the corresponding free-combination of the same active ingredients.

Alkyl groups, whether alone or as part of another group, may be straight chain or branched and the groups alkoxy and alkanoyl shall be interpreted similarly. Alkyl moieties are more preferably C₁₋₄ alkyl, eg. methyl or ethyl. The term ‘halogen’ is used herein to describe, unless otherwise stated, a group selected from fluorine, chlorine, bromine or iodine.

The term “aryl” includes phenyl and naphthyl. The term “heteroaryl” is intended to mean a 5-7 membered monocyclic aromatic or a fused 8-10 membered bicyclic aromatic ring containing 1 to 3 heteroatoms selected from oxygen, nitrogen and sulphur. Suitable examples of such monocyclic aromatic rings include thienyl, furyl, pyrrolyl, triazolyl, imidazolyl, oxazolyl, thiazolyl, oxadiazolyl, isothiazolyl, isoxazolyl, thiadiazolyl, pyrazolyl, pyrimidyl, pyridazinyl, pyrazinyl and pyridyl. Suitable examples of such fused aromatic rings include benzofused aromatic rings such as quinolinyl, isoquinolinyl, quinazolinyl, quinoxalinyl, cinnolinyl, naphthyridinyl, indolyl, indazolyl, pyrrolopyridinyl, benzofuranyl, benzothienyl, benzimidazolyl, benzoxazolyl, benzisoxazolyl, benzothiazolyl, benzisothiazolyl, benzoxadiazolyl, benzothiadiazolyl and the like. Heteroaryl groups, as described above, may be linked to the remainder of the molecule via a carbon atom or, when present, a suitable nitrogen atom except where otherwise indicated above. It will be appreciated that wherein the above mentioned aryl or heteroaryl groups have more than one substituent, said substituents may be linked to form a ring, for example a carboxyl and amine group may be linked to form an amide group.

The compounds described herein can form acid addition salts thereof. It will be appreciated that for use in medicine the salts of the compounds described herein should be pharmaceutically acceptable. Suitable pharmaceutically acceptable salts will be apparent to those skilled in the art and include those described in J. Pharm. Sci., 1977, 66, 1-19, such as acid addition salts formed with inorganic acids e.g. hydrochloric, hydrobromic, sulfuric, nitric or phosphoric acid; and organic acids e.g. succinic, maleic, acetic, fumaric, citric, tartaric, benzoic, p-toluenesulfonic, methanesulfonic or naphthalenesulfonic acid. The present invention includes within its scope all possible stoichiometric and non-stoichiometric forms.

The compounds described herein may be prepared in crystalline or non-crystalline form, and, if crystalline, may optionally be solvated, eg. as the hydrate. This invention includes within its scope stoichiometric solvates (eg. hydrates) as well as compounds containing variable amounts of solvent (eg. water). Certain compounds described herein are capable of existing in stereoisomeric forms (e.g. diastereomers and enantiomers) and the invention extends to each of these stereoisomeric forms and to mixtures thereof including racemates. The different stereoisomeric forms may be separated one from the other by the usual methods, or any given isomer may be obtained by stereospecific or asymmetric synthesis. The invention also extends to any tautomeric forms and mixtures thereof.

Although any methods and materials similar or equivalent to those described herein can be used in the practice or testing of embodiments of the present invention, the preferred methods, devices, and materials are now described.

In some embodiments, the 5-HT₆ receptor antagonist is a compound of formula (I):

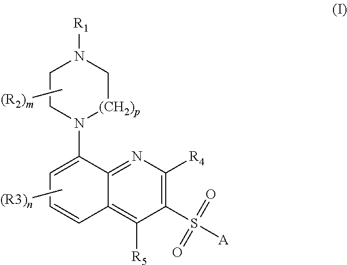

wherein:

R₁ and R₂ independently represent hydrogen or C₁₋₆ alkyl or R₁ is linked to R₂ to form a group (CH₂)₂, (CH₂)₃ or (CH₂)₄; R₃, R₄ and R₅ independently represent hydrogen, halogen, cyano, —CF₃, —CF₃O, C₁₋₆ alkyl, C₁₋₆ alkoxy, C₁₋₆ alkanoyl or a group CONR₆R₇; R₆ and R⁷ independently represent hydrogen or C₁₋₆ alkyl or together may be fused to form a 5- to 7-membered aromatic or non-aromatic heterocyclic ring optionally interrupted by an O or S atom; m represents an integer from 1 to 4, such that when m is an integer greater than 1, two R₂ groups may instead be linked to form a group CH₂, (CH₂)₂ or (CH₂)₃; n represents an integer from 1 to 3; p represents 1 or 2; A represents a group —Ar¹ or —Ar²Ar³; Ar¹, Ar² and Ar³ independently represent an aryl group or a heteroaryl group, both of which may be optionally substituted by one or more (e.g., 1, 2 or 3) substituents which may be the same or different, and which are selected from the group consisting of halogen, hydroxy, cyano, nitro, trifluoromethyl, trifluoromethoxy, C₁₋₆ alkyl, trifluoromethanesulfonyloxy, pentafluoroethyl, C₁₋₆ alkoxy, arylC₁₋₆ alkoxy, C₁₋₆ alkylthio, C₁₋₆ alkoxyC₁₋₆ alkyl, C₃₋₇cycloalkylC₁₋₆ alkoxy, C₁₋₆ alkanoyl, C₁₋₆ alkoxycarbonyl, C₁₋₆ alkyl sulfonyl, C₁₋₆ alkyl sulfinyl, C₁₋₆ alkyl sulfonyl oxy, C₁₋₆alkyl sulfonyl C₁₋₆ alkyl, arylsulfonyl, aryl sulfonyloxy, aryl sulfonyl C₁₋₆ alkyl, C₁₋₆ alkylsulfonamido, C₁₋₆ alkylamido, C₁₋₆ alkylsulfonamido C₁₋₆ alkyl, C₁₋₆ alkylamidoC₁₋₆ alkyl, arylsulfonamido, arylcarboxamido, arylsulfonamido C₁₋₆ alkyl, arylcarboxamido C₁₋₆ alkyl, aroyl, aroylC₁₋₆ alkyl, arylC₁₋₆ alkanoyl, or a group CONR₈R₉ or SO₂NR₈R₉, wherein R₈ and R₉ independently represent hydrogen or C₁₋₆ alkyl or together may be fused to form a 5- to 7-membered aromatic or non-aromatic heterocyclic ring optionally interrupted by an O or S atom; or pharmaceutically acceptable salts, hydrates or solvates thereof.

Alkyl groups, whether alone or as part of another group, may be straight chain or branched and the group's alkoxy and alkanoyl shall be interpreted similarly. Alkyl moieties are more preferably C₁₋₄ alkyl, e.g., methyl or ethyl. The term ‘halogen’ is used herein to describe, unless otherwise stated, a group selected from fluorine, chlorine, bromine or iodine.

The term “aryl” includes phenyl and naphthyl. The term “heteroaryl” is intended to mean a 5-7 membered monocyclic aromatic or a fused 8-10 membered bicyclic aromatic ring containing 1 to 3 heteroatoms selected from oxygen, nitrogen and sulphur. Suitable examples of such monocyclic aromatic rings include thienyl, furyl, pyrrolyl, triazolyl, imidazolyl, oxazolyl, thiazolyl, oxadiazolyl, isothiazolyl, isoxazolyl, thiadiazolyl, pyrazolyl, pyrimidyl, pyridazinyl, pyrazinyl and pyridyl. Suitable examples of such fused aromatic rings include benzofused aromatic rings such as quinolinyl, isoquinolinyl, quinazolinyl, quinoxalinyl, cinnolinyl, naphthyridinyl, indolyl, indazolyl, pyrrolopyridinyl, benzofuranyl, benzothienyl, benzimidazolyl, benzoxazolyl, benzisoxazolyl, benzothiazolyl, benzisothiazolyl, benzoxadiazolyl, benzothiadiazolyl and the like. Heteroaryl groups, as described above, may be linked to the remainder of the molecule via a carbon atom or, when present, a suitable nitrogen atom except where otherwise indicated above. It will be appreciated that wherein the above mentioned aryl or heteroaryl groups have more than one substituent, said substituents may be linked to form a ring, for example a carboxyl and amine group may be linked to form an amide group.

In some embodiments, R₁ represents hydrogen, methyl, ethyl, isopropyl, isobutyl or 2,2-dimethylpropyl. In some embodiments, R₁ represents hydrogen or methyl, especially hydrogen. Preferably R₂ represents hydrogen, methyl (e.g., 3-methyl, 2-methyl, 3,3-dimethyl or 2,5-dimethyl) or is linked to R₁ to form a (CH₂)₃ group. More preferably, R₂ represents hydrogen or methyl (e.g., 3-methyl), especially hydrogen.

In some embodiments, R₃ represents hydrogen, methyl (e.g., 6-methyl) or halogen (e.g., 7-chloro). More preferably, R₃ represents hydrogen.

In some embodiments, R₄ and R₅ independently represent hydrogen or methyl, especially hydrogen.

In some embodiments, n represents 1. In some embodiments, m and p independently represent 1 or 2, more preferably m and p both represent 1. In some embodiments, m represents 2 and both R₂ groups are linked to form a CH₂ group linking C-2 and C-5 of the piperazine ring.

In some embodiments, when A represents a group —Ar¹, Ar¹ preferably represents optionally substituted phenyl or pyridyl, or in some embodiments, a phenyl optionally substituted with halogen (e.g., chlorine, fluorine or bromine), cyano, trifluoromethyl or trifluoromethoxy. In some embodiments, Ar¹ is unsubstituted phenyl or phenyl substituted by halogen (e.g., 2-chloro, 3-chloro, 4-chloro, 2-fluoro, 3-fluoro, 4-fluoro or 4-bromo), C₁₋₆ alkyl (e.g., 2-methyl or 4-methyl), C₁₋₆ alkoxy (e.g., 2-methoxy), trifluoromethyl (e.g., 2-trifluoromethyl or 3-trifluoromethyl) or trifluoromethoxy (e.g., 2-trifluoromethoxy).

In some embodiments, when A represents a group —Ar²—Ar³, Ar^{e} and Ar^{a} both independently represent phenyl or monocyclic heteroaryl group as defined above. In some embodiments, A represents a group —Ar¹. In some embodiments, —Ar¹ is unsubstituted phenyl.

The compounds of formula (I) can form acid addition salts thereof. It will be appreciated that for use in medicine the salts of the compounds of formula (I) should be pharmaceutically acceptable. Suitable pharmaceutically acceptable salts will be apparent to those skilled in the art and include those described in J. Pharm. Sci., 1977, 66, 1-19, such as acid addition salts formed with inorganic acids, e.g., hydrochloric, hydrobromic, sulfuric, nitric or phosphoric acid; and organic acids, e.g., succinic, maleic, acetic, fumaric, citric, tartaric, benzoic, p-toluenesulfonic, methanesulfonic or naphthalenesulfonic acid. The present invention includes within its scope all possible stoichiometric and non-stoichiometric forms.

The compounds of formula (I) may be prepared in crystalline or non-crystalline form, and, if crystalline, may optionally be solvated, e.g., as the hydrate. This invention includes within its scope stoichiometric solvates (e.g., hydrates) as well as compounds containing variable amounts of solvent (e.g., water). Certain compounds of formula (I) are capable of existing in stereoisomeric forms (e.g., diastereomers and enantiomers) and the invention extends to each of these stereoisomeric forms and to mixtures thereof including racemates. The different stereoisomeric forms may be separated one from the other by the usual methods, or any given isomer may be obtained by stereospecific or asymmetric synthesis. The invention also extends to any tautomeric forms and mixtures thereof.

Embodiments herein are directed to compositions comprising: a therapeutically effective amount of 3-phenylsulfonyl-8-piperazinyl-1yl-quinoline (Formula II)

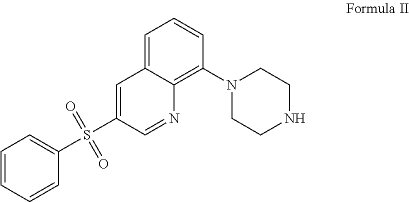 [SMILES: O=S(=O)(c1ccccc1)c1cnc2c(N3CCNCC3)cccc2c1]

or pharmaceutically acceptable salts, hydrates or solvates thereof; a therapeutically effective amount of at least one additional therapeutic agent useful for the treatment of a neurodegenerative disease; and at least one pharmaceutically acceptable excipient; wherein the composition is suitable for oral administration.

In some embodiments, the neurodegenerative disease is selected from Alzheimer's disease (including mild or early-stage Alzheimer's disease, mild to moderate Alzheimer's disease, moderate or mid-stage Alzheimer's disease, moderate to severe Alzheimer's disease, moderately severe Alzheimer's disease, severe Alzheimer's disease, Alzheimer's disease with Lewy bodies, (AD)), Parkinson's disease (including Parkinson's disease chemically induced by exposure to environmental agents such as pesticides, insecticides, or herbicides and/or metals such as manganese, aluminum, cadmium, copper, or zinc, SNCA gene-linked Parkinson's disease, sporadic or idiopathic Parkinson's disease, or Parkin- or LRRK2-linked Parkinson's disease (PD)), autosomal-dominant Parkinson's disease, Diffuse Lewy Body Disease (DLBD) also known as Dementia with Lewy Bodies (DLB), Pure Autonomic Failure, Lewy body dysphagia, Incidental LBD, Inherited LBD (e.g., mutations of the alpha-synuclein gene, PARK3 and PARK4), multiple system atrophy (including Olivopontocerebellar Atrophy, Striatonigral Degeneration, Shy-Drager Syndrome (MSA)), combined Alzheimer's and Parkinson disease and/or MSA, Huntington's disease, synucleinopathies, disorders or conditions characterized by the presence of Lewy bodies, multiple sclerosis, Amyotrophic lateral sclerosis (ALS) dementia (including vascular dementia, Lewy body dementia, Parkinson's dementia, frontotemporal dementia), Down syndrome, Psychosis (including agitation caused by a neurodegenerative disease or associated with dopaminergic therapy such as but not limited to Parkinson's disease psychosis, Alzheimer's disease psychosis, Lewy body dementia psychosis), dyskinesia (including agitation caused by a neurodegenerative disease or associated with dopaminergic therapy), agitation (including agitation caused by a neurodegenerative disease or associated with dopaminergic therapy), conditions associated with dopaminergic therapy (including dystonia, myoclonus, or tremor), synucleinopathies, diseases, disorders or conditions associated with abnormal expression, stability, activities and/or cellular processing of α-synuclein, diseases, disorders or conditions characterized by the presence of Lewy bodies, and combinations thereof.

In some embodiments, the therapeutically effective amount of 3-phenylsulfonyl-8-piperazinyl-1 yl-quinoline or pharmaceutically acceptable salts, hydrates or solvates thereof, and a therapeutically effective amount of at least one additional therapeutic agent useful for treating a neurodegenerative disease, are configured as a single subunit, or two or more subunits.

In some embodiments, the at least one pharmaceutical acceptable excipient is configured into the single subunit, or the two or more subunits.

In some embodiments, the single subunit comprises a bar, beads, a block, particles, pellets, granules, fibers, globules, powders, a pill, a capsule, a tablet, a caplet, an orally disintegrating tablet, an osmotic controlled-release oral delivery system and any combination thereof.

In some embodiments, the tablet is a monolayer tablet, a bilayer tablet, or a multilayer tablet or a combination thereof.

In some embodiments, the single subunit further comprises an encapsulation medium.

In some embodiments, the encapsulation medium is a capsule, a soft gel cap, a gel cap, a coating, or any combination thereof.

In some embodiments, the coating comprises a membrane, a film, a wax, a varnish, a glaze, a polymer coating, a sugar coating, a polysaccharide based coating, an enteric coating, or a combination thereof.

In some embodiments, the therapeutically effective amount of 3-phenylsulfonyl-8-piperazinyl-1yl-quinoline or pharmaceutically acceptable salts, hydrates or solvates thereof, and a therapeutically effective amount of at least one additional therapeutic agent useful for treating a neurodegenerative disease are independently configured for immediate release, sustained release, extended release, or any combination thereof.

In some embodiments, the therapeutically effective amount of 3-phenylsulfonyl-8-piperazinyl-1yl-quinoline or pharmaceutically acceptable salts, hydrates or solvates thereof is configured for immediate release, and the additional therapeutic agent useful for treating a neurodegenerative disease is configured for immediate release, sustained release, extended release, or any combination thereof.

In some embodiments, the therapeutically effective amount of 3-phenylsulfonyl-8-piperazinyl-1yl-quinoline or pharmaceutically acceptable salts, hydrates or solvates thereof is configured for sustained release, and the additional therapeutic agent useful for treating a neurodegenerative disease is configured for immediate release, sustained release, extended release, or any combination thereof.

In some embodiments, the therapeutically effective amount of 3-phenylsulfonyl-8-piperazinyl-1yl-quinoline or pharmaceutically acceptable salts, hydrates or solvates thereof is configured for extended release, and the additional therapeutic agent useful for treating a neurodegenerative disease is configured for immediate release, sustained release, extended release, or any combination thereof.

In some embodiments, the two or more subunits independently comprise a bar, beads, a block, particles, pellets, granules, fibers, globules, powders, a pill, a capsule, a tablet, a caplet, an orally disintegrating tablet, an osmotic controlled-release oral delivery system and any combination thereof.

In some embodiments, the tablet is a monolayer tablet, a bilayer tablet, or a multilayer tablet or a combination thereof.

In some embodiments, the two or more subunits comprise a therapeutically effective amount of 3-phenylsulfonyl-8-piperazinyl-1yl-quinoline or pharmaceutically acceptable salts, hydrates or solvates thereof is configured into a first subunit, and the therapeutically effective amount of at least one additional therapeutic agent useful for the treatment of neurodegenerative disease configured into at least one additional subunit.

In some embodiments, the first subunit and the at least one additional subunits are combined into an encapsulation medium.

In some embodiments, wherein the encapsulation medium is a capsule, a soft gel cap, a gel cap, a coating, or any combination thereof.

In some embodiments, the coating comprises a membrane, a film, a wax, a varnish, a glaze, a polymer coating, a sugar coating, a polysaccharide based coating, an enteric coating, or a combination thereof.

In some embodiments, the two or more subunits independently comprise a bar, beads, a block, particles, pellets, granules, fibers, globules, powders, a pill, a capsule, a tablet, a caplet, an orally disintegrating tablet, an osmotic controlled-release oral delivery system and any combination thereof.

In some embodiments, the tablet is a monolayer tablet, a bilayer tablet, or a multilayer tablet or a combination thereof.

In some embodiments, the two or more subunits comprise the therapeutically effective amount of 3-phenylsulfonyl-8-piperazinyl-1yl-quinoline or pharmaceutically acceptable salts, hydrates or solvates thereof configured into a first subunit, and the therapeutically effective amount of at least one additional therapeutic agent useful for the treatment of neurodegenerative disease configured into at least one additional subunit.

In some embodiments, the first subunit and the at least one additional subunits are combined into a encapsulation medium.

In some embodiments, wherein the encapsulation medium is a capsule, a soft gel cap, a gel cap, a coating, or any combination thereof.

In some embodiments, the coating comprises a membrane, a film, a wax, a varnish, a glaze, a polymer coating, a sugar coating, a polysaccharide based coating, an enteric coating, or a combination thereof.

In some embodiments, the therapeutically effective amount of 3-phenylsulfonyl-8-piperazinyl-1yl-quinoline or pharmaceutically acceptable salts, hydrates, polymorphs, or solvates thereof is from about 0.001 mg to about 1,000 mg, about 0.001 mg to about 200 mg, about 0.001 mg to about 175 mg, or 0.001 mg to about 70 mg

In some embodiments, the therapeutically effective amount of 3-phenylsulfonyl-8-piperazinyl-1yl-quinoline or pharmaceutically acceptable salts, hydrates, polymorphs, or solvates thereof is about 15 mg, about 35 mg, or about 70 mg.

In some embodiments, the therapeutically effective amount of 3-phenylsulfonyl-8-piperazinyl-1yl-quinoline or pharmaceutically acceptable salts, hydrates, polymorphs, or solvates thereof is an amount selected from the group consisting of an amount of 3-phenylsulfonyl-8-piperazinyl-1yl-quinoline that may cause convulsions in a subject to which it is administered; an amount that would be expected to exceed the maximum tolerated dose for the subject to which it is administered; an amount associated with systemic exposures characterized by an AUCtau-ss of about 8.2 μg·h/ml, a Cmax of about 0.26 μg/ml; or a combination thereof an mount associated with systemic exposures characterized by an AUC, Cmax, or combinations thereof, that are about 2 to about 3 times higher than the mean clinical exposure achieved at the proposed clinical dose for monotherapy with 3-phenylsulfonyl-8-piperazinyl-1yl-quinoline (i.e. mean AUCtau-ss of about 3.2 μg·h/ml and Cmax of about 0.180 μg/ml), an amount associated with a recorded systemic clinical exposure that is greater than the highest recorded systemic clinical exposure (AUC0-∞ of about 9.25 μg·h/ml and Cmax of about 0.293 μg/ml), an amount of 3-phenylsulfonyl-8-piperazinyl-1yl-quinoline that is greater than about 10 mg/kg/day, an amount of 3-phenylsulfonyl-8-piperazinyl-1yl-quinoline that is greater than 15 mg/day, a dose of 3-phenylsulfonyl-8-piperazinyl-1yl-quinoline that is greater than about 35 mg/day or any combination thereof.

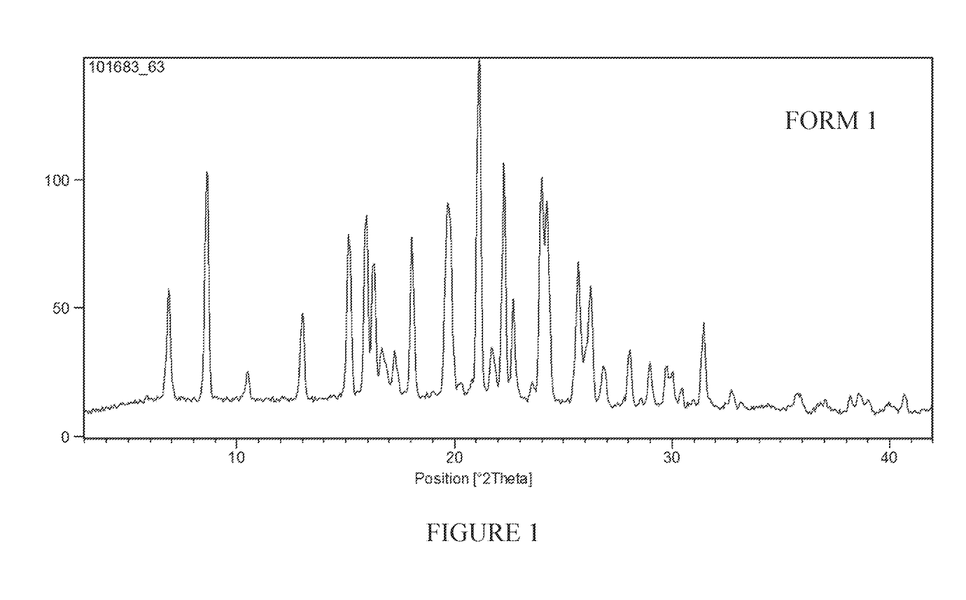

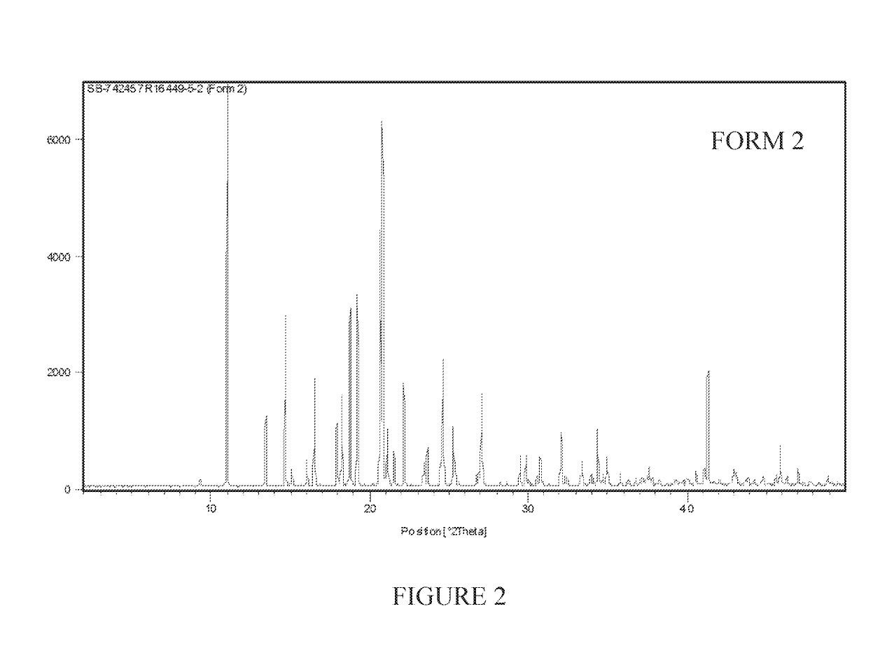

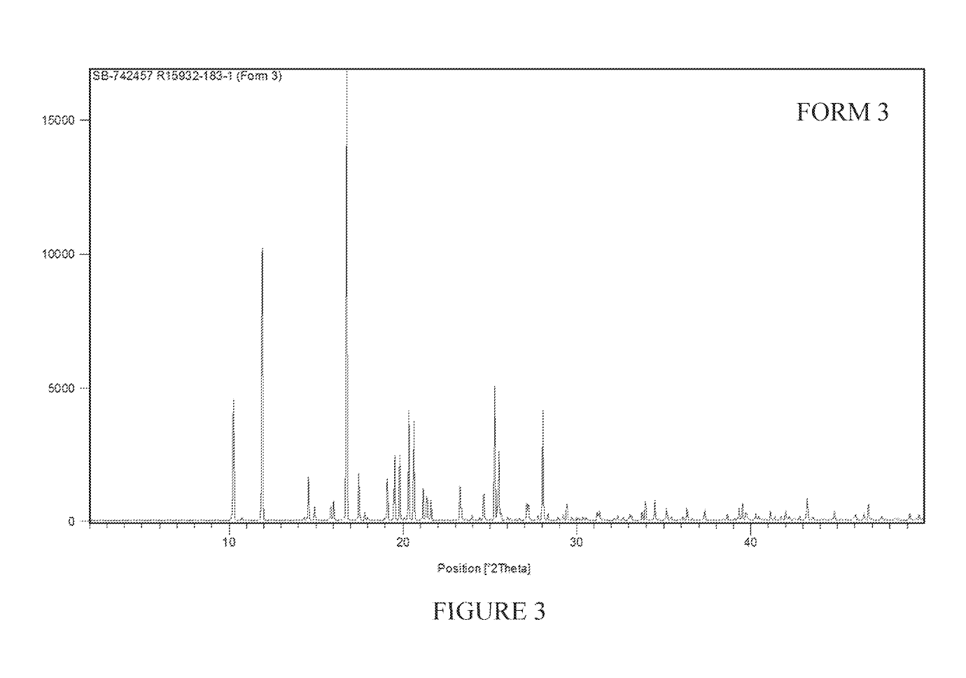

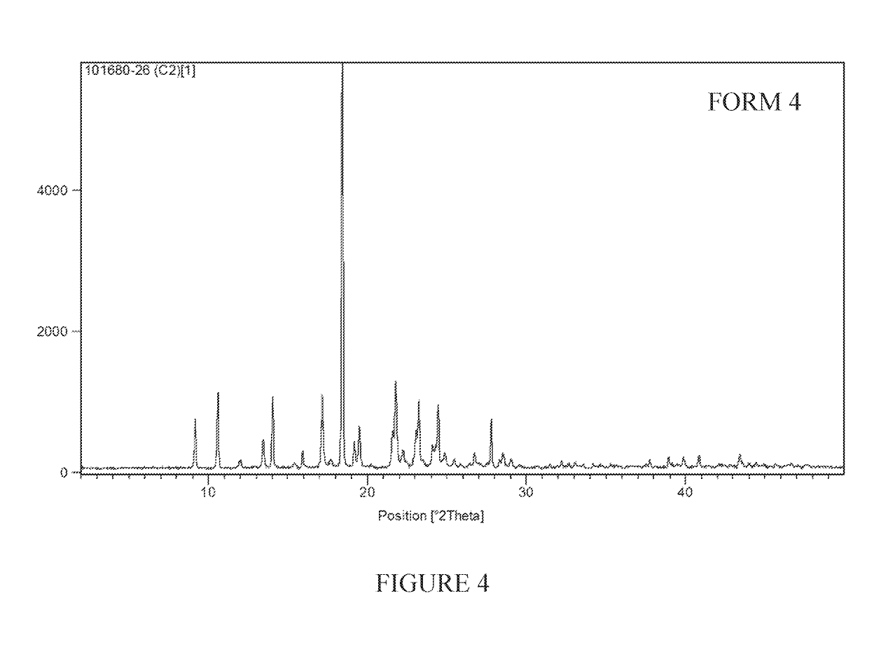

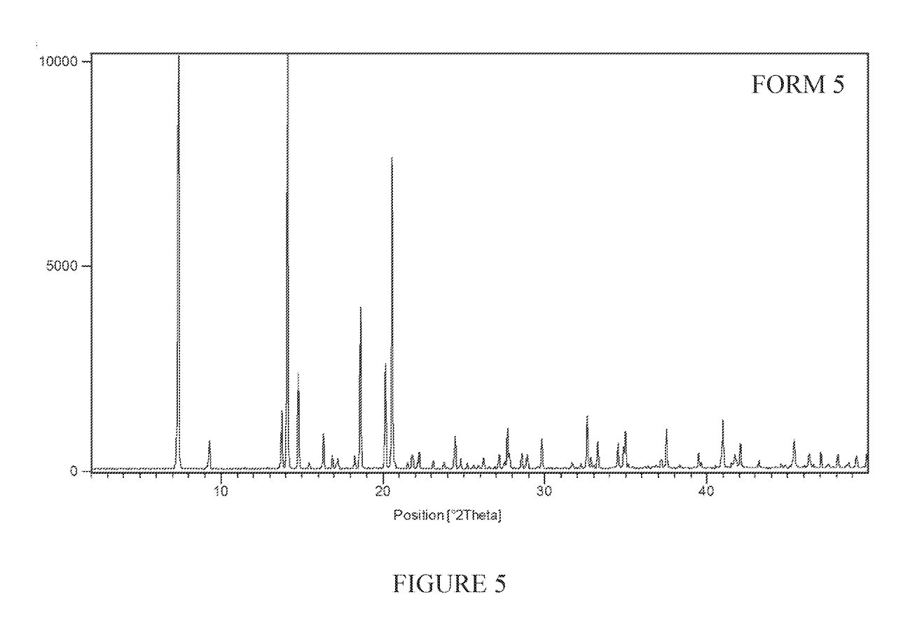

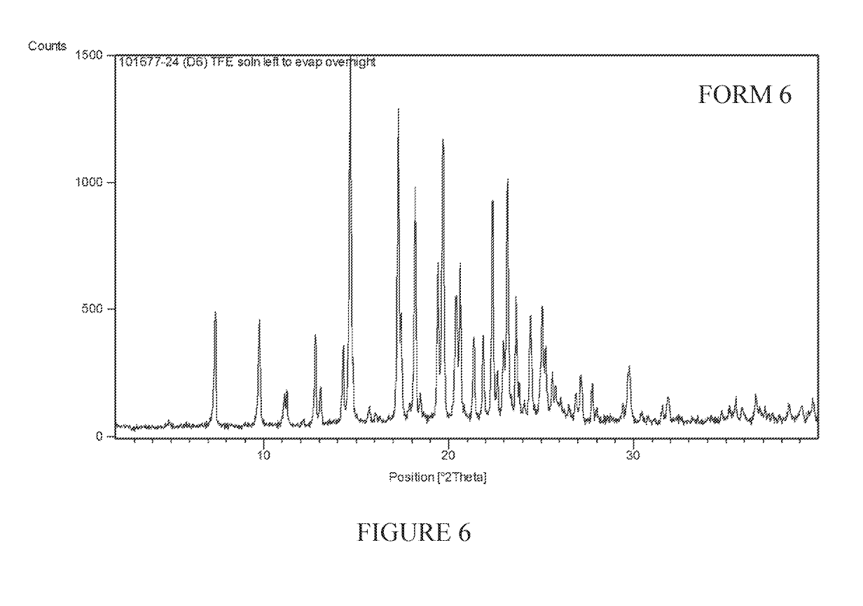

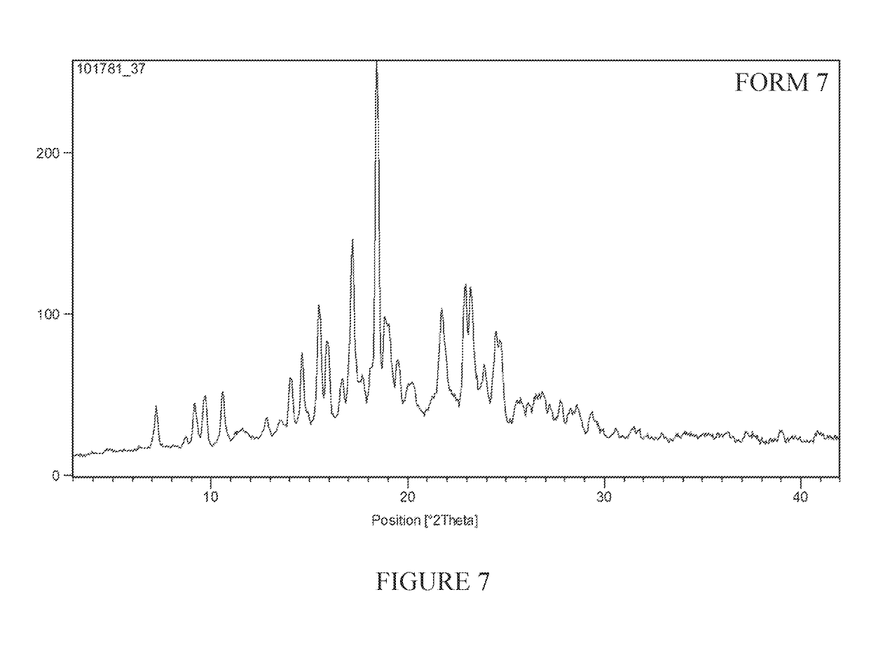

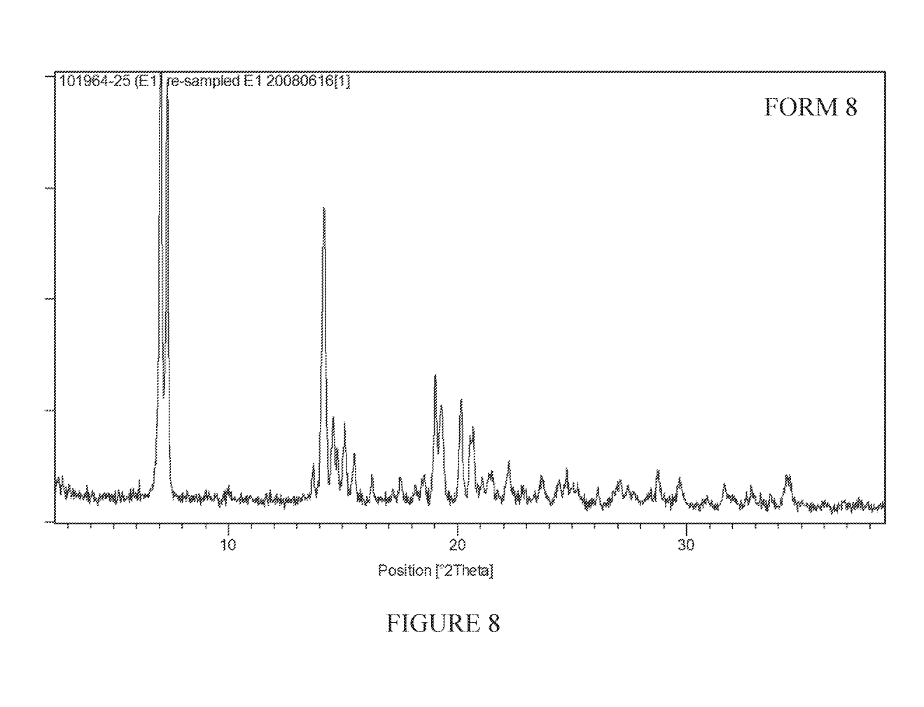

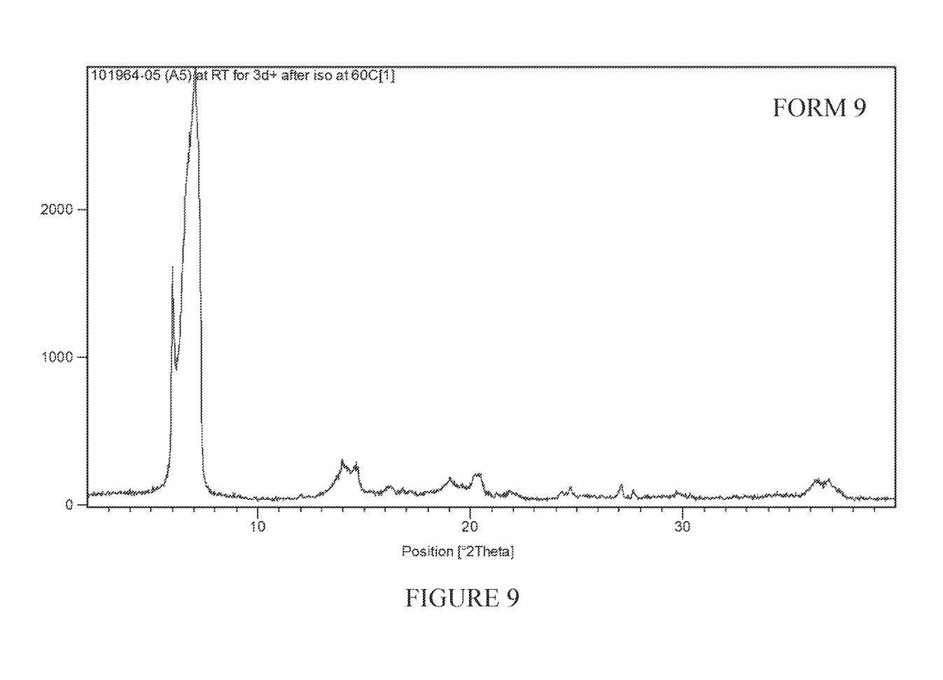

Some embodiments are directed to pharmaceutical compositions comprising a therapeutically effective amount of 3-phenylsulfonyl-8-piperazinyl-1yl-quinoline or pharmaceutically acceptable salts, hydrates, polymorphs, or solvates thereof and at least one pharmaceutically acceptable excipient; wherein the therapeutically effective amount of 3-phenylsulfonyl-8-piperazinyl-1yl-quinoline comprises at least one polymorphic form of 3-phenylsulfonyl-8-piperazinyl-1 yl-quinoline. In some embodiments, the at least one polymorphic form is characterized by a powder x-ray diffraction substantially as shown in any one of FIGS. 1-9. In some embodiments, the at least one polymorphic form is characterized by a powder x-ray diffraction substantially as shown in FIG. 2. In some embodiments, the at least one polymorphic form is characterized by a powder x-ray diffraction substantially as shown in FIG. 3. In some embodiments, the at least one polymorphic form is characterized by a powder x-ray diffraction substantially as shown in FIG. 4. In some embodiments, the at least one polymorphic form is characterized by a powder x-ray diffraction substantially as shown in FIG. 5. In some embodiments, the at least one polymorphic form is characterized by a powder x-ray diffraction substantially as shown in FIG. 6. In some embodiments, the at least one polymorphic form is characterized by a powder x-ray diffraction substantially as shown in FIG. 7. In some embodiments, the at least one polymorphic form is characterized by a powder x-ray diffraction substantially as shown in FIG. 8. In some embodiments, the at least one polymorphic form is characterized by a powder x-ray diffraction substantially as shown in FIG. 9.

In some embodiments, the at least one additional therapeutic agent is selected from the group consisting of an acetylcholinesterase inhibitor, an NMDA receptor antagonist, a 5HT_{2A} inverse agonist or any combination thereof.

In some embodiments, the acetylcholinesterase inhibitor is selected from the group consisting of donepezil, rivastigmine, galantamine, physostigmine, neostigmine, pyridostigmine, ambenonium, demecarium, a phenanthrene derivative, galantamine, caffeine, a piperidine tacrine (also known as tetrahydroaminoacridine), edrophonium, huperzine A, ladostigil, ungeremine, lactucopicrin, 6-[(3-cyclobutyl-2,3,4,5-tetrahydro-1H-3-benzazepin-7-yl)oxy]-N-methyl-3-pyridinecarboxamide hydrochloride or 1-{6-[(3-cyclobutyl-2,3,4,5-tetrahydro-1H-3-benzazepin-7-yl)oxy]-3-pyridinyl}-2-pyrrolidinone or pharmaceutically acceptable salts, hydrates, polymorphs, or solvates thereof.

In some embodiments, the acetylcholinesterase inhibitor is donepezil. In some embodiments, donepezil or pharmaceutically acceptable salts, hydrates, polymorphs, or solvates thereof is donepezil hydrochloride.

In some embodiments, the therapeutically effective amount of donepezil or pharmaceutically acceptable salts, hydrates, polymorphs, or solvates thereof is configured for immediate release, extended release, delayed release, or any combination thereof.

In some embodiments, the therapeutically effective amount of donepezil or pharmaceutically acceptable salts, hydrates, polymorphs, or solvates thereof is from about 0.001 mg to about 1,000 mg, or about 0.001 mg to about 30 mg.

In some embodiments, the therapeutically effective amount of donepezil or pharmaceutically acceptable salts, hydrates, polymorphs, or solvates thereof is about 5 mg, 10 mg, or 23 mg.

In some embodiments, the therapeutically effective amount in an acetylcholinesterase inhibitor is administered to a subject in need thereof in a sub therapeutic amount. In some embodiments, donepezil or pharmaceutically acceptable salts, hydrates or solvates thereof is administered to a subject in need thereof in a daily dose that is considered to sub therapeutic.

In some embodiments, the acetylcholinesterase inhibitor is rivastigmine. In some embodiments, the rivastigmine is rivastigmine tartrate. In some embodiments, the therapeutically effective amount of rivastigmine or pharmaceutically acceptable salts, hydrates, polymorphs, or solvates thereof is configured for immediate release, for extended release, delayed release, or any combination thereof. In some embodiments, the therapeutically effective amount of rivastigmine or pharmaceutically acceptable salts, hydrates, polymorphs, or solvates thereof is from about 0.001 mg to about 1,000 mg, or about 0.001 mg to about 15 mg. In some embodiments, the therapeutically effective amount of rivastigmine or pharmaceutically acceptable salts, hydrates, polymorphs, or solvates thereof is about 1.5 mg, about 3 mg, about 4.5 mg, about 6 mg, about 9 mg, about 9.5 mg, about 12 mg, or about 13.3 mg. In some embodiments, rivastigmine or pharmaceutically acceptable salts, hydrates or solvates thereof is administered to a subject in need thereof in a daily dose that is considered to sub therapeutic.

In some embodiments, the acetylcholinesterase inhibitor is galantamine. In some embodiments, galantamine or pharmaceutically acceptable salts, hydrates, polymorphs, or solvates thereof is galantamine hydrobromide. In some embodiments, the therapeutically effective amount of galantamine or pharmaceutically acceptable salts, hydrates, polymorphs, or solvates thereof is configured for immediate release, extended release, delayed release, or any combination thereof. In some embodiments, the therapeutically effective amount of galantamine or pharmaceutically acceptable salts, hydrates, polymorphs, or solvates thereof is from about 0.001 mg to about 1,000 mg, or about 0.001 mg to about 30 mg. In some embodiments, the therapeutically effective amount of galantamine or pharmaceutically acceptable salts, hydrates, polymorphs, or solvates thereof is about 4 mg, about 8 mg, about 12 mg, about 16 mg, or about 24 mg. In some embodiments, galantamine or pharmaceutically acceptable salts, hydrates or solvates thereof is administered to a subject in need thereof in an amount that is considered to be sub therapeutic.

In some embodiments, NMDA receptor antagonist is selected from the group consisting of memantine, amantadine, and ketamine. In some embodiments, the NMDA receptor antagonist is memantine. In some embodiments, the memantine or pharmaceutically acceptable salts, hydrates, polymorphs, or solvates thereof comprises memantine hydrochloride. In some embodiments, the therapeutically effective amount of memantine or pharmaceutically acceptable salts, hydrates, polymorphs, or solvates thereof is configured for extended release, delayed release or any combination thereof. In some embodiments, the therapeutically effective amount of memantine or pharmaceutically acceptable salts, hydrates, polymorphs, or solvates thereof is from about 0.001 mg to about 1,000 mg, or about 0.001 mg to about 30 mg. In some embodiments, the therapeutically effective amount of memantine or pharmaceutically acceptable salts, hydrates, polymorphs, or solvates thereof is about 5 mg, about 7 mg, about 10 mg, about 14 mg, about 20 mg, about 21 mg, or about 28 mg. In some embodiments, memantine or pharmaceutically acceptable salts, hydrates or solvates thereof is administered to a subject in need thereof in an amount that is considered to be sub therapeutic.

In some embodiments, the NMDA receptor antagonist is amantadine. In some embodiments, the amantadine or pharmaceutically acceptable salts, hydrates, polymorphs, or solvates thereof comprises amantadine hydrochloride.

In some embodiments, the therapeutically effective amount of amantadine or pharmaceutically acceptable salts, hydrates, polymorphs, or solvates thereof is configured for immediate release, extended release, delayed release, or any combination thereof.

In some embodiments, the therapeutically effective amount of amantadine or pharmaceutically acceptable salts, hydrates, polymorphs, or solvates thereof is from about 0.001 mg to about 1,000 mg, or about 0.001 mg to about 500 mg. In some embodiments, amantadine or pharmaceutically acceptable salts, hydrates or solvates thereof is administered to a subject in need thereof in an amount that is considered to sub therapeutic.

In some embodiments, the therapeutically effective amount of amantadine or pharmaceutically acceptable salts, hydrates, polymorphs, or solvates thereof is from about 100 mg to about 400 mg.

In some embodiments, the therapeutically effective amount of amantadine or pharmaceutically acceptable salts, hydrates, polymorphs, or solvates thereof is about 100 mg, 200 mg, 300 mg or about 400 mg.

In some embodiments, the 5-HT2A inverse agonist is nelotanserin, pimavanserin, pruvanserin, eplivanserin, volinanserin, glemanserin, ketanserin, ritanserin, clozapine, or pharmaceutically acceptable salts, hydrates, polymorphs, or solvates thereof.

In some embodiments, the nelotanserin or pharmaceutically acceptable salts, hydrates, polymorphs, or solvates thereof comprises Form I of 1-[3-(4-bromo-2-methyl-2H-pyrazol-3-yl)-4-methoxy-phenyl]-3-(2,4-difluoro-phenyl)-urea, Form II of 1-[3-(4-bromo-2-methyl-2H-pyrazol-3-yl)-4-methoxy-phenyl]-3-(2,4-difluoro-phenyl)-urea or a combination thereof. In some embodiments, the 5-HT2A inverse agonist is administered to a subject in need thereof in an amount that is considered to sub therapeutic.

In some embodiments, the therapeutically effective amount of nelotanserin or pharmaceutically acceptable salts, hydrates, polymorphs, or solvates thereof is configured for immediate release, extended release, delayed release, or any combination thereof.

In some embodiments, the therapeutically effective amount of nelotanserin or pharmaceutically acceptable salts, hydrates, polymorphs, or solvates thereof is from about 0.001 mg to about 1,000 mg, or about 0.001 mg to about 100 mg. In some embodiments, nelotanserin or pharmaceutically acceptable salts, hydrates or solvates thereof is administered to a subject in need thereof in an amount that is considered to sub therapeutic.

In some embodiments, the therapeutically effective amount of nelotanserin or pharmaceutically acceptable salts, hydrates, polymorphs, or solvates thereof is about 20 mg, about 40 mg, or about 80 mg.

In some embodiments, the at least one additional therapeutic agent useful for treating a neurodegenerative disease is a lithium compound or pharmaceutically acceptable salts, hydrates, polymorphs or solvates thereof. In some embodiments, the therapeutically effective amount of a lithium compound or pharmaceutically acceptable salts, hydrates, polymorphs, or solvates thereof is configured for extended release, delayed release, or any combination thereof. In some embodiments, the therapeutically effective amount of a lithium compound or pharmaceutically acceptable salts, hydrates, polymorphs or solvates thereof, is from about 0.001 mg to about 1000 mg, from about 0.001 mg to about 500 mg, from about 0.001 mg to about 100 mg, from about 0.001 mg to about 50 mg, from about 0.001 mg to about 10 mg, from about 0.001 mg to about 1 mg, from about 0.001 mg to about 0.1 mg, or from about 0.001 mg to about 0.01 mg. In some embodiments, the therapeutically effective amount of a lithium compound or pharmaceutically acceptable salts, hydrates, polymorphs or solvates thereof, is about 0.01 mg, about 0.1 mg, about 1 mg, about 5 mg, or about 10 mg. In some embodiments, the lithium compound is present in a sub therapeutic amount. In some embodiments, the sub therapeutic amount of a lithium compound or pharmaceutically acceptable salts, hydrates, polymorphs or solvates thereof, is an amount resulting in a serum concentration of between about 0.4 mM and about 1.6 mM, below about 0.4 mM, below about 0.5 mM, below about 0.4 mM, below about 0.3 mM, below about 0.2 mM, below about 0.1 mM, or below about 0.05 mM when administered to a subject. In some embodiments, the therapeutically effective amount of a lithium compound or pharmaceutically acceptable salts, hydrates, polymorphs, or solvates thereof is configured for extended release, delayed release, or any combination thereof.

In some embodiments, the at least one additional therapeutic agent useful for treating a neurodegenerative disease is levodopa or pharmaceutically acceptable salts, hydrates, polymorphs, or solvates thereof. In some embodiments, the therapeutically effective amount of levodopa or pharmaceutically acceptable salts, hydrates, polymorphs, or solvates thereof is configured for immediate release, extended release, delayed release, or any combination thereof. In some embodiments, the therapeutically effective amount of levodopa or pharmaceutically acceptable salts, hydrates, polymorphs, or solvates thereof is from about 0.001 mg to about 10,000 mg, or about 0.001 mg to about 8,000 mg. In some embodiments, the therapeutically effective amount of levodopa or pharmaceutically acceptable salts, hydrates, polymorphs, or solvates thereof is about 285 mg, about 300 mg, about 400 mg, about 435 mg, 500 mg, about 585 mg, about 600 mg, about 700 mg, about 735 mg, about 750 mg, about 800 mg, about 980 mg, about 1,000 mg, about 1,225 mg, about 1,250 mg, about 1,470 mg, about 1,500 mg, about 1,715 mg, about 1,750 mg, about 1,960 mg, about 2,000 mg, about 2,205 mg, about 2,250 mg, about 2,450 mg, about 2,500 mg, about 2,750 mg, about 3,000 mg, about 3,250 mg, about 3,500 mg, about 3,750 mg, about 4,000 mg, about 4,250 mg, about 5,000 mg, about 5,250 mg, about 5,500 mg, about 5,750 mg, about 6,000 mg, about 6,250 mg, about 6,500 mg, about 6,750 mg, about 7,000 mg, about 7,250 mg, about 7,500 mg, about 7,750 mg, or about 8,000 mg. In some embodiments, the at least one additional therapeutic agent useful for treating a neurodegenerative disease is levodopa or pharmaceutically acceptable salts, hydrates, polymorphs, or solvates thereof and carbidopa or pharmaceutically acceptable salts, hydrates, polymorphs, or solvates thereof. In some embodiments, the therapeutically effective amount of levodopa further comprises carbidopa. In some embodiments, the therapeutically effective amount of carbidopa or pharmaceutically acceptable salts, hydrates, polymorphs, or solvates thereof is configured for immediate release, extended release, delayed release, or any combination thereof. In some embodiments, the therapeutically effective amount of carbidopa is from about 0.001 mg to about 1,000 mg, or from about 0.001 mg to about 700 mg. In some embodiments, the therapeutically effective amount of carbidopa is about 30 mg, about 40 mg, about 50 mg, about 60 mg, about 70 mg, about 71.25 mg, about 80 mg, about 108.75 mg, about 146.25 mg, 183.75 mg, about 245 mg, about 245 mg, about 306.25 mg, about 367.5 mg, about 428.75 mg, about 490 mg, about 551.25 mg, or about 612.5 mg.

In some embodiments, the at least one additional therapeutic agent is an anticonvulsant. In some embodiments, anticonvulsants for use herein may include, but are not limited, to levetiracitam (Keppra), AMPA receptor antagonists, barbiturate anticonvulsants, benzodiazepine anticonvulsants, carbamate anticonvulsants, carbonic anhydrase inhibitor anticonvulsants, dibenzazepine anticonvulsants, fatty acid derivative anticonvulsants, gamma-aminobutyric acid analogs, gamma-aminobutyric acid reuptake inhibitors, hydantoin anticonvulsants, miscellaneous anticonvulsants, neuronal potassium channel openers, oxazolidinedione anticonvulsants, pyrrolidine anticonvulsants, succinimide anticonvulsants, triazine anticonvulsants or combinations thereof. In some embodiments, the anticonvulsant is administered to a subject in need thereof in a therapeutically effective amount. In some embodiments, the anticonvulsant or pharmaceutically acceptable salts, hydrates or solvates thereof is administered to a subject in need thereof in an amount that is considered to sub therapeutic.

In some embodiments, the at least one additional therapeutic agent is a monoclonal antibody. In some embodiments, the second therapeutic agent is a human monoclonal antibody. In some embodiments, the second therapeutic agent is a humanized monoclonal antibody. In some embodiments the monoclonal antibody targets beta amyloid. In some embodiments the beta amyloid may comprise aggregated beta amyloid such as but not limited to soluble oligomers, insoluble fibrils deposited into amyloid plaque, or a combination thereof. In some embodiments, the monoclonal antibody is Aducanumab (BIM037), Gantenerumab, Bapineuzumab, Crenezumab, Ponezumab, Solanezumab, SAR228810, MEDI1814, BAN2401, or any combination thereof. In some embodiments, the monoclonal antibody targets alpha-synuclein. In some embodiments, the monoclonal antibody targeting alpha-synuclein is RG-7935, Posiphen, Affitope PD03A, Affitope PD01A, or any combination thereof.

In some embodiments, the at least one additional therapeutic agent is a BACE enzyme inhibitor. In some embodiments, the BACE enzyme inhibitor is CTS-21166, MK-8931, AZD3293, LY3314814, BI 1181181, LY2886721, E2609, RG7129, JNJ-5486911, TAK-070, or any combination thereof.

In some embodiments, the at least one additional therapeutic agent is a RAGE inhibitor. In some embodiments, the RAGE inhibitor is TTP488 (Azeliragon), TTP4000, FPS-ZM1, or any combination thereof.

In some embodiments, the at least one additional therapeutic agent is an antibody targeting Tau. In some embodiments, the antibody targeting Tau is AADVAC-1, AADVAC-2, ACI-35, BMS-986168, RG7345, TRx-237-015 (LMTX), AV-1451, AV-680, Posiphen, or any combination thereof.

In some embodiments, the at least one additional therapeutic agent is a α7 nicotinic acetylcholine receptor modulator. In some embodiments, the α7 nicotinic acetylcholine receptor modulator is Encenicline (EVP-6124), ABT-126, ABT 418, RG3487, Varenicline, A-867744, TC-5219, AVL3288, BMS933043, DSP-3748, or any combination thereof.

In some embodiments, the at least one additional therapeutic agent may include one or more treatments for Alzheimer's disease such as Namzaric™, Exelon®, Aricept® (donepezil hydrochloride), Namenda® (memantine hydrochloride), or galantamine hydrobromide. In some embodiments, described compositions and formulations may be administered in combination with one or more treatments for Parkinson's Disease such as ABT-126 (Abbott Laboratories), pozanicline (Abbott Laboratories), MABT-5102A (AC Immune), Affitope AD-01 (AFFiRiS GmbH), Affitope AD-02 (AFFiRiS GmbH), davunetide (Allon Therapeutics Inc), nilvadipine derivative (Archer Pharmaceuticals), Anapsos (ASAC Pharmaceutical International AIE), ASP-2535 (Astellas Pharma Inc), ASP-2905 (Astellas Pharma Inc), 1 1C-AZD-2184 (AstraZeneca pic), 1 1C-AZD-2995 (AstraZeneca pic), 18F-AZD-4694 (AstraZeneca pic), AV-965 (Avera Pharmaceuticals Inc), AVN-101 (Avineuro Pharmaceuticals Inc), immune globulin intravenous (Baxter International Inc), EVP-6124 (Bayer AG), nimodipine (Bayer AG), BMS-708163 (Bristol-Myers Squibb Co), CERE-110 (Ceregene Inc), CLL-502 (CLL Pharma), CAD-106 (Cytos Biotechnology AG), mimopezil ((Debiopharm SA), DCB-AD1 (Development Centre for Biotechnology), EGb-761 ((Dr Willmar Schwabe GmbH & Co), E-2012 (Eisai Co Ltd), ACC-001 (Elan Corp pic), bapineuzumab (Elan Corp pic), ELND-006 (Elan Pharmaceuticals Inc), atomoxetine (Eli Lilly & Co), LY-2811376 (Eli Lilly & Co), LY-451395 (Eli Lilly & Co), m266 (Eli Lilly & Co), semagacestat (Eli Lilly & Co), solanezumab (Eli Lilly & Co), AZD-103 (Ellipsis Neurotherapeutics Inc), FGLL (ENKAM Pharmaceuticals A/S), EHT-0202 (ExonHit Therapeutics SA), celecoxib (GD Searle & Co), GSK-933776A (GlaxoSmithKline pic), rosiglitazone XR (GlaxoSmithKline pic), SB-742457 (GlaxoSmithKline pic), R-1578 (Hoffmann-La Roche AG), HF-0220 (Hunter-Fleming Ltd), oxiracetam (ISF Societa Per Azioni), KD-501 (Kwang Dong Pharmaceutical Co Ltd), NGX-267 (Life Science Research Israel), huperzine A (Mayo Foundation), Dimebon (Medivation Inc), MEM-1414 (Memory Pharmaceuticals Corp), MEM-3454 (Memory Pharmaceuticals Corp), MEM-63908 (Memory Pharmaceuticals Corp), MK-0249 (Merck & Co Inc), MK-0752 (Merck & Co Inc), simvastatin (Merck & Co Inc), V-950 (Merck & Co Inc), memantine (Merz & Co GmbH), neramexane (Merz & Co GmbH), Epadel (Mochida Pharmaceutical Co Ltd), 123I-MNI-330 (Molecular Neuroimaging Lie), gantenerumab (MorphoSys AG), NIC5-15 (Mount Sinai School of Medicine), huperzine A (Neuro-Hitech Inc), OXIGON (New York University), NP-12 (Noscira SA), NP-61 (Noscira SA), rivastigmine (Novartis AG), ECT-AD (NsGene A/S), arundic acid (Ono Pharmaceutical Co Ltd), PF-3084014 (Pfizer Inc), PF-3654746 (Pfizer Inc), RQ-00000009 (Pfizer Inc), PYM-50028 (Phytopharm pic), Gero-46 (PN Gerolymatos SA), PBT-2 (Prana Biotechnology Ltd), PRX-03140 (Predix Pharmaceuticals Inc), Exebryl-1 (ProteoTech Inc), PF-4360365 (Rinat Neuroscience Corp), HuCAL anti-beta amyloid monoclonal antibodies (Roche AG), EVT-302 (Roche Holding AG), nilvadipine (Roskamp Institute), galantamine (Sanochemia Pharmazeutika AG), SAR-110894 (sanofi-aventis), INM-176 (Scigenic & Scigen Harvest), mimopezil (Shanghai Institute of Materia Medica of the Chinese Academy of Sciences), NEBO-178 (Stegram Pharmaceuticals), SUVN-502 (Suven Life Sciences), TAK-065 (Takeda Pharmaceutical), ispronicline (Targacept Inc), rasagiline (Teva Pharmaceutical Industries), T-817MA (Toyama Chemical), PF-4494700 (TransTech Pharma Inc), CX-717 (University of California), 18F-FDDNP (University of California Los Angeles), GTS-21 (University of Florida), 18F-AV-133 (University of Michigan), 18F-AV-45 (University of Michigan), tetrathiomolybdate (University of Michigan), 1231-IMPY (University of Pennsylvania), 18F-AV-1/ZK (University of Pennsylvania), 11C-6-Me-BTA-1 (University of Pittsburgh), 18F-6-OH-BTA-1 (University of Pittsburgh), MCD-386 (University of Toledo), leuprolide acetate implant (Voyager Pharmaceutical Corp), aleplasinin (Wyeth), begacestat (Wyeth), GSI-136 (Wyeth), NSA-789 (Wyeth), SAM-531 (Wyeth), CTS-21166 (Zapaq), and ZSET-1446 (Zenyaku Kogyo).

In some embodiments, the at least one additional therapeutic agent may include one or more agents useful for the treatment of motor neuronal disorders, such as AEOL-10150 (Aeolus Pharmaceuticals Inc), riluzole (Aventis Pharma AG), ALS-08 (Avicena Group Inc), creatine (Avicena Group Inc), arimoclomol (Biorex Research and Development Co), mecobalamin (Eisai Co Ltd), talampanel (Eli Lilly & Co), R-7010 (F Hoffmann-La Roche Ltd), edaravone (Mitsubishi-Tokyo Pharmaceuticals Inc), arundic acid (Ono Pharmaceutical Co Ltd), PYM-50018 (Phytopharm pic), RPI-MN (ReceptoPharm Inc), SB-509 (Sangamo Biosciences Inc), olesoxime (Trophos SA), sodium phenylbutyrate (Ucyclyd Pharma Inc), and R-pramipexole (University of Virginia).

In some embodiments, the compositions described herein may include one or more agents known to modify cholinergic transmission such as M1 muscarinic receptor agonists or allosteric modulators, M2 muscarinic antagonists, acetylcholinesterase inhibitors, nicotinic receptor agonists or allosteric modulators, 5-HT4 receptor partial agonists or 5HT1A receptor antagonists and NMDA receptor antagonists or modulators, glutamate antagonists, GABA-ergic antagonists, H3 antagonists, putative metabolic/mitochondrial modulators, or disease modifying agents such as β or γ-secretase inhibitors, Tau-targeted therapeutics, β-amyloid aggregation inhibitors and β-amyloid immunotherapies, an antidepressants, for example a tricyclic, a MAOI (Monoamine oxidase inhibitor) a SSRI (Selective Serotonin Reuptake Inhibitor), a SNRI (Serotonin and Noradrenaline Reuptake Inhibitor) or a NaSSA (noradrenergeric and specific serotonergic antidepressant). Examples of specific antidepressant compounds include amitriptyline, clomipramine, citalopram, dosulepin, doxepin, fluoxetine, imipramine, lofepramine, mirtazapine, moclobemide, nortriptyline, paroxetine, phenelzine, reboxetine, sertraline, tranylcypromine, trazodone, or venlafaxine. In some embodiments, additional therapeutic agents may include antipsychotic drugs, such as olanzapine, clozapine, risperidone, quetiapine, aripiprazole or paliperiden.

Some embodiments are directed to pharmaceutical compositions comprising a therapeutically effective amount of 3-phenylsulfonyl-8-piperazinyl-1yl-quinoline or pharmaceutically acceptable salts, hydrates, polymorphs, or solvates thereof and at least one pharmaceutically acceptable excipient; wherein the therapeutically effective amount of 3-phenylsulfonyl-8-piperazinyl-1yl-quinoline comprises at least one polymorphic form of 3-phenylsulfonyl-8-piperazinyl-1 yl-quinoline. In some embodiments, the at least one polymorphic form is characterized by a powder x-ray diffraction substantially as shown in any one of FIGS. 1-9. In some embodiments, the at least one polymorphic form is characterized by a powder x-ray diffraction substantially as shown in FIG. 2. In some embodiments, the at least one polymorphic form is characterized by a powder x-ray diffraction substantially as shown in FIG. 3. In some embodiments, the at least one polymorphic form is characterized by a powder x-ray diffraction substantially as shown in FIG. 4. In some embodiments, the at least one polymorphic form is characterized by a powder x-ray diffraction substantially as shown in FIG. 5. In some embodiments, the at least one polymorphic form is characterized by a powder x-ray diffraction substantially as shown in FIG. 6. In some embodiments, the at least one polymorphic form is characterized by a powder x-ray diffraction substantially as shown in FIG. 7. In some embodiments, the at least one polymorphic form is characterized by a powder x-ray diffraction substantially as shown in FIG. 8. In some embodiments, the at least one polymorphic form is characterized by a powder x-ray diffraction substantially as shown in FIG. 9.

The therapeutic agents in the methods and compositions described herein may be administered simultaneously or sequentially and, when administration is sequential, either may be administered first, second or third. When administration is simultaneous, the combination may be administered either in the same or different pharmaceutical composition.

The therapeutic agents in the methods and compositions described herein may be used either as separate formulations or as a single combined formulation. In some embodiments, the therapeutic agents in the methods and compositions described herein may be configured into separate formulations. For example, a therapeutically effective amount of 3-phenylsulfonyl-8-piperazinyl-1yl-quinoline or pharmaceutically acceptable salts, hydrates, solvates, or polymorphs, thereof, may be configured in a first composition, a therapeutically effective amount of donepezil may be configured into a second compositions, and a therapeutically effective amount of memantine may be configured into a third composition. In some embodiments, the therapeutic agents in the methods and compositions described herein may be combined into a single formulation. For example, therapeutically effective amounts of 3-phenylsulfonyl-8-piperazinyl-1yl-quinoline or pharmaceutically acceptable salts, hydrates, solvates, or polymorphs, thereof, donepezil, and memantine may be combined into a single composition. In yet other embodiments, the therapeutic agents in the methods and compositions described herein may be configured into multiple separate compositions. For example, a therapeutically effective amount of 3-phenylsulfonyl-8-piperazinyl-1yl-quinoline or pharmaceutically acceptable salts, hydrates, solvates, or polymorphs, thereof, maybe be formulated into a first composition and therapeutically effective amounts of donepezil and memantine may be formulated into a second formulation. Alternatively, a therapeutically effective amount of 3-phenylsulfonyl-8-piperazinyl-1yl-quinoline or pharmaceutically acceptable salts, hydrates, solvates, or polymorphs, thereof, may be combined with a therapeutically effective amount of donepezil into a first composition and a therapeutically effective amount of memantine may be configured into a second composition. Alternatively, a therapeutically effective amount of 3-phenylsulfonyl-8-piperazinyl-1yl-quinoline or pharmaceutically acceptable salts, hydrates, solvates, or polymorphs, thereof, may be combined with a therapeutically effective amount of memantine into a first composition and a therapeutically effective amount of donepezil may be configured into a second composition. When combined in the same formulation, it will be appreciated that the compounds must be stable and compatible with each other and the other components of the formulation.

In some embodiments, the at least one pharmaceutically acceptable excipient is selected from the group consisting of microcrystalline cellulose, mannitol, sodium starch glycolate, hydroxypropyl methylcellulose, purified water, magnesium stearate, croscarmellose sodium, a glue, and any combination thereof.

When the compounds of this invention are administered in combination therapies with other agents, they may be administered sequentially or concurrently to the patient. Alternatively, pharmaceutical or prophylactic compositions according to this invention comprise a therapeutically effective amount of 3-phenylsulfonyl-8-piperazinyl-1yl-quinoline or pharmaceutically acceptable salts, hydrates, polymorphs or solvates thereof, and a therapeutically effective amount of at least one additional therapeutic agent. Additional therapeutic agents that are normally administered to treat a particular disease or condition may be referred to as “agents appropriate for the disease, or condition, being treated.”

If pharmaceutically acceptable salts of the compounds of this invention are utilized in these compositions, those salts are preferably derived from inorganic or organic acids and bases. Included among such acid salts are the following: acetate, adipate, alginate, aspartate, benzoate, benzene sulfonate, bisulfate, butyrate, citrate, camphorate, camphor sulfonate, cyclopentanepropionate, digluconate, dodecyl sulfate, ethanesulfonate, fumarate, glucoheptanoate, glycerophosphate, hemisulfate, heptanoate, hexanoate, hydrochloride, hydrobromide, hydroiodide, 2-hydroxyethanesulfonate, lactate, maleate, methanesulfonate, 2-naphthalenesulfonate, nicotinate, oxalate, pamoate, pectinate, persulfate, 3-phenyl-propionate, picrate, pivalate, propionate, succinate, tartrate, thiocyanate, tosylate and undecanoate. Base salts include ammonium salts, alkali metal salts, such as sodium and potassium salts, alkaline earth metal salts, such as calcium and magnesium salts, salts with organic bases, such as dicyclohexylamine salts, N-methyl-D-glucamine, and salts with amino acids such as arginine, lysine, and so forth.

Also, the basic nitrogen-containing groups can be quaternized with such agents as lower alkyl halides, such as methyl, ethyl, propyl, and butyl chlorides, bromides and iodides; dialkyl sulfates, such as dimethyl, diethyl, dibutyl and diamyl sulfates; long chain halides such as decyl, lauryl, myristyl and stearyl chlorides, bromides and iodides; aralkyl halides, such as benzyl and phenethyl bromides and others. Water or oil-soluble or dispersible products are thereby obtained.

The compounds utilized in the compositions and methods of this invention may also be modified by appending appropriate functionalities to enhance selective biological properties. Such modifications are known in the art and include those, which increase biological penetration into a given biological system (e.g., blood, lymphatic system, or central nervous system), increase oral availability, increase solubility to allow administration by injection, alter metabolism and/or alter rate of excretion.

According to a preferred embodiment, the compositions of this invention are formulated for pharmaceutical administration to a subject or patient, e.g., a mammal, preferably a human being. Such pharmaceutical compositions are used to ameliorate, treat or prevent any of the diseases described herein including but not limited to neurodegenerative diseases in a subject.

Agents of the invention are often administered as pharmaceutical compositions comprising an active therapeutic agent, i.e., and a variety of other pharmaceutically acceptable components. See Remington's Pharmaceutical Sciences (19th Edition (Mack Publishing Company, 1995)). The preferred form depends on the intended mode of administration and therapeutic application. The compositions can also include, depending on the formulation desired, pharmaceutically acceptable, non-toxic carriers or diluents, which are defined as vehicles commonly used to formulate pharmaceutical compositions for animal or human administration. The diluent is selected so as not to affect the biological activity of the combination. Examples of such diluents are distilled water, physiological phosphate-buffered saline, Ringer's solutions, dextrose solution, and Hank's solution. In addition, the pharmaceutical composition or formulation may also include other carriers, adjuvants, or nontoxic, nontherapeutic, nonimmunogenic stabilizers and the like.

In some embodiments, the present invention provides pharmaceutically acceptable compositions comprising a therapeutically effective amount of one or more of a described compound, formulated together with one or more pharmaceutically acceptable excipients including but not limited to, carriers (additives) and/or diluents for use in treating the diseases described herein, including, but not limited to a neurodegenerative disease. While it is possible for a described compound to be administered alone, it is preferable to administer a described compound as a pharmaceutical formulation (composition) as described herein. Described compounds may be formulated for administration in any convenient way for use in human or veterinary medicine, by analogy with other pharmaceuticals.

As described in detail, pharmaceutical compositions of the present invention may be specially formulated for administration in solid or liquid form, including those adapted for the following: oral administration, for example, drenches (aqueous or non-aqueous solutions or suspensions), tablets, e.g., those targeted for buccal, sublingual, and systemic absorption, boluses, powders, granules, pastes for application to the tongue; parenteral administration, for example, by subcutaneous, intramuscular, intravenous or epidural injection as, for example, a sterile solution or suspension, or delayed-release formulation; topical application, for example, as a cream, ointment, or a controlled-release patch or spray applied to the skin, lungs, or oral cavity; intravaginally or intrarectally, for example, as a pessary, cream or foam; sublingually; ocularly; transdermally; or nasally, pulmonary and to other mucosal surfaces.

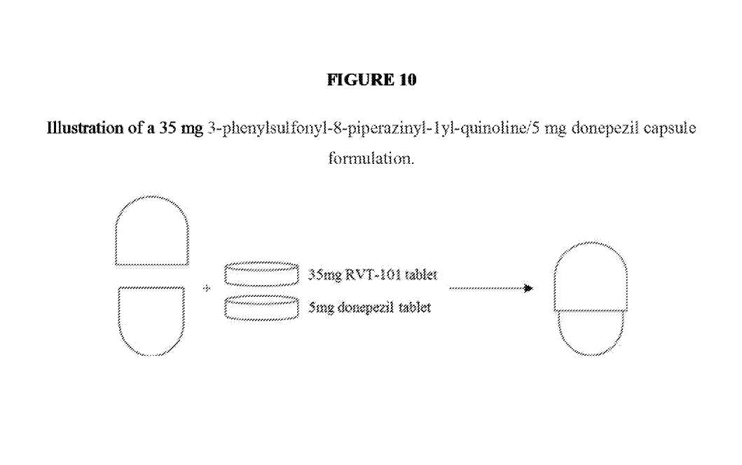

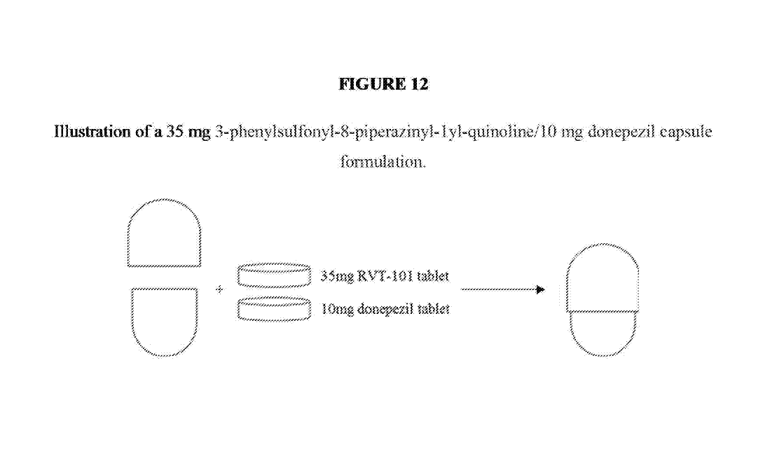

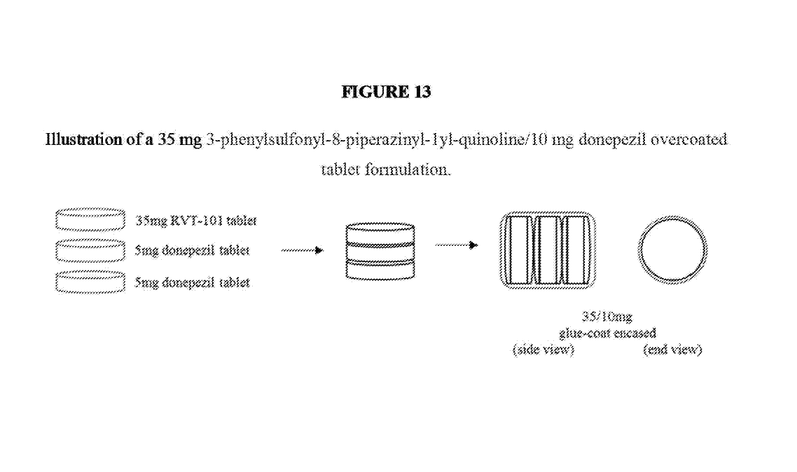

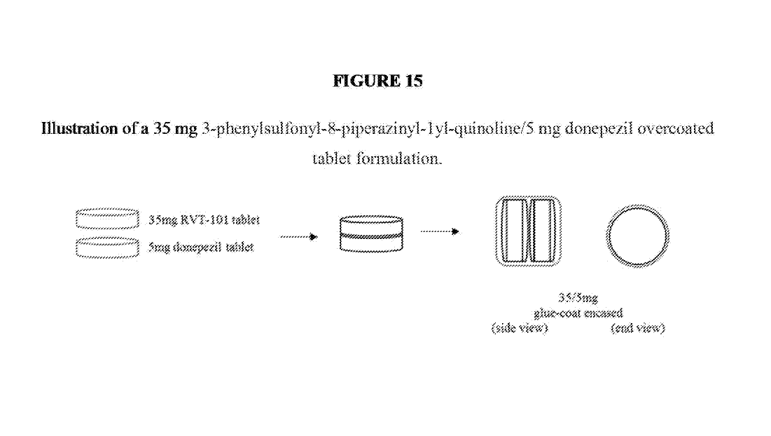

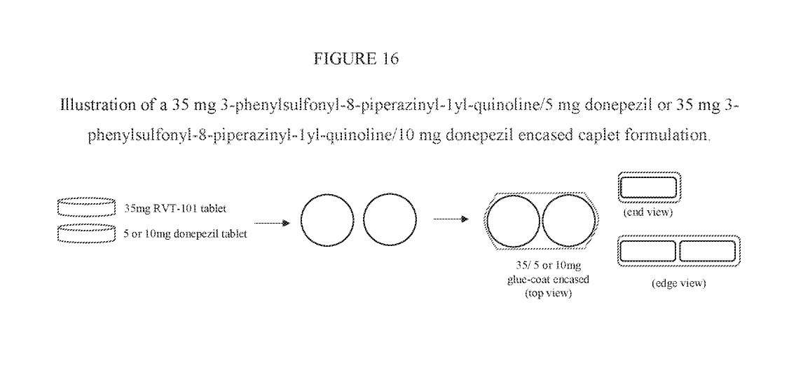

In some embodiments, the compositions described herein can be configured as overcoated tablet formulations such as, but not limited to, those shown in FIGS. 10-15. In some embodiments, the compositions described herein can be configured as an encased product coated edge-to-edge tablet formulations such as the example shown in FIG. 16. In some embodiments, a flat-oval edge-to-edge formulation might also be obtained from a hard-gelatin or HPMC capsule manufactured using a flattened mold rather than a circular mold. In some embodiments a “flattened” capsule would be a more desirable alternative to the standard circular capsule.

Pharmaceutically acceptable salts of compounds described herein include conventional nontoxic salts or quaternary ammonium salts of a compound, e.g., from non-toxic organic or inorganic acids. For example, such conventional nontoxic salts include those derived from inorganic acids such as hydrochloride, hydrobromic, sulfuric, sulfamic, phosphoric, nitric, and the like; and the salts prepared from organic acids such as acetic, propionic, succinic, glycolic, stearic, lactic, malic, tartaric, citric, ascorbic, palmitic, maleic, hydroxymaleic, phenyl acetic, glutamic, benzoic, salicyclic, sulfanilic, 2-acetoxybenzoic, fumaric, toluenesulfonic, methanesulfonic, ethane disulfonic, oxalic, isothionic, and the like. In other cases, described compounds may contain one or more acidic functional groups and, thus, are capable of forming pharmaceutically acceptable salts with pharmaceutically acceptable bases. These salts can likewise be prepared in situ in the administration vehicle or the dosage form manufacturing process, or by separately reacting the purified compound in its free acid form with a suitable base, such as the hydroxide, carbonate or bicarbonate of a pharmaceutically acceptable metal cation, with ammonia, or with a pharmaceutically acceptable organic primary, secondary or tertiary amine. Representative alkali or alkaline earth salts include the lithium, sodium, potassium, calcium, magnesium, and aluminum salts and the like. Representative organic amines useful for the formation of base addition salts include ethylamine, diethylamine, ethylenediamine, ethanolamine, diethanolamine, piperazine and the like.

Wetting agents, emulsifiers and lubricants, such as sodium lauryl sulfate and magnesium stearate, as well as coloring agents, release agents, coating agents, sweetening, flavoring and perfuming agents, preservatives and antioxidants can also be present in the compositions.

Examples of pharmaceutically acceptable antioxidants include: water soluble antioxidants, such as ascorbic acid, cysteine hydrochloride, sodium bisulfate, sodium metabisulfite, sodium sulfite and the like; oil-soluble antioxidants, such as ascorbyl palmitate, butylated hydroxyanisole (BHA), butylated hydroxytoluene (BHT), lecithin, propyl gallate, alpha-tocopherol, and the like; and metal chelating agents, such as citric acid, ethylenediamine tetraacetic acid (EDTA), sorbitol, tartaric acid, phosphoric acid, and the like.

Formulations for use in accordance with the present invention include those suitable for oral, nasal, topical (including buccal and sublingual), rectal, vaginal and/or parenteral administration. The formulations may conveniently be presented in unit dosage form and may be prepared by any methods well known in the art of pharmacy. The amount of active ingredient, which can be combined with a carrier material to produce a single dosage form will vary depending upon the host being treated, and the particular mode of administration. The amount of active ingredient that can be combined with a carrier material to produce a single dosage form will generally be that amount of the compound, which produces a therapeutic effect. Generally, this amount will range from about 1% to about 99% of active ingredient, preferably from about 5% to about 70%, most preferably from about 10% to about 30%.

In certain embodiments, a formulation as described herein comprises an excipient selected from the group consisting of cyclodextrins, liposomes, micelle forming agents, e.g., bile acids, and polymeric carriers, e.g., polyesters and polyanhydrides; and a compound of the present invention. In certain embodiments, an aforementioned formulation renders orally bioavailable a described compound of the present invention.

The compositions described herein optionally contain inactive carriers and diluents known to one of skill in the art such as, for example microcrystalline cellulose (10-150 mg), mannitol (10-100 mg), sodium starch glycolate (0.001-20 mg, or 1-20 mg), hydroxypropyl methylcellulose (1-20 mg), magnesium stearate (1-10 mg), and purified water.

Methods of preparing formulations or compositions comprising described compounds include a step of bringing into association a compound of the present invention with the carrier and, optionally, one or more accessory ingredients (excipients). In general, formulations may be prepared by uniformly and intimately bringing into association a compound of the present invention with liquid carriers, or finely divided solid carriers, or both, and then, if necessary, shaping the product.

The pharmaceutical compositions may be in the form of a sterile injectable preparation, for example, as a sterile injectable aqueous or oleaginous suspension. This suspension may be formulated according to techniques known in the art using suitable dispersing or wetting agents (such as, for example, Tween 80) and suspending agents. The sterile injectable preparation may also be a sterile injectable solution or suspension in a non-toxic parenterally acceptable diluent or solvent, for example, as a solution in 1,3-butanediol. Among the acceptable vehicles and solvents that may be employed are mannitol, water, Ringer's solution and isotonic sodium chloride solution. In addition, sterile, fixed oils are conventionally employed as a solvent or suspending medium. For this purpose, any bland fixed oil may be employed including synthetic mono- or diglycerides. Fatty acids, such as oleic acid and its glyceride derivatives are useful in the preparation of injectables, as are natural pharmaceutically acceptable oils, such as olive oil or castor oil, especially in their polyoxyethylated versions. These oil solutions or suspensions may also contain a long-chain alcohol diluent or dispersant, such as those described in Pharmacopeia Helvetica, or a similar alcohol. Other commonly used surfactants, such as Tweens, Spans and other emulsifying agents or bioavailability enhancers which are commonly used in the manufacture of pharmaceutically acceptable solid, liquid, or other dosage forms may also be used for the purposes of formulation.

In some cases, in order to prolong the effect of a drug, it may be desirable to slow the absorption of the drug from subcutaneous or intramuscular injection. This may be accomplished by the use of a liquid suspension of crystalline or amorphous material having poor water solubility. The rate of absorption of the drug then depends upon its rate of dissolution, which in turn, may depend upon crystal size and crystalline form. Alternatively, delayed absorption of a parenterally administered drug form is accomplished by dissolving or suspending the drug in an oil vehicle.

Injectable depot forms are made by forming microencapsule matrices of the described compounds in biodegradable polymers such as polylactide-polyglycolide. Depending on the ratio of drug to polymer, and the nature of the particular polymer employed, the rate of drug release can be controlled. Examples of other biodegradable polymers include poly(orthoesters) and poly(anhydrides). Depot injectable formulations are also prepared by entrapping the drug in liposomes or microemulsions, which are compatible with body tissue.

The pharmaceutical compositions of this invention may be orally administered in any orally acceptable dosage form including, but not limited to, capsules, tablets, and aqueous suspensions and solutions. In the case of tablets for oral use, carriers, which are commonly used include but are not limited to lactose and cellulose (carboxymethylcellulose). Lubricating agents, such as magnesium stearate, are also typically added. For oral administration in a capsule form, useful diluents include but are not limited to lactose and cellulose (carboxymethylcellulose). When aqueous suspensions and solutions and propylene glycol are administered orally, the active ingredient is combined with emulsifying and suspending agents. If desired, certain sweetening and/or flavoring and/or coloring agents may be added.

Formulations described herein suitable for oral administration may be in the form of capsules, cachets, pills, tablets, lozenges (using a flavored basis, usually sucrose and acacia or tragacanth), powders, granules, or as a solution or a suspension in an aqueous or non-aqueous liquid, or as an oil-in-water or water-in-oil liquid emulsion, or as an elixir or syrup, or as pastilles (using an inert base, such as gelatin and glycerin, or sucrose and acacia) and/or as mouth washes and the like, each containing a predetermined amount of a compound of the present invention as an active ingredient. Compounds described herein may also be administered as a bolus, electuary or paste.

In solid dosage forms for oral administration (capsules, tablets, pills, dragees, powders, granules and the like), an active ingredient is mixed with one or more pharmaceutically-acceptable carriers, such as sodium citrate or dicalcium phosphate, and/or any of the following: fillers or extenders, such as starches, lactose, sucrose, glucose, mannitol, and/or silicic acid; binders, such as, for example, carboxymethylcellulose, alginates, gelatin, polyvinyl pyrrolidone, sucrose and/or acacia; humectants, such as glycerol; disintegrating agents, such as agar-agar, calcium carbonate, potato or tapioca starch, alginic acid, certain silicates, and sodium carbonate; solution retarding agents, such as paraffin; absorption accelerators, such as quaternary ammonium compounds; wetting agents, such as, for example, cetyl alcohol, glycerol monostearate, and non-ionic surfactants; absorbents, such as kaolin and bentonite clay; lubricants, such as talc, calcium stearate, magnesium stearate, solid polyethylene glycols, sodium lauryl sulfate, and mixtures thereof; and coloring agents. In the case of capsules, tablets and pills, the pharmaceutical compositions may also comprise buffering agents. Solid compositions of a similar type may also be employed as fillers in soft and hard-shelled gelatin capsules using such excipients as lactose or milk sugars, as well as high molecular weight polyethylene glycols and the like.

Tablets may be made by compression or molding, optionally with one or more accessory ingredients (excipients). Compressed tablets may be prepared using binder (for example, gelatin or hydroxypropylmethyl cellulose), lubricant, inert diluent, preservative, disintegrant (for example, sodium starch glycolate or cross-linked sodium carboxymethyl cellulose), surface-active or dispersing agent. Molded tablets may be made in a suitable machine in which a mixture of the powdered compound is moistened with an inert liquid diluent. If a solid carrier is used, the preparation can be in tablet form, placed in a hard gelatin capsule in powder or pellet form, or in the form of a troche or lozenge. The amount of solid carrier will vary, e.g., from about 0.01 to 800 mg, preferably about 0.01 mg to 400 mg, about or 3 mg to about 400 mg. When a liquid carrier is used, the preparation can be, e.g., in the form of a syrup, emulsion, soft gelatin capsule, sterile injectable liquid such as an ampule or nonaqueous liquid suspension. Where the composition is in the form of a capsule, any routine encapsulation is suitable, for example, using the aforementioned carriers in a hard gelatin capsule shell.

Tablets and other solid dosage forms, such as dragees, capsules, pills and granules, may optionally be scored or prepared with coatings and shells, such as enteric coatings and other coatings well known in the pharmaceutical-formulating art. They may alternatively or additionally be formulated so as to provide slow or controlled release of the active ingredient therein using, for example, hydroxypropylmethyl cellulose in varying proportions to provide the desired release profile, other polymer matrices, liposomes and/or microspheres. They may be formulated for rapid release, e.g., freeze-dried. They may be sterilized by, for example, filtration through a bacteria-retaining filter, or by incorporating sterilizing agents in the form of sterile solid compositions that can be dissolved in sterile water, or some other sterile injectable medium immediately before use. These compositions may also optionally contain opacifying agents and may be of a composition that they release the active ingredient(s) only, or preferentially, in a certain portion of the gastrointestinal tract, optionally, in a delayed manner. Examples of embedding compositions that can be used include polymeric substances and waxes. The active ingredient can also be in micro-encapsulated form, if appropriate, with one or more of the above-described excipients.

Liquid dosage forms for oral administration of compounds of the invention include pharmaceutically acceptable emulsions, microemulsions, solutions, suspensions, syrups and elixirs. In addition to the active ingredient, the liquid dosage forms may contain inert diluents commonly used in the art, such as, for example, water or other solvents, solubilizing agents and emulsifiers, such as ethyl alcohol, isopropyl alcohol, ethyl carbonate, ethyl acetate, benzyl alcohol, benzyl benzoate, propylene glycol, 1,3-butylene glycol, oils (in particular, cottonseed, groundnut, corn, germ, olive, castor and sesame oils), glycerol, tetrahydrofuryl alcohol, polyethylene glycols and fatty acid esters of sorbitan, and mixtures thereof.

Besides inert diluents, oral compositions can also include adjuvants such as wetting agents, emulsifying and suspending agents, sweetening, flavoring, coloring, perfuming and preservative agents.

Suspensions, in addition to active compounds, may contain suspending agents as, for example, ethoxylated isostearyl alcohols, polyoxyethylene sorbitol and sorbitan esters, microcrystalline cellulose, aluminum metahydroxide, bentonite, agar-agar and tragacanth, and mixtures thereof.

The pharmaceutical compositions of this invention may also be administered in the form of suppositories for rectal administration. These compositions can be prepared by mixing a compound of this invention with a suitable non-irritating excipient, which is solid at room temperature but liquid at the rectal temperature and therefore will melt in the rectum to release the active components. Such materials include, but are not limited to, cocoa butter, beeswax and polyethylene glycols.

Topical administration of the pharmaceutical compositions of this invention is especially useful when the desired treatment involves areas or organs readily accessible by topical application. For application topically to the skin, the pharmaceutical composition should be formulated with a suitable ointment containing the active components suspended or dissolved in a carrier. Carriers for topical administration of the compounds of this invention include, but are not limited to, mineral oil, liquid petroleum, white petroleum, propylene glycol, polyoxyethylene polyoxypropylene compound, emulsifying wax and water. Alternatively, the pharmaceutical composition can be formulated with a suitable lotion or cream containing the active compound suspended or dissolved in a carrier. Suitable carriers include, but are not limited to, mineral oil, sorbitan monostearate, polysorbate 60, cetyl esters wax, cetearyl alcohol, 2-octyldodecanol, benzyl alcohol and water. The pharmaceutical compositions of this invention may also be topically applied to the lower intestinal tract by rectal suppository formulation or in a suitable enema formulation. Topically-administered transdermal patches are also included in this invention. Transdermal patches have the added advantage of providing controlled delivery of a compound of the present invention to the body. Dissolving or dispersing the compound in the proper medium can make such dosage forms. Absorption enhancers can also be used to increase the flux of the compound across the skin. Either providing a rate controlling membrane or dispersing the compound in a polymer matrix or gel can control the rate of such flux.

The pharmaceutical compositions of this invention may be administered by nasal aerosol or inhalation. Such compositions are prepared according to techniques well-known in the art of pharmaceutical formulation and may be prepared as solutions in saline, employing benzyl alcohol or other suitable preservatives, absorption promoters to enhance bioavailability, fluorocarbons, and/or other solubilizing or dispersing agents known in the art.

For ophthalmic use, the pharmaceutical compositions may be formulated as micronized suspensions in isotonic, pH adjusted sterile saline, or, preferably, as solutions in isotonic, pH adjusted sterile saline, either with or without a preservative such as benzylalkonium chloride. Alternatively, for ophthalmic uses, the pharmaceutical compositions may be formulated in an ointment such as petrolatum.

Examples of suitable aqueous and nonaqueous carriers, which may be employed in the pharmaceutical compositions of the invention, include water, ethanol, polyols (such as glycerol, propylene glycol, polyethylene glycol, and the like), and suitable mixtures thereof, vegetable oils, such as olive oil, and injectable organic esters, such as ethyl oleate. Proper fluidity can be maintained, for example, by the use of coating materials, such as lecithin, by the maintenance of the required particle size in the case of dispersions, and by the use of surfactants.

Such compositions may also contain adjuvants such as preservatives, wetting agents, emulsifying agents and dispersing agents. Inclusion of one or more antibacterial and/or antifungal agents, for example, paraben, chlorobutanol, phenol sorbic acid, and the like, may be desirable in certain embodiments. It may alternatively or additionally be desirable to include isotonic agents, such as sugars, sodium chloride, and the like into the compositions. In addition, prolonged absorption of the injectable pharmaceutical form may be brought about by the inclusion of agents, which delay absorption such as aluminum monostearate and gelatin.

In certain embodiments, a described compound or pharmaceutical preparation is administered orally. In other embodiments, a described compound or pharmaceutical preparation is administered intravenously. Alternative routes of administration include sublingual, intramuscular, and transdermal administrations.

When compounds described herein are administered as pharmaceuticals, to humans and animals, they can be given per se or as a pharmaceutical composition containing, for example, 0.1% to 99.5% (more preferably, 0.5% to 90%) of active ingredient in combination with a pharmaceutically acceptable carrier.

Preparations described herein may be given orally, parenterally, topically, or rectally. They are of course given in forms suitable for the relevant administration route. For example, they are administered in tablets or capsule form, by injection, inhalation, eye lotion, ointment, suppository, etc. administration by injection, infusion or inhalation; topical by lotion or ointment; and rectal by suppositories. Oral administrations are preferred.

Such compounds may be administered to humans and other animals for therapy by any suitable route of administration, including orally, nasally, as by, for example, a spray, rectally, intravaginally, parenterally, intracisternally and topically, as by powders, ointments or drops, including buccally and sublingually.

Regardless of the route of administration selected, compounds described herein which may be used in a suitable hydrated form, and/or the pharmaceutical compositions of the present invention, are formulated into pharmaceutically-acceptable dosage forms by conventional methods known to those of skill in the art.

Actual dosage levels of the active ingredients in the pharmaceutical compositions of the invention may be varied so as to obtain an amount of the active ingredient that is effective to achieve the desired therapeutic response for a particular patient, composition, and mode of administration, without being toxic to the patient.

The pharmaceutical compositions described herein may be prepared by admixture, suitably at ambient temperature and atmospheric pressure, and is usually adapted for oral, parenteral or rectal administration and, as such, may be in the form of tablets, capsules, oral liquid preparations, powders, granules, lozenges, reconstitutable powders, injectable or infusible solutions or suspensions or suppositories. Orally administrable compositions are generally preferred.

Tablets and capsules for oral administration may be in unit dose form, and may contain conventional excipients, such as binding agents, fillers, tableting lubricants, disintegrants and acceptable wetting agents. The tablets may be coated according to methods well known in normal pharmaceutical practice.

Oral liquid preparations may be in the form of, for example, aqueous or oily suspension, solutions, emulsions, syrups or elixirs, or may be in the form of a dry product for reconstitution with water or other suitable vehicle before use. Such liquid preparations may contain conventional additives such as suspending agents, emulsifying agents, non-aqueous vehicles (which may include edible oils), preservatives, and, if desired, conventional flavorings or colorants.

For parenteral administration, fluid unit dosage forms are prepared utilizing a compound and a sterile vehicle. The compound, depending on the vehicle and concentration used, can be either suspended or dissolved in the vehicle. In preparing solutions, the compound can be dissolved for injection and filter sterilized before filling into a suitable vial or ampoule and sealing. Advantageously, adjuvants such as a local anesthetic, preservatives and buffering agents are dissolved in the vehicle. To enhance the stability, the composition can be frozen after filling into the vial and the water removed under vacuum. Parenteral suspensions are prepared in substantially the same manner, except that the compound is suspended in the vehicle instead of being dissolved, and sterilization cannot be accomplished by filtration. The compound can be sterilized by exposure to ethylene oxide before suspension in a sterile vehicle. Advantageously, a surfactant or wetting agent is included in the composition to facilitate uniform distribution of the compound.

The compositions comprising a therapeutically effective amount of 3-phenylsulfonyl-8-piperazinyl-1yl-quinoline or pharmaceutically acceptable salts, hydrates, polymorphs, or solvates thereof and a therapeutically effective amount of at least one additional therapeutic agent useful for treating a neurodegenerative disease, used in the treatment of a neurodegenerative disease will vary in the usual way with the seriousness of the disorders, the weight of the sufferer, and other similar factors. However, as a general guide, such unit doses will preferably be administered once a day, although administration more than once a day may be required; and such therapy may extend for a number of weeks or months.

The composition may contain from 0.1% to 99% by weight, preferably from 10 to 60% by weight, of the active material, depending on the method of administration.

Compositions may, if desired, be presented in a pack or dispenser device which may contain one or more unit dosage forms containing the active ingredients. The pack may, for example, comprise metal or plastic foil, such as a blister pack. Where the compounds are intended for administration as two separate compositions these may be presented, for example, in the form of a twin pack.

Pharmaceutical compositions may also be prescribed to the patient in “patient packs” containing the whole course of treatment in a single package, usually a blister pack. Patient packs have an advantage over traditional prescriptions, where a pharmacist divides a patient's supply of a pharmaceutical from a bulk supply, in that the patient always has access to the package insert contained in the patient pack, normally missing in traditional prescriptions. The inclusion of a package insert has been shown to improve patient compliance with the physician's instructions.

It will be understood that the administration of the combination by means of a single patient pack, or patient packs of each composition, including a package insert directing the patient to the correct use of the combination is a desirable additional embodiment. Some embodiments are directed to a patient pack comprising at least one active ingredient, of the combination and an information insert containing directions on the use of the combination. Some embodiments are directed to a double pack comprising in association for separate administration of a therapeutically effective amount of 3-phenylsulfonyl-8-piperazinyl-1yl-quinoline or pharmaceutically acceptable salts, hydrates, polymorphs, or solvates thereof and a therapeutically effective amount of at least one additional therapeutic agent useful for treating a neurodegenerative disease.

The therapeutically effective amount of 3-phenylsulfonyl-8-piperazinyl-1yl-quinoline or pharmaceutically acceptable salts, hydrates, polymorphs, or solvates thereof, used in the treatment of a neurodegenerative disease will vary in the usual way with the seriousness of the disorders, the weight of the sufferer, and other similar factors. However, as a general guide, suitable unit doses may be about 0.001 to about 1,000 mg, more suitably 0.001 to 200 mg, for example about 20 to about 40 mg; about 35 mg, or about 70 mg, and such unit doses will preferably be administered once a day, although administration more than once a day may be required; and such therapy may extend for a number of weeks or months.

The therapeutically effective amount of 3-phenylsulfonyl-8-piperazinyl-1yl-quinoline or pharmaceutically acceptable salts, hydrates, polymorphs, or solvates thereof, used in combination with at least one additional therapeutic agent useful for treating a neurodegenerative disease may be the same as when it is used on its own or may be different. In a particular embodiment, it may be possible that the dose of either drug used may be higher when used in combination than when used separately. In a particular embodiment, the therapeutically effective amount of 3-phenylsulfonyl-8-piperazinyl-1yl-quinoline or pharmaceutically acceptable salts, hydrates, polymorphs, or solvates thereof, may be increased, may be the same, or may be decreased when combined with at least one additional therapeutic agent useful for treating a neurodegenerative disease.

The dose when using the compounds of the present invention can vary within wide limits, and as is customary and is known to the physician, it is to be tailored to the individual conditions in each individual case. It depends, for example, on the nature and severity of the illness to be treated, on the condition of the patient, on the compound employed or on whether an acute or chronic disease state is treated or prophylaxis is conducted or on whether further active compounds are administered in addition to the compounds of the present invention. Representative doses of the present invention include, but are not limited to, about 0.001 mg to about 5,000 mg, about 0.001 mg to about 2,500 mg, about 0.001 mg to about 1,000 mg, 0.001 mg to about 500 mg, 0.001 mg to about 250 mg, about 0.001 mg to 175 mg, about 0.001 mg to 100 mg, about 0.001 mg to 70 mg, about 0.001 mg to about 50 mg, and about 0.001 mg to about 35 mg. Multiple doses may be administered during the day, especially when relatively large amounts are deemed to be needed, for example 2, 3, or 4, doses. Depending on the individual and as deemed appropriate from the patient's physician or care-giver it may be necessary to deviate upward or downward from the doses described herein.

The amount of active ingredient, or an active salt or derivative thereof, required for use in treatment will vary not only with the particular salt selected but also with the route of administration, the nature of the condition being treated and the age and condition of the patient and will ultimately be at the discretion of the attendant physician or clinician. In general, one skilled in the art understands how to extrapolate in vivo data obtained in a model system, typically an animal model, to another, such as a human. In some circumstances, these extrapolations may merely be based on the weight of the animal model in comparison to another, such as a mammal, preferably a human, however, more often, these extrapolations are not simply based on weights, but rather incorporate a variety of factors. Representative factors include the type, age, weight, sex, diet and medical condition of the patient, the severity of the disease, the route of administration, pharmacological considerations such as the activity, efficacy, pharmacokinetic and toxicology profiles of the particular compound employed, whether a drug delivery system is utilized, on whether an acute or chronic disease state is being treated or prophylaxis is conducted or on whether further active compounds are administered in addition to the compounds of the present invention and as part of a drug combination. The dosage regimen for treating a disease condition with the compounds and/or compositions of this invention is selected in accordance with a variety factors as cited above. Thus, the actual dosage regimen employed may vary widely and therefore may deviate from a preferred dosage regimen and one skilled in the art will recognize that dosage and dosage regimen outside these typical ranges can be tested and, where appropriate, may be used in the methods of this invention.

The desired dose may conveniently be presented in a single dose or as divided doses administered at appropriate intervals, for example, as two, three, four or more sub-doses per day. The sub-dose itself may be further divided, e.g., into a number of discrete loosely spaced administrations. The daily dose can be divided, especially when relatively large amounts are administered as deemed appropriate, into several, for example 2, 3, or 4, part administrations. If appropriate, depending on individual behavior, it may be necessary to deviate upward or downward from the daily dose indicated.

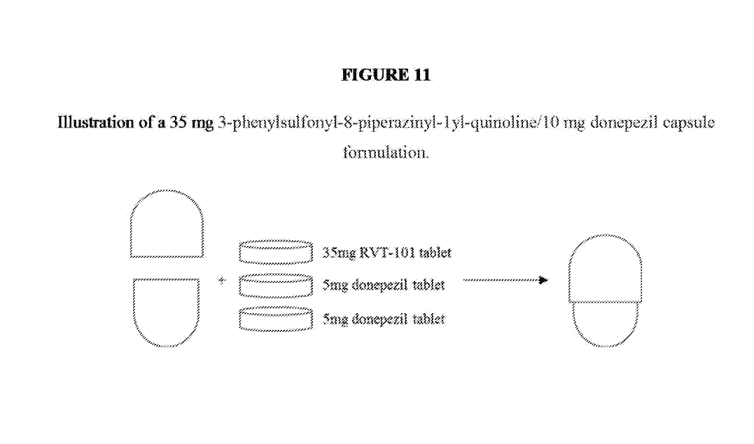

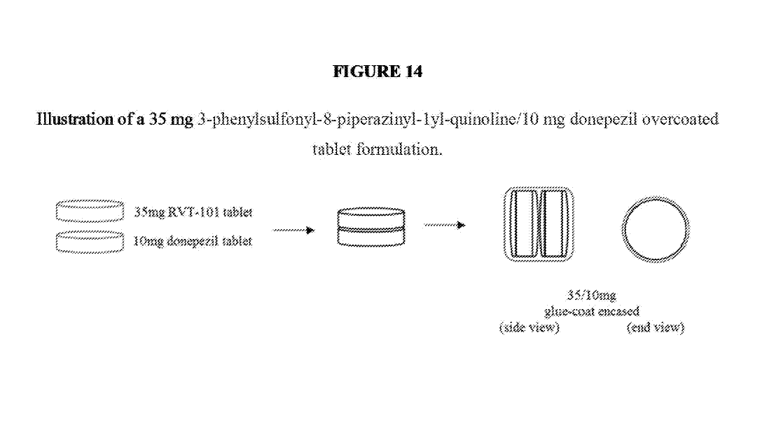

The Tablets of FIGS. 14-16 and other solid dosage forms, such as dragees, capsules of FIGS. 11-13, pills and granules, may optionally be scored or prepared with coatings and shells, such as enteric coatings and other coatings well known in the pharmaceutical-formulating art. They may alternatively or additionally be formulated so as to provide slow or controlled release of the active ingredient therein using, for example, hydroxypropylmethyl cellulose in varying proportions to provide the desired release profile, other polymer matrices, liposomes and/or microspheres. They may be formulated for rapid release, e.g., freeze-dried. They may be sterilized by, for example, filtration through a bacteria-retaining filter, or by incorporating sterilizing agents in the form of sterile solid compositions that can be dissolved in sterile water, or some other sterile injectable medium immediately before use. These compositions may also optionally contain opacifying agents and may be of a composition that they release the active ingredient(s) only, or preferentially, in a certain portion of the gastrointestinal tract, optionally, in a delayed manner. Examples of embedding compositions that can be used include polymeric substances and waxes. The active ingredient can also be in micro-encapsulated form, if appropriate, with one or more of the above-described excipients.

The compositions described herein may be useful in the treatment and prophylaxis of a neurodegenerative disease. In some embodiments, the neurodegenerative disease is selected from Alzheimer's disease (including mild or early-stage Alzheimer's disease, mild to moderate Alzheimer's disease, moderate or mid-stage Alzheimer's disease, moderate to severe Alzheimer's disease, moderately severe Alzheimer's disease, severe Alzheimer's disease, Alzheimer's disease with Lewy bodies, (AD)), Parkinson's disease (including Parkinson's disease chemically induced by exposure to environmental agents such as pesticides, insecticides, or herbicides and/or metals such as manganese, aluminum, cadmium, copper, or zinc, SNCA gene-linked Parkinson's disease, sporadic or idiopathic Parkinson's disease, or Parkin- or LRRK2-linked Parkinson's disease (PD)), autosomal-dominant Parkinson's disease, Diffuse Lewy Body Disease (DLBD) also known as Dementia with Lewy Bodies (DLB), Pure Autonomic Failure, Lewy body dysphagia, Incidental LBD, Inherited LBD (e.g., mutations of the alpha-synuclein gene, PARK3 and PARK4), multiple system atrophy (including Olivopontocerebellar Atrophy, Striatonigral Degeneration, Shy-Drager Syndrome (MSA)), combined Alzheimer's and Parkinson disease and/or MSA, Huntington's disease, synucleinopathies, disorders or conditions characterized by the presence of Lewy bodies, multiple sclerosis, Amyotrophic lateral sclerosis (ALS) dementia (including vascular dementia, Lewy body dementia, Parkinson's dementia, frontotemporal dementia), Down syndrome, Psychosis (including agitation caused by a neurodegenerative disease or associated with dopaminergic therapy such as but not limited to Parkinson's disease psychosis, Alzheimer's disease psychosis, Lewy body dementia psychosis), dyskinesia (including agitation caused by a neurodegenerative disease or associated with dopaminergic therapy), agitation (including agitation caused by a neurodegenerative disease or associated with dopaminergic therapy), conditions associated with dopaminergic therapy (including dystonia, myoclonus, or tremor), synucleinopathies, diseases, disorders or conditions associated with abnormal expression, stability, activities and/or cellular processing of α-synuclein, diseases, disorders or conditions characterized by the presence of Lewy bodies, and combinations thereof.

Embodiments herein are directed to methods of treating a neurodegenerative disease in a subject in need thereof comprising administering to said patient one or more of the compositions described herein. In some embodiments the composition comprises a therapeutically effective amount of 3-phenylsulfonyl-8-piperazinyl-1yl-quinoline or pharmaceutically acceptable salts, hydrates or solvates thereof; a therapeutically effective amount of at least one additional therapeutic agent useful for the treatment of a neurodegenerative disease; and at least one pharmaceutically acceptable excipient; wherein the composition is suitable for oral administration.

In some embodiments, the therapeutically effective amount of 3-phenylsulfonyl-8-piperazinyl-1yl-quinoline or pharmaceutically acceptable salts, hydrates or solvates thereof, and a therapeutically effective amount of at least one additional therapeutic agent useful for treating a neurodegenerative disease, are configured as a single subunit, or two or more subunits.

In some embodiments, the at least one pharmaceutical acceptable excipient is configured into the single subunit, or two or more subunits.

In some embodiments, the single subunit comprises a bar, beads, a block, particles, pellets, granules, fibers, globules, powders, a pill, a capsule, a tablet, a caplet, an orally disintegrating tablet, an osmotic controlled-release oral delivery system and any combination thereof.

In some embodiments, the tablet is a monolayer tablet, a bilayer tablet, or a multilayer tablet or a combination thereof.

In some embodiments, the single subunit further comprises an encapsulation medium.

In some embodiments, wherein the encapsulation medium is a capsule, a soft gel cap, a gel cap, a coating, or any combination thereof.

In some embodiments, the coating comprises a membrane, a film, a wax, a varnish, a glaze, a polymer coating, a sugar coating, a polysaccharide based coating, an enteric coating, or a combination thereof.

In some embodiments, the therapeutically effective amount of 3-phenylsulfonyl-8-piperazinyl-1yl-quinoline or pharmaceutically acceptable salts, hydrates or solvates thereof, and a therapeutically effective amount of at least one additional therapeutic agent useful for treating a neurodegenerative disease are independently configured for immediate release, sustained release, extended release, or any combination thereof.

In some embodiments, the therapeutically effective amount of 3-phenylsulfonyl-8-piperazinyl-1yl-quinoline or pharmaceutically acceptable salts, hydrates or solvates thereof is configured for immediate release, and the additional therapeutic agent useful for treating a neurodegenerative disease is configured for immediate release, sustained release, extended release, or any combination thereof.

In some embodiments, the therapeutically effective amount of 3-phenylsulfonyl-8-piperazinyl-1yl-quinoline or pharmaceutically acceptable salts, hydrates or solvates thereof is configured for sustained release, and the additional therapeutic agent useful for treating a neurodegenerative disease is configured for immediate release, sustained release, extended release, or any combination thereof.

In some embodiments, the therapeutically effective amount of 3-phenylsulfonyl-8-piperazinyl-1yl-quinoline or pharmaceutically acceptable salts, hydrates or solvates thereof is configured for extended release, and the additional therapeutic agent useful for treating a neurodegenerative disease is configured for immediate release, sustained release, extended release, or any combination thereof.

In some embodiments, the two or more subunits independently comprise a bar, beads, a block, particles, pellets, granules, fibers, globules, powders, a pill, a capsule, a tablet, a caplet, an orally disintegrating tablet, an osmotic controlled-release oral delivery system and any combination thereof.

In some embodiments, the tablet is a monolayer tablet, a bilayer tablet, or a multilayer tablet or a combination thereof.

In some embodiments, the two or more subunits comprise a therapeutically effective amount of 3-phenylsulfonyl-8-piperazinyl-1yl-quinoline or pharmaceutically acceptable salts, hydrates or solvates thereof is configured into a first subunit, and the therapeutically effective amount of at least one additional therapeutic agent useful for the treatment of neurodegenerative disease configured into at least one additional subunit.

In some embodiments, the first subunit and the at least one additional subunits are combined into an encapsulation medium.

In some embodiments, wherein the encapsulation medium is a capsule, a soft gel cap, a gel cap, a coating, or any combination thereof.

In some embodiments, the coating comprises a membrane, a film, a wax, a varnish, a glaze, a polymer coating, a sugar coating, a polysaccharide based coating, an enteric coating, or a combination thereof.

In some embodiments, the two or more subunits independently comprise a bar, beads, a block, particles, pellets, granules, fibers, globules, powders, a pill, a capsule, a tablet, a caplet, an orally disintegrating tablet, an osmotic controlled-release oral delivery system and any combination thereof.

In some embodiments, the tablet is a monolayer tablet, a bilayer tablet, or a multilayer tablet or a combination thereof.

In some embodiments, the two or more subunits comprise the therapeutically effective amount of 3-phenylsulfonyl-8-piperazinyl-1yl-quinoline or pharmaceutically acceptable salts, hydrates or solvates thereof configured into a first subunit, and the therapeutically effective amount of at least one additional therapeutic agent useful for the treatment of neurodegenerative disease configured into at least one additional subunit.

In some embodiments, the first subunit and the at least one additional subunits are combined into an encapsulation medium.

In some embodiments, wherein the encapsulation medium is a capsule, a soft gel cap, a gel cap, a coating, or any combination thereof.

In some embodiments, the coating comprises a membrane, a film, a wax, a varnish, a glaze, a polymer coating, a sugar coating, a polysaccharide based coating, an enteric coating, or a combination thereof.

In some embodiments, the therapeutically effective amount of 3-phenylsulfonyl-8-piperazinyl-1yl-quinoline or pharmaceutically acceptable salts, hydrates, polymorphs, or solvates thereof is from about 0.001 mg to about 1,000 mg, about 0.001 mg to about 200 mg, about 0.001 mg to about 175 mg, or 0.001 mg to about 70 mg

In some embodiments, the therapeutically effective amount of 3-phenylsulfonyl-8-piperazinyl-1yl-quinoline or pharmaceutically acceptable salts, hydrates, polymorphs, or solvates thereof is about 15 mg, about 35 mg, or about 70 mg.

In some embodiments, the therapeutically effective amount of 3-phenylsulfonyl-8-piperazinyl-1yl-quinoline or pharmaceutically acceptable salts, hydrates, polymorphs, or solvates thereof is an amount selected from the group consisting of an amount of 3-phenylsulfonyl-8-piperazinyl-1yl-quinoline that may cause convulsions in a subject to which it is administered; an amount that would be expected to exceed the maximum tolerated dose for the subject to which it is administered; an amount associated with systemic exposures characterized by an AUCtau-ss of about 8.2 μg·h/ml, a Cmax of about 0.26 μg/ml; or a combination thereof an mount associated with systemic exposures characterized by an AUC, Cmax, or combinations thereof, that are about 2 to about 3 times higher than the mean clinical exposure achieved at the proposed clinical dose for monotherapy with 3-phenylsulfonyl-8-piperazinyl-1yl-quinoline (i.e. mean AUCtau-ss of about 3.2 μg·h/ml and Cmax of about 0.180 μg/ml), an amount associated with a recorded systemic clinical exposure that is greater than the highest recorded systemic clinical exposure (AUC0-∞ of about 9.25 μg·h/ml and Cmax of about 0.293 μg/ml), an amount of 3-phenylsulfonyl-8-piperazinyl-1yl-quinoline that is greater than about 10 mg/kg/day, an amount of 3-phenylsulfonyl-8-piperazinyl-1yl-quinoline that is greater than 15 mg/day, a dose of 3-phenylsulfonyl-8-piperazinyl-1yl-quinoline that is greater than about 35 mg/day or any combination thereof.

Some embodiments are directed to pharmaceutical compositions comprising a therapeutically effective amount of 3-phenylsulfonyl-8-piperazinyl-1yl-quinoline or pharmaceutically acceptable salts, hydrates, polymorphs, or solvates thereof and at least one pharmaceutically acceptable excipient; wherein the therapeutically effective amount of 3-phenylsulfonyl-8-piperazinyl-1yl-quinoline comprises at least one polymorphic form of 3-phenylsulfonyl-8-piperazinyl-1 yl-quinoline. In some embodiments, the at least one polymorphic form is characterized by a powder x-ray diffraction substantially as shown in any one of FIGS. 1-9. In some embodiments, the at least one polymorphic form is characterized by a powder x-ray diffraction substantially as shown in FIG. 2. In some embodiments, the at least one polymorphic form is characterized by a powder x-ray diffraction substantially as shown in FIG. 3. In some embodiments, the at least one polymorphic form is characterized by a powder x-ray diffraction substantially as shown in FIG. 4. In some embodiments, the at least one polymorphic form is characterized by a powder x-ray diffraction substantially as shown in FIG. 5. In some embodiments, the at least one polymorphic form is characterized by a powder x-ray diffraction substantially as shown in FIG. 6. In some embodiments, the at least one polymorphic form is characterized by a powder x-ray diffraction substantially as shown in FIG. 7. In some embodiments, the at least one polymorphic form is characterized by a powder x-ray diffraction substantially as shown in FIG. 8. In some embodiments, the at least one polymorphic form is characterized by a powder x-ray diffraction substantially as shown in FIG. 9.

In some embodiments, the at least one additional therapeutic agent is selected from the group consisting of an acetylcholinesterase inhibitor, an NMDA receptor antagonist, a 5HT_{2A} inverse agonist or any combination thereof.

In some embodiments, the acetylcholinesterase inhibitor is selected from the group consisting of donepezil, rivastigmine, galantamine, physostigmine, neostigmine, pyridostigmine, ambenonium, demecarium, a phenanthrene derivative, galantamine, caffeine, a piperidine tacrine (also known as tetrahydroaminoacridine), edrophonium, huperzine A, ladostigil, ungeremine, lactucopicrin, 6-[(3-cyclobutyl-2,3,4,5-tetrahydro-1H-3-benzazepin-7-yl)oxy]-N-methyl-3-pyridinecarboxamide hydrochloride or 1-{6-[(3-cyclobutyl-2,3,4,5-tetrahydro-1H-3-benzazepin-7-yl)oxy]-3-pyridinyl}-2-pyrrolidinone or pharmaceutically acceptable salts, hydrates, polymorphs, or solvates thereof.

In some embodiments, the acetylcholinesterase inhibitor is donepezil. In some embodiments, donepezil or pharmaceutically acceptable salts, hydrates, polymorphs, or solvates thereof is donepezil hydrochloride.

In some embodiments, the therapeutically effective amount of donepezil or pharmaceutically acceptable salts, hydrates, polymorphs, or solvates thereof is configured for immediate release, extended release, delayed release, or any combination thereof.

In some embodiments, the therapeutically effective amount of donepezil or pharmaceutically acceptable salts, hydrates, polymorphs, or solvates thereof is from about 0.001 mg to about 1,000 mg, or about 0.001 mg to about 30 mg.

In some embodiments, the therapeutically effective amount of donepezil or pharmaceutically acceptable salts, hydrates, polymorphs, or solvates thereof is about 5 mg, 10 mg, or 23 mg.

In some embodiments, the therapeutically effective amount in an acetylcholinesterase inhibitor is administered to a subject in need thereof in a sub therapeutic amount. In some embodiments, donepezil or pharmaceutically acceptable salts, hydrates or solvates thereof is administered to a subject in need thereof in a daily dose that is considered to sub therapeutic.

In some embodiments, the acetylcholinesterase inhibitor is rivastigmine or pharmaceutically acceptable salts, hydrates, polymorphs, or solvates thereof. In some embodiments, the rivastigmine is rivastigmine tartrate. In some embodiments, the therapeutically effective amount of rivastigmine or pharmaceutically acceptable salts, hydrates, polymorphs, or solvates thereof is configured for immediate release, for extended release, delayed release, or any combination thereof. In some embodiments, the therapeutically effective amount of rivastigmine or pharmaceutically acceptable salts, hydrates, polymorphs, or solvates thereof is from about 0.001 mg to about 1,000 mg, or about 0.001 mg to about 15 mg. In some embodiments, the therapeutically effective amount of rivastigmine or pharmaceutically acceptable salts, hydrates, polymorphs, or solvates thereof is about 1.5 mg, about 3 mg, about 4.5 mg, about 6 mg, about 9 mg, about 9.5 mg, about 12 mg, or about 13.3 mg. In some embodiments, rivastigmine or pharmaceutically acceptable salts, hydrates or solvates thereof is administered to a subject in need thereof in a daily dose that is considered to sub therapeutic.

In some embodiments, the acetylcholinesterase inhibitor is galantamine. In some embodiments, galantamine or pharmaceutically acceptable salts, hydrates, polymorphs, or solvates thereof is galantamine hydrobromide. In some embodiments, the therapeutically effective amount of galantamine or pharmaceutically acceptable salts, hydrates, polymorphs, or solvates thereof is configured for immediate release, extended release, delayed release, or any combination thereof. In some embodiments, the therapeutically effective amount of galantamine or pharmaceutically acceptable salts, hydrates, polymorphs, or solvates thereof is from about 0.001 mg to about 1,000 mg, or about 0.001 mg to about 30 mg. In some embodiments, the therapeutically effective amount of galantamine or pharmaceutically acceptable salts, hydrates, polymorphs, or solvates thereof is about 4 mg, about 8 mg, about 12 mg, about 16 mg, or about 24 mg. In some embodiments, galantamine or pharmaceutically acceptable salts, hydrates or solvates thereof is administered to a subject in need thereof in an amount that is considered to be sub therapeutic.

In some embodiments, NMDA receptor antagonist is selected from the group consisting of memantine, amantadine and ketamine. In some embodiments, the NMDA receptor antagonist is memantine. In some embodiments, the memantine or pharmaceutically acceptable salts, hydrates, polymorphs, or solvates thereof comprises memantine hydrochloride. In some embodiments, the therapeutically effective amount of memantine or pharmaceutically acceptable salts, hydrates, polymorphs, or solvates thereof is configured for extended release, delayed release or any combination thereof. In some embodiments, the therapeutically effective amount of memantine or pharmaceutically acceptable salts, hydrates, polymorphs, or solvates thereof is from about 0.001 mg to about 1,000 mg, or about 0.001 mg to about 30 mg. In some embodiments, the therapeutically effective amount of memantine or pharmaceutically acceptable salts, hydrates, polymorphs, or solvates thereof is about 5 mg, about 7 mg, about 10 mg, about 14 mg, about 20 mg, about 21 mg, or about 28 mg. In some embodiments, memantine or pharmaceutically acceptable salts, hydrates or solvates thereof is administered to a subject in need thereof in an amount that is considered to be sub therapeutic.

In some embodiments, the NMDA receptor antagonist is amantadine. In some embodiments, the amantadine or pharmaceutically acceptable salts, hydrates, polymorphs, or solvates thereof comprises amantadine hydrochloride.

In some embodiments, the therapeutically effective amount of amantadine or pharmaceutically acceptable salts, hydrates, polymorphs, or solvates thereof is configured for immediate release, extended release, delayed release, or any combination thereof.

In some embodiments, the therapeutically effective amount of amantadine or pharmaceutically acceptable salts, hydrates, polymorphs, or solvates thereof is from about 0.001 mg to about 1,000 mg, or about 0.001 mg to about 500 mg. In some embodiments, amantadine or pharmaceutically acceptable salts, hydrates or solvates thereof is administered to a subject in need thereof in an amount that is considered to sub therapeutic.

In some embodiments, the therapeutically effective amount of amantadine or pharmaceutically acceptable salts, hydrates, polymorphs, or solvates thereof is from about 100 mg to about 400 mg.

In some embodiments, the therapeutically effective amount of amantadine or pharmaceutically acceptable salts, hydrates, polymorphs, or solvates thereof is about 100 mg, 200 mg, 300 mg or about 400 mg.

In some embodiments, the 5-HT2A inverse agonist is nelotanserin, pimavanserin, pruvanserin, eplivanserin, volinanserin, glemanserin, ketanserin, ritanserin, clozapine, or pharmaceutically acceptable salts, hydrates, polymorphs, or solvates thereof.

In some embodiments, the nelotanserin or pharmaceutically acceptable salts, hydrates, polymorphs, or solvates thereof comprises Form I of 1-[3-(4-bromo-2-methyl-2H-pyrazol-3-yl)-4-methoxy-phenyl]-3-(2,4-difluoro-phenyl)-urea, Form II of 1-[3-(4-bromo-2-methyl-2H-pyrazol-3-yl)-4-methoxy-phenyl]-3-(2,4-difluoro-phenyl)-urea or a combination thereof. In some embodiments, the 5-HT2A inverse agonist is administered to a subject in need thereof in an amount that is considered to sub therapeutic.

In some embodiments, the therapeutically effective amount of nelotanserin or pharmaceutically acceptable salts, hydrates, polymorphs, or solvates thereof is configured for immediate release, extended release, delayed release, or any combination thereof.

In some embodiments, the therapeutically effective amount of nelotanserin or pharmaceutically acceptable salts, hydrates, polymorphs, or solvates thereof is from about 0.001 mg to about 1,000 mg, or about 0.001 mg to about 100 mg. In some embodiments, nelotanserin or pharmaceutically acceptable salts, hydrates or solvates thereof is administered to a subject in need thereof in an amount that is considered to sub therapeutic.

In some embodiments, the therapeutically effective amount of nelotanserin or pharmaceutically acceptable salts, hydrates, polymorphs, or solvates thereof is about 20 mg, about 40 mg, or about 80 mg.

In some embodiments, the at least one additional therapeutic agent useful for treating a neurodegenerative disease is a lithium compound or pharmaceutically acceptable salts, hydrates, polymorphs or solvates thereof. In some embodiments, the therapeutically effective amount of a lithium compound or pharmaceutically acceptable salts, hydrates, polymorphs, or solvates thereof is configured for extended release, delayed release, or any combination thereof. In some embodiments, the therapeutically effective amount of a lithium compound or pharmaceutically acceptable salts, hydrates, polymorphs or solvates thereof, is from about 0.001 mg to about 1000 mg, from about 0.001 mg to about 500 mg, from about 0.001 mg to about 100 mg, from about 0.001 mg to about 50 mg, from about 0.001 mg to about 10 mg, from about 0.001 mg to about 1 mg, from about 0.001 mg to about 0.1 mg, or from about 0.001 mg to about 0.01 mg. In some embodiments, the therapeutically effective amount of a lithium compound or pharmaceutically acceptable salts, hydrates, polymorphs or solvates thereof, is about 0.01 mg, about 0.1 mg, about 1 mg, about 5 mg, or about 10 mg. In some embodiments, the lithium compound is present in a sub therapeutic amount. In some embodiments, the sub therapeutic amount of a lithium compound or pharmaceutically acceptable salts, hydrates, polymorphs or solvates thereof, is an amount resulting in a serum concentration of between about 0.4 mM and about 1.6 mM, below about 0.4 mM, below about 0.5 mM, below about 0.4 mM, below about 0.3 mM, below about 0.2 mM, below about 0.1 mM, or below about 0.05 mM when administered to a subject. In some embodiments, the therapeutically effective amount of a lithium compound or pharmaceutically acceptable salts, hydrates, polymorphs, or solvates thereof is configured for extended release, delayed release, or any combination thereof.

In some embodiments, the at least one additional therapeutic agent useful for treating a neurodegenerative disease is levodopa or pharmaceutically acceptable salts, hydrates, polymorphs, or solvates thereof. In some embodiments, the therapeutically effective amount of levodopa or pharmaceutically acceptable salts, hydrates, polymorphs, or solvates thereof is configured for immediate release, extended release, delayed release, or any combination thereof. In some embodiments, the therapeutically effective amount of levodopa or pharmaceutically acceptable salts, hydrates, polymorphs, or solvates thereof is from about 0.001 mg to about 10,000 mg, or about 0.001 mg to about 8,000 mg. In some embodiments, the therapeutically effective amount of levodopa or pharmaceutically acceptable salts, hydrates, polymorphs, or solvates thereof is about 285 mg, about 300 mg, about 400 mg, about 435 mg, 500 mg, about 585 mg, about 600 mg, about 700 mg, about 735 mg, about 750 mg, about 800 mg, about 980 mg, about 1,000 mg, about 1,225 mg, about 1,250 mg, about 1,470 mg, about 1,500 mg, about 1,715 mg, about 1,750 mg, about 1,960 mg, about 2,000 mg, about 2,205 mg, about 2,250 mg, about 2,450 mg, about 2,500 mg, about 2,750 mg, about 3,000 mg, about 3,250 mg, about 3,500 mg, about 3,750 mg, about 4,000 mg, about 4,250 mg, about 5,000 mg, about 5,250 mg, about 5,500 mg, about 5,750 mg, about 6,000 mg, about 6,250 mg, about 6,500 mg, about 6,750 mg, about 7,000 mg, about 7,250 mg, about 7,500 mg, about 7,750 mg, or about 8,000 mg. In some embodiments, the at least one additional therapeutic agent useful for treating a neurodegenerative disease is levodopa or pharmaceutically acceptable salts, hydrates, polymorphs, or solvates thereof and carbidopa or pharmaceutically acceptable salts, hydrates, polymorphs, or solvates thereof. In some embodiments, the therapeutically effective amount of levodopa further comprises carbidopa. In some embodiments, the therapeutically effective amount of carbidopa or pharmaceutically acceptable salts, hydrates, polymorphs, or solvates thereof is configured for immediate release, extended release, delayed release, or any combination thereof. In some embodiments, the therapeutically effective amount of carbidopa is from about 0.001 mg to about 1,000 mg, or from about 0.001 mg to about 700 mg. In some embodiments, the therapeutically effective amount of carbidopa is about 30 mg, about 40 mg, about 50 mg, about 60 mg, about 70 mg, about 71.25 mg, about 80 mg, about 108.75 mg, about 146.25 mg, 183.75 mg, about 245 mg, about 245 mg, about 306.25 mg, about 367.5 mg, about 428.75 mg, about 490 mg, about 551.25 mg, or about 612.5 mg.

In some embodiments, the at least one additional therapeutic agent is an anticonvulsant. In some embodiments, anticonvulsants for use herein may include, but are not limited, to levetiracitam (Keppra), AMPA receptor antagonists, barbiturate anticonvulsants, benzodiazepine anticonvulsants, carbamate anticonvulsants, carbonic anhydrase inhibitor anticonvulsants, dibenzazepine anticonvulsants, fatty acid derivative anticonvulsants, gamma-aminobutyric acid analogs, gamma-aminobutyric acid reuptake inhibitors, hydantoin anticonvulsants, miscellaneous anticonvulsants, neuronal potassium channel openers, oxazolidinedione anticonvulsants, pyrrolidine anticonvulsants, succinimide anticonvulsants, triazine anticonvulsants or combinations thereof. In some embodiments, the anticonvulsant is administered to a subject in need thereof in a therapeutically effective amount. In some embodiments, the anticonvulsant or pharmaceutically acceptable salts, hydrates or solvates thereof is administered to a subject in need thereof in an amount that is considered to sub therapeutic.

In some embodiments, the at least one additional therapeutic agent is a monoclonal antibody. In some embodiments, the second therapeutic agent is a human monoclonal antibody. In some embodiments, the second therapeutic agent is a humanized monoclonal antibody. In some embodiments the monoclonal antibody targets beta amyloid. In some embodiments the beta amyloid may comprise aggregated beta amyloid such as but not limited to soluble oligomers, insoluble fibrils deposited into amyloid plaque, or a combination thereof. In some embodiments, the monoclonal antibody is Aducanumab (BIM037), Gantenerumab, Bapineuzumab, Crenezumab, Ponezumab, Solanezumab, SAR228810, MEDI1814, BAN2401, or any combination thereof. In some embodiments, the monoclonal antibody targets alpha-synuclein. In some embodiments, the monoclonal antibody targeting alpha-synuclein is RG-7935, Posiphen, Affitope PD03A, Affitope PD01A, or any combination thereof.

In some embodiments, the at least one additional therapeutic agent is a BACE enzyme inhibitor. In some embodiments, the BACE enzyme inhibitor is CTS-21166, MK-8931, AZD3293, LY3314814, BI 1181181, LY2886721, E2609, RG7129, JNJ-5486911, TAK-070, or any combination thereof.

In some embodiments, the at least one additional therapeutic agent is a RAGE inhibitor. In some embodiments, the RAGE inhibitor is TTP488 (Azeliragon), TTP4000, FPS-ZM1, or any combination thereof.

In some embodiments, the at least one additional therapeutic agent is an antibody targeting Tau. In some embodiments, the antibody targeting Tau is AADVAC-1, AADVAC-2, ACI-35, BMS-986168, RG7345, TRx-237-015 (LMTX), AV-1451, AV-680, Posiphen, or any combination thereof.

In some embodiments, the at least one additional therapeutic agent is a α7 nicotinic acetylcholine receptor modulator. In some embodiments, the α7 nicotinic acetylcholine receptor modulator is Encenicline (EVP-6124), ABT-126, ABT 418, RG3487, Varenicline, A-867744, TC-5219, AVL3288, BMS933043, DSP-3748, or any combination thereof.

In some embodiments, the at least one additional therapeutic agent may include one or more treatments for Alzheimer's disease such as Namzaric™, Exelon®, Aricept® (donepezil hydrochloride), Namenda® (memantine hydrochloride), or galantamine hydrobromide. In some embodiments, described compositions and formulations may be administered in combination with one or more treatments for Parkinson's Disease such as ABT-126 (Abbott Laboratories), pozanicline (Abbott Laboratories), MABT-5102A (AC Immune), Affitope AD-01 (AFFiRiS GmbH), Affitope AD-02 (AFFiRiS GmbH), davunetide (Allon Therapeutics Inc), nilvadipine derivative (Archer Pharmaceuticals), Anapsos (ASAC Pharmaceutical International AIE), ASP-2535 (Astellas Pharma Inc), ASP-2905 (Astellas Pharma Inc), 1 1C-AZD-2184 (AstraZeneca pic), 1 1C-AZD-2995 (AstraZeneca pic), 18F-AZD-4694 (AstraZeneca pic), AV-965 (Avera Pharmaceuticals Inc), AVN-101 (Avineuro Pharmaceuticals Inc), immune globulin intravenous (Baxter International Inc), EVP-6124 (Bayer AG), nimodipine (Bayer AG), BMS-708163 (Bristol-Myers Squibb Co), CERE-110 (Ceregene Inc), CLL-502 (CLL Pharma), CAD-106 (Cytos Biotechnology AG), mimopezil ((Debiopharm SA), DCB-AD1 (Development Centre for Biotechnology), EGb-761 ((Dr Willmar Schwabe GmbH & Co), E-2012 (Eisai Co Ltd), ACC-001 (Elan Corp pic), bapineuzumab (Elan Corp pic), ELND-006(Elan Pharmaceuticals Inc), atomoxetine (Eli Lilly & Co), LY-2811376 (Eli Lilly & Co), LY-451395 (Eli Lilly & Co), m266 (Eli Lilly & Co), semagacestat (Eli Lilly & Co), solanezumab (Eli Lilly & Co), AZD-103 (Ellipsis Neurotherapeutics Inc), FGLL (ENKAM Pharmaceuticals A/S), EHT-0202 (ExonHit Therapeutics SA), celecoxib (GD Searle & Co), GSK-933776A (GlaxoSmithKline pic), rosiglitazone XR (GlaxoSmithKline pic), SB-742457(GlaxoSmithKline pic), R-1578 (Hoffmann-La Roche AG), HF-0220 (Hunter-Fleming Ltd), oxiracetam (ISF Societa Per Azioni), KD-501 (Kwang Dong Pharmaceutical Co Ltd), NGX-267 (Life Science Research Israel), huperzine A (Mayo Foundation), Dimebon (Medivation Inc), MEM-1414 (Memory Pharmaceuticals Corp), MEM-3454 (Memory Pharmaceuticals Corp), MEM-63908 (Memory Pharmaceuticals Corp), MK-0249 (Merck & Co Inc), MK-0752 (Merck & Co Inc), simvastatin (Merck & Co Inc), V-950 (Merck & Co Inc), memantine (Merz & Co GmbH), neramexane (Merz & Co GmbH), Epadel (Mochida Pharmaceutical Co Ltd), 123I-MNI-330 (Molecular Neuroimaging Lie), gantenerumab (MorphoSys AG), NIC5-15 (Mount Sinai School of Medicine), huperzine A (Neuro-Hitech Inc), OXIGON (New York University), NP-12 (Noscira SA), NP-61 (Noscira SA), rivastigmine (Novartis AG), ECT-AD (NsGene A/S), arundic acid (Ono Pharmaceutical Co Ltd), PF-3084014 (Pfizer Inc), PF-3654746 (Pfizer Inc), RQ-00000009 (Pfizer Inc), PYM-50028 (Phytopharm pic), Gero-46(PN Gerolymatos SA), PBT-2 (Prana Biotechnology Ltd), PRX-03140 (Predix Pharmaceuticals Inc), Exebryl-1(ProteoTech Inc), PF-4360365 (Rinat Neuroscience Corp), HuCAL anti-beta amyloid monoclonal antibodies (Roche AG), EVT-302 (Roche Holding AG), nilvadipine (Roskamp Institute), galantamine (Sanochemia Pharmazeutika AG), SAR-110894 (sanofi-aventis), INM-176 (Scigenic & Scigen Harvest), mimopezil (Shanghai Institute of Materia Medica of the Chinese Academy of Sciences), NEBO-178 (Stegram Pharmaceuticals), SUVN-502 (Suven Life Sciences), TAK-065 (Takeda Pharmaceutical), ispronicline (Targacept Inc), rasagiline (Teva Pharmaceutical Industries), T-817MA (Toyama Chemical), PF-4494700 (TransTech Pharma Inc), CX-717 (University of California), 18F-FDDNP (University of California Los Angeles), GTS-21 (University of Florida), 18F-AV-133 (University of Michigan), 18F-AV-45 (University of Michigan), tetrathiomolybdate (University of Michigan), 1231-IMPY (University of Pennsylvania), 18F-AV-1/ZK (University of Pennsylvania), 11C-6-Me-BTA-1 (University of Pittsburgh), 18F-6-OH-BTA-1 (University of Pittsburgh), MCD-386 (University of Toledo), leuprolide acetate implant (Voyager Pharmaceutical Corp), aleplasinin (Wyeth), begacestat (Wyeth), GSI-136 (Wyeth), NSA-789 (Wyeth), SAM-531 (Wyeth), CTS-21166 (Zapaq), and ZSET-1446 (Zenyaku Kogyo).

In some embodiments, the at least one additional therapeutic agent may include one or more agents useful for the treatment of motor neuronal disorders, such as AEOL-10150 (Aeolus Pharmaceuticals Inc), riluzole (Aventis Pharma AG), ALS-08 (Avicena Group Inc), creatine (Avicena Group Inc), arimoclomol (Biorex Research and Development Co), mecobalamin (Eisai Co Ltd), talampanel (Eli Lilly & Co), R-7010 (F Hoffmann-La Roche Ltd), edaravone (Mitsubishi-Tokyo Pharmaceuticals Inc), arundic acid (Ono Pharmaceutical Co Ltd), PYM-50018 (Phytopharm pic), RPI-MN (ReceptoPharm Inc), SB-509 (Sangamo Biosciences Inc), olesoxime (Trophos SA), sodium phenylbutyrate (Ucyclyd Pharma Inc), and R-pramipexole (University of Virginia).

In some embodiments, the compositions described herein may include one or more agents known to modify cholinergic transmission such as M1 muscarinic receptor agonists or allosteric modulators, M2 muscarinic antagonists, acetylcholinesterase inhibitors, nicotinic receptor agonists or allosteric modulators, 5-HT4 receptor partial agonists or 5HT1A receptor antagonists and NMDA receptor antagonists or modulators, glutamate antagonists, GABA-ergic antagonists, H3 antagonists, putative metabolic/mitochondrial modulators, or disease modifying agents such as β or α-secretase inhibitors, Tau-targeted therapeutics, β-amyloid aggregation inhibitors and β-amyloid immunotherapies, an antidepressants, for example a tricyclic, a MAOI (Monoamine oxidase inhibitor) a SSRI (Selective Serotonin Reuptake Inhibitor), a SNRI (Serotonin and Noradrenaline Reuptake Inhibitor) or a NaSSA (noradrenergeric and specific serotonergic antidepressant). Examples of specific antidepressant compounds include amitriptyline, clomipramine, citalopram, dosulepin, doxepin, fluoxetine, imipramine, lofepramine, mirtazapine, moclobemide, nortriptyline, paroxetine, phenelzine, reboxetine, sertraline, tranylcypromine, trazodone, or venlafaxine. In some embodiments, additional therapeutic agents may include antipsychotic drugs, such as olanzapine, clozapine, prisperidone, quentiapine, aripriprazole, or paliperiden.

In some embodiments, the wherein the compositions described herein are administered in the evening, just prior to retiring.

In some embodiments, the compositions described herein are administered orally.

In some embodiments, the compositions described herein are administered to said subject is unchanged or stable during treatment. In some embodiments, the compositions described herein are administered without a dosage titration.

In some embodiments, compositions described herein are administered daily for a period of time, an extended period of time, for the remainder of the subject's life, for an indefinite period of time, for at least one week, for at least one month or for at least 24 weeks.

In some embodiments, the neurodegenerative disease is selected from Alzheimer's disease (including mild or early-stage Alzheimer's disease, mild to moderate Alzheimer's disease, moderate or mid-stage Alzheimer's disease, moderate to severe Alzheimer's disease, moderately severe Alzheimer's disease, severe Alzheimer's disease, Alzheimer's disease with Lewy bodies, (AD)), Parkinson's disease (including Parkinson's disease chemically induced by exposure to environmental agents such as pesticides, insecticides, or herbicides and/or metals such as manganese, aluminum, cadmium, copper, or zinc, SNCA gene-linked Parkinson's disease, sporadic or idiopathic Parkinson's disease, or Parkin- or LRRK2-linked Parkinson's disease (PD)), autosomal-dominant Parkinson's disease, Diffuse Lewy Body Disease (DLBD) also known as Dementia with Lewy Bodies (DLB), Pure Autonomic Failure, Lewy body dysphagia, Incidental LBD, Inherited LBD (e.g., mutations of the alpha-synuclein gene, PARK3 and PARK4), multiple system atrophy (including Olivopontocerebellar Atrophy, Striatonigral Degeneration, Shy-Drager Syndrome (MSA)), combined Alzheimer's and Parkinson disease and/or MSA, Huntington's disease, synucleinopathies, disorders or conditions characterized by the presence of Lewy bodies, multiple sclerosis, Amyotrophic lateral sclerosis (ALS) dementia (including vascular dementia, Lewy body dementia, Parkinson's dementia, frontotemporal dementia), Down syndrome, Psychosis (including agitation caused by a neurodegenerative disease or associated with dopaminergic therapy such as but not limited to Parkinson's disease psychosis, Alzheimer's disease psychosis, Lewy body dementia psychosis), dyskinesia (including agitation caused by a neurodegenerative disease or associated with dopaminergic therapy), agitation (including agitation caused by a neurodegenerative disease or associated with dopaminergic therapy), conditions associated with dopaminergic therapy (including dystonia, myoclonus, or tremor), synucleinopathies, diseases, disorders or conditions associated with abnormal expression, stability, activities and/or cellular processing of α-synuclein, diseases, disorders or conditions characterized by the presence of Lewy bodies, and combinations thereof.

In some embodiments, the neurodegenerative disease is dementia with Lewy Bodies. In some embodiments, the subject is an adult aged 50 to 85, inclusive, with a diagnosis of probable dementia with Lewy Bodies. In some embodiments, the subject with a diagnosis of probable dementia with Lewy Bodies has one of the following: at least two core criteria selected from visual hallucinations, cognitive fluctuations, Parkinsonism, and any combination thereof one core criteria selected from visual hallucinations, cognitive fluctuations, Parkinsonism, and any combination thereof, or at least one suggestive criteria selected from REM sleep behavior disorder, severe neuroleptic sensitivity, low dopamine transporter uptake on a DaT SPECT imaging scan, and any combination thereof.

In some embodiments, the subject is receiving another treatment for dementia with Lewy bodies. In some embodiments, the treatment is selected from stable cholinesterase inhibitor therapy. In some embodiments, the subject is a responder.

In some embodiments, the subject is on stable therapy with donepezil, rivastigmine, galantamine, or any combination thereof. In some embodiments, the stable therapy with donepezil is a stable dose of between about 5 mg and about 23 mg per day. In some embodiments, the stable therapy with rivastigmine is a stable dose between about 3 mg and about 13.3 mg per day. In some embodiments, the stable therapy with galantamine is a stable dose of between about 8 mg to about 24 mg per day.

In some embodiments, treating dementia with Lewy bodies comprises an improvement in the subjects' global function as measured by the Clinician's Interview-Based Impression of Change Plus Caregiver Input (CIBIC-Plus) after 24 weeks of treatment. In some embodiments, treating dementia with Lewy bodies comprises an improvement in the subjects' attention and cognition, as measured by the Choice Reaction Time (CRT) of the CDR computerized cognitive assessment after 24 weeks of treatment. In some embodiments, treating dementia with Lewy bodies comprises an improvement in the subjects' cognition as measured by the Mini Mental Status Examination (MMSE) after 24 weeks of treatment. In some embodiments, treating dementia with Lewy bodies comprises an improvement in the subjects' cognition as measured by the Montreal Cognitive Assessment (MOCA) after 24 weeks of treatment. In some embodiments, treating dementia with Lewy bodies comprises an improvement in the subjects' visual attention and task switching as measured by the Trail Making Test Parts A and B after 24 weeks of treatment. In some embodiments, treating dementia with Lewy bodies comprises an improvement in the subjects' working memory and executive function as measured by the Digit Span Substitution Test (DSST) after 24 weeks of treatment. In some embodiments, treating dementia with Lewy bodies comprises an improvement in the subjects' cognition as measured by the Stroop test after 24 weeks of treatment. In some embodiments, treating dementia with Lewy bodies comprises an improvement in the subjects' cognitive fluctuations and hallucinations as measured by the Neuropsychiatric Inventory 2 (NPI-2) after 24 weeks of treatment. In some embodiments, treating dementia with Lewy bodies comprises an improvement in the subjects' caregiver burden as measured by the Zarit Caregiver Burden Interview (ZBI) after 24 weeks of treatment. In some embodiments, treating dementia with Lewy bodies comprises an improvement in the subjects' sleep quality after 24 weeks of treatment. In some embodiments, treating dementia with Lewy bodies comprises an improvement in the subjects' global function as measured by the Clinician's Interview-Based Impression of Change Plus Caregiver Input (CIBIC-Plus) after 24 weeks of treatment. In some embodiments, treating dementia with Lewy bodies comprises an improvement in the subjects' attention and cognition, as measured by the Choice Reaction Time (CRT) of the CDR computerized cognitive assessment after 12 weeks of treatment. In some embodiments, treating dementia with Lewy bodies comprises an improvement in the subjects' cognition as measured by the Mini Mental Status Examination (MMSE) after 12 weeks of treatment. In some embodiments, treating dementia with Lewy bodies comprises an improvement in the subjects' cognition as measured by the Montreal Cognitive Assessment (MOCA) after 12 weeks of treatment. In some embodiments, treating dementia with Lewy bodies comprises an improvement in the subjects' visual attention and task switching as measured by the Trail Making Test Parts A and B after 12 weeks of treatment. In some embodiments, treating dementia with Lewy bodies comprises an improvement in the subjects' working memory and executive function as measured by the Digit Span Substitution Test (DSST) after 12 weeks of treatment. In some embodiments, treating dementia with Lewy bodies comprises an improvement in the subjects' cognition as measured by the Stroop test after 12 weeks of treatment. In some embodiments, treating dementia with Lewy bodies comprises an improvement in the subjects' cognitive fluctuations and hallucinations as measured by the Neuropsychiatric Inventory 2 (NPI-2) after 12 weeks of treatment. In some embodiments, treating dementia with Lewy bodies comprises an improvement in the subjects' caregiver burden as measured by the Zarit Caregiver Burden Interview (ZBI) after 12 weeks of treatment. In some embodiments, treating dementia with Lewy bodies comprises an improvement in the subjects' sleep quality after 12 weeks of treatment.

In some embodiments, administering one or more of the compositions described herein, for a period of about 24 weeks results in an improvement in cognition, activities of daily living, or a combination thereof.

In some embodiments, the neurodegenerative disease is mild to moderate Alzheimer's disease. In some embodiments, the subject is an adult aged 50 to 85, inclusive, with probable mild to moderate Alzheimer's disease. In some embodiments, the subject is a male or female subject with a clinical diagnosis of probable Alzheimer's disease in accordance the recommendations from the National Institute on Aging-Alzheimer's Association workgroups on diagnostic guidelines for Alzheimer's disease; subject has a documented history of at least 6 months of ongoing donepezil therapy for Alzheimer's disease, with stable dosing of 5 or 10 mg/day for at least the last 2 months and with no intent to change for the duration of the study; subject has an MMSE score 12 to 24 inclusive at Screening and a baseline MMSE score 10 to 26 inclusive; subject has a Hachinski Ischaemia score ≦4 at screening/before administering 3-phenylsulfonyl-8-piperazinyl-1yl-quinoline or pharmaceutically acceptable salts, hydrates, solvates, or polymorphs, thereof; subject has a Magnetic Resonance Imaging (MRI) or computed tomography (CT) scan performed within 12 months before screening/before administering 3-phenylsulfonyl-8-piperazinyl-1yl-quinoline or pharmaceutically acceptable salts, hydrates, solvates, or polymorphs, thereof, with findings consistent with the diagnosis of dementia due to Alzheimer's Disease without any other clinically significant pathologies; subject is an adult aged 50 to 85, inclusive; subject has the ability to comply with procedures for cognitive and other testing; subject lives with (or has substantial periods of contact with) a regular caregiver who is willing to attend all visits, oversee the subject's compliance with treatment procedures and study medication, and report on subject's status, and who has substantial contact with the subject; subject has acceptable general health; or any combination thereof. In some embodiments, if the subject is a female, subject must be of non-childbearing potential or surgically sterile; or, if pre-menopausal or menopausal for 1 year or less, must have a negative pregnancy test and must not be lactating at screening/before administering 3-phenylsulfonyl-8-piperazinyl-1yl-quinoline or pharmaceutically acceptable salts, hydrates, solvates, or polymorphs, thereof. In some embodiments, the subject does not have a diagnosis of possible, probable, or definite vascular dementia in accordance with National Institute of Neurological Disorders and Stroke-Association Internationale pour la Recherche l′Enseignement en Neurosciences (NINDS-AIREN) criteria; a history and/or evidence (CT or MRI scan performed within the past 12 months or at Screening) of any other central nervous system (CNS) disorder that could be interpreted as a cause of dementia (in the opinion of the investigator), e.g., cerebrovascular disease (stroke, hemorrhage); structural or developmental abnormality; epilepsy; infectious, degenerative or inflammatory/demyelinating CNS conditions; or Parkinson's disease; focal findings on the neurological exam (excluding changes attributable to peripheral injury) that are inconsistent with a primary diagnosis of Alzheimer's disease; a history of negative amyloid PET scan or similar brain amyloid imaging, or screen failure from research trial due to negative amyloid imaging within 5 years; atypical clinical features or clinical course of dementia that would lead the investigator to conclude symptoms are more likely due to an alternate dementia diagnosis including, but not limited to, Frontotemporal Dementia, Lewy Body dementia or others; a history of significant psychiatric illness such as schizophrenia or bipolar affective disorder or any other significant psychiatric illness that in the opinion of the investigator would interfere with participation in the study, history of major depressive disorder in the past year, or current active depression requiring treatment, or Geriatric Depression Scale (GDS) >5 at screening/before administering one or more of the compositions described herein; a significant suicide risk as defined by (1) suicidal ideation as endorsed on items 4 or 5 on the Columbia-Suicide Severity Rating Scale (C-SSRS) within the past year, at screening or at baseline; (2) suicidal behaviors within the past year; (3) clinical assessment of significant suicidal risk during patient interview; current psychosis; a history of known or suspected seizures, including febrile seizures (except a single episode of febrile seizure in childhood), unexplained recent loss of consciousness or history of significant head trauma with loss of consciousness; a history of stroke within 2 years of screening/before administering one or more of the compositions described herein; Wernicke's encephalopathy; a known history of photosensitivity or presence of skin conditions (such as porphyria, photo-dermatitis) or treatments (such as medications, ultraviolet light) that may predispose the subject to photosensitivity reactions; a history of malignant melanoma >/=Stage 1 within 5 years; a history or presence of significant cardiovascular, gastrointestinal, endocrine, hepatic, or renal disease or other conditions known to interfere with the absorption, distribution, metabolism, or excretion of drugs, or any other clinically relevant abnormality, medical or psychiatric condition, which, in the opinion of the investigator, makes the subject unsuitable for treatment; a history of myocardial infarction or unstable angina within 1 year of screening or history of more than 1 myocardial infarction within 5 years of screening/before administering one or more of the compositions described herein; a history of alcohol use disorder or other substance abuse, according to the Diagnostic and Statistical Manual of Mental Disorders, 5th Edition (DSM-V) criteria; Unstable or resistant hypertension which in the investigator's opinion makes the patient unsuitable for participation in the trial; postural hypotension (fall in systolic blood pressure of >30 mmHg or fall in diastolic blood pressure of >20 mmHg on standing compared to sitting) at the screening/before administering one or more of the compositions described herein; a QTc ≧450 msec or ≧480 msec for subjects with Bundle Branch Block at the time of screening/before administering one or more of the compositions described herein; alanine transaminase (ALT), aspartate aminotransferase (AST) or alkaline phosphatase values ≧2.5 times upper limit of normal and/or total bilirubin values ≧1.5× the upper limit of normal (ULN) at the time of screening/before administering one or more of the compositions described herein; clinically significant renal laboratory abnormality at the time of screening i.e., serum creatinine ≧1.5×ULN, calculated creatinine clearance <40 mL/min (Cockroft-Gault formula); urinalysis findings such as more than 1+ protein or blood or albumin/creatinine ratio >1.5×ULN (5.1 mg/mmol if upper limit of normal is 3.4 mg/mmol); positive Hepatitis B surface antigen or Hepatitis C antibody test at screening/before administering one or more of the compositions described herein; clinically significant urine drug screen or serum alcohol test; evidence of the following disorders: current vitamin B12 deficiency, positive syphilis serology (unless neurosyphilis was ruled out), or active thyroid dysfunction (particularly suggestive of hypothyroidism), including abnormally high or low serum levels of thyroid stimulating hormone (TSH), where this is thought to be the cause of, or to contribute to the severity of, the subject's dementia. (Note: testing is required for each parameter only when no result is available from the previous 12 months) or any combination thereof. In some embodiments, the subject is a responder.

In some embodiments, the subject is on stable therapy with donepezil. In some embodiments, the subject has been on stable therapy with donepezil for at least 6 months, wherein the stable therapy is a stable dose of 5 mg/day or 10 mg/day for at least 2 months prior to administering one or more of the compositions described herein.

In some embodiments, administering one or more of the compositions described herein, for a period of about 24 weeks results in an improvement in cognition, activities of daily living, or a combination thereof.

In some embodiments, administering one or more of the compositions described herein, for a period of about 24 weeks results in an improvement from baseline in the subjects' Alzheimer's disease Assessment Scale—Cognitive Subscale 11 items (ADAS-Cog-11). In some embodiments, an improvement from baseline in the subjects' Alzheimer's disease Assessment Scale—Cognitive Subscale 11 items (ADAS-Cog-11) is an improvement by at least 3 points after treatment. In some embodiments, administering a therapeutically effective amount of one or more of the compositions described herein, for a period of about 24 weeks results in an improvement from baseline in the subjects' Alzheimer's Disease Cooperative Study—activities of daily living (ADCS-ADL). In some embodiments, an improvement from baseline in the subjects' Alzheimer's Disease Cooperative Study—activities of daily living (ADCS-ADL) is an improvement or no change after treatment. In some embodiments, administering one or more of the compositions described herein, for a period of about 24 weeks results in an improvement baseline in the subjects' ADAS-Cog-13 (ADAS-Cog-11 plus delayed recall and digit cancellation count tests). In some embodiments, an improvement from baseline in the subjects' ADAS-Cog-13 (ADAS-Cog-11 plus delayed recall and digit cancellation count tests) is simultaneously meeting the criteria for ADAS-Cog-11, CIBIC+, and ADCS ADL. In some embodiments, administering a therapeutically effective amount of one or more of the compositions described herein, for a period of about 24 weeks results in an improvement from baseline in the subjects' Global assessment of change will as measured by the Clinician's Interview Based Impression of Change Plus Care Interview (CIBIC+). In some embodiments, an improvement from baseline in the subject's Global assessment of change will as measured by the Clinician's Interview Based Impression of Change Plus Care Interview (CIBIC+) is an improvement or no change after treatment. In some embodiments, administering one or more of the compositions described herein, for a period of about 24 weeks results in an improvement from baseline in the subjects' Neuropsychiatric symptoms and psychopathology as measured by the Neuropsychiatric Inventory (NPI). In some embodiments, administering one or more of the compositions described herein, for a period of about 24 weeks results in an improvement from baseline in the subjects' healthcare resource utilization, caregiver burden, quality of life, or any combination thereof, as measured by the Resource Utilization in Dementia Lite (RUD Lite), the Zarit Caregiver Burden Interview (ZCI), the EuroQol-5D (EQ-5D), the Dependence Scale or any combination thereof.

Embodiments herein are directed to methods of treating a neurodegenerative disease in a subject in need thereof comprising administering different doses of 3-phenylsulfonyl-8-piperazinyl-1yl-quinoline or pharmaceutically acceptable salts, hydrates or solvates thereof, in males and females. In some embodiments, a second therapeutic agent may also be administered in combination with 3-phenylsulfonyl-8-piperazinyl-1yl-quinoline.

Embodiments herein are directed to methods of treating a neurodegenerative disease in a subject in need thereof comprising administering to said patient a therapeutically effective amount of 3-phenylsulfonyl-8-piperazinyl-1yl-quinoline or pharmaceutically acceptable salts, hydrates, polymorphs, or solvates thereof.

In some embodiments, the therapeutically effective amount of 3-phenylsulfonyl-8-piperazinyl-1yl-quinoline or pharmaceutically acceptable salts, hydrates, polymorphs, or solvates thereof is from about 0.001 mg to about 1,000 mg, about 0.001 mg to about 200 mg, about 0.001 mg to about 175 mg, or 0.001 mg to about 70 mg. In some embodiments, the therapeutically effective amount of 3-phenylsulfonyl-8-piperazinyl-1yl-quinoline or pharmaceutically acceptable salts, hydrates, polymorphs, or solvates thereof is about 15 mg, about 35 mg, or about 70 mg. In some embodiments, the therapeutically effective amount of 3-phenylsulfonyl-8-piperazinyl-1yl-quinoline or pharmaceutically acceptable salts, hydrates, polymorphs, or solvates thereof comprises about 70 mg. In some embodiments, the therapeutically effective amount of 3-phenylsulfonyl-8-piperazinyl-1yl-quinoline or pharmaceutically acceptable salts, hydrates, polymorphs, or solvates thereof is administered as a single unit dose of 70 mg per day. In some embodiments, the therapeutically effective amount of 3-phenylsulfonyl-8-piperazinyl-1yl-quinoline or pharmaceutically acceptable salts, hydrates, polymorphs, or solvates thereof is administered as two single unit doses of 35 mg per day. In some embodiments, the two single unit doses of 35 mg per day are administered at the same time. In some embodiments, the two single unit doses of 35 mg per day are administered at different times during the day.

In some embodiments, the therapeutically effective amount of 3-phenylsulfonyl-8-piperazinyl-1yl-quinoline or pharmaceutically acceptable salts, hydrates, polymorphs, or solvates thereof is an amount selected from the group consisting of an amount of 3-phenylsulfonyl-8-piperazinyl-1yl-quinoline that may cause convulsions in a subject to which it is administered; an amount that would be expected to exceed the maximum tolerated dose for the subject to which it is administered; an amount associated with systemic exposures characterized by an AUCtau-ss of about 8.2 μg·h/ml, a Cmax of about 0.26 μg/ml; or a combination thereof an mount associated with systemic exposures characterized by an AUC, Cmax, or combinations thereof, that are about 2 to about 3 times higher than the mean clinical exposure achieved at the proposed clinical dose for monotherapy with 3-phenylsulfonyl-8-piperazinyl-1yl-quinoline (i.e. mean AUCtau-ss of about 3.2 μg·h/ml and Cmax of about 0.180 μg/ml), an amount associated with a recorded systemic clinical exposure that is greater than the highest recorded systemic clinical exposure (AUC0-∞ of about 9.25 μg·h/ml and Cmax of about 0.293 μg/ml), an amount of 3-phenylsulfonyl-8-piperazinyl-1yl-quinoline that is greater than about 10 mg/kg/day, an amount of 3-phenylsulfonyl-8-piperazinyl-1yl-quinoline that is greater than 15 mg/day, a dose of 3-phenylsulfonyl-8-piperazinyl-1yl-quinoline that is greater than about 35 mg/day or any combination thereof.

In some embodiments, the wherein the therapeutically effective amount of 3-phenylsulfonyl-8-piperazinyl-1yl-quinoline or pharmaceutically acceptable salts, hydrates, polymorphs, or solvates thereof is administered in the evening, just prior to retiring.

In some embodiments, the therapeutically effective amount of 3-phenylsulfonyl-8-piperazinyl-1yl-quinoline or pharmaceutically acceptable salts, hydrates, solvates, or polymorphs, thereof, is administered orally.

In some embodiments, the therapeutically effective amount of 3-phenylsulfonyl-8-piperazinyl-1yl-quinoline or pharmaceutically acceptable salts, hydrates, solvates, or polymorphs, thereof, administered to said subject is unchanged or stable during treatment. In some embodiments, the therapeutically effective amount of 3-phenylsulfonyl-8-piperazinyl-1yl-quinoline or pharmaceutically acceptable salts, hydrates, solvates, or polymorphs, thereof is administered without a dosage titration.

In some embodiments, 3-phenylsulfonyl-8-piperazinyl-1yl-quinoline or pharmaceutically acceptable salts, hydrates, solvates, or polymorphs, thereof, is administered daily for a period of time, an extended period of time, for the remainder of the subject's life, for an indefinite period of time, for at least one week, for at least one month or for at least 24 weeks.

In some embodiments, the neurodegenerative disease is selected from Alzheimer's disease (including mild or early-stage Alzheimer's disease, mild to moderate Alzheimer's disease, moderate or mid-stage Alzheimer's disease, moderate to severe Alzheimer's disease, moderately severe Alzheimer's disease, severe Alzheimer's disease, Alzheimer's disease with Lewy bodies, (AD)), Parkinson's disease (including Parkinson's disease chemically induced by exposure to environmental agents such as pesticides, insecticides, or herbicides and/or metals such as manganese, aluminum, cadmium, copper, or zinc, SNCA gene-linked Parkinson's disease, sporadic or idiopathic Parkinson's disease, or Parkin- or LRRK2-linked Parkinson's disease (PD)), autosomal-dominant Parkinson's disease, Diffuse Lewy Body Disease (DLBD) also known as Dementia with Lewy Bodies (DLB), Pure Autonomic Failure, Lewy body dysphagia, Incidental LBD, Inherited LBD (e.g., mutations of the alpha-synuclein gene, PARK3 and PARK4), multiple system atrophy (including Olivopontocerebellar Atrophy, Striatonigral Degeneration, Shy-Drager Syndrome (MSA)), combined Alzheimer's and Parkinson disease and/or MSA, Huntington's disease, synucleinopathies, disorders or conditions characterized by the presence of Lewy bodies, multiple sclerosis, Amyotrophic lateral sclerosis (ALS) dementia (including vascular dementia, Lewy body dementia, Parkinson's dementia, frontotemporal dementia), Down syndrome, Psychosis (including agitation caused by a neurodegenerative disease or associated with dopaminergic therapy such as but not limited to Parkinson's disease psychosis, Alzheimer's disease psychosis, Lewy body dementia psychosis), dyskinesia (including agitation caused by a neurodegenerative disease or associated with dopaminergic therapy), agitation (including agitation caused by a neurodegenerative disease or associated with dopaminergic therapy), conditions associated with dopaminergic therapy (including dystonia, myoclonus, or tremor), synucleinopathies, diseases, disorders or conditions associated with abnormal expression, stability, activities and/or cellular processing of α-synuclein, diseases, disorders or conditions characterized by the presence of Lewy bodies, and combinations thereof.

In some embodiments, the neurodegenerative disease is dementia with Lewy Bodies. In some embodiments, the subject is an adult aged 50 to 85, inclusive, with a diagnosis of probable dementia with Lewy Bodies. In some embodiments, the subject with a diagnosis of probable dementia with Lewy Bodies has one of the following: at least two core criteria selected from visual hallucinations, cognitive fluctuations, Parkinsonism, and any combination thereof one core criteria selected from visual hallucinations, cognitive fluctuations, Parkinsonism, and any combination thereof, or at least one suggestive criteria selected from REM sleep behavior disorder, severe neuroleptic sensitivity, low dopamine transporter uptake on a DaT SPECT imaging scan, and any combination thereof.

In some embodiments, the subject is receiving another treatment for dementia with Lewy bodies. In some embodiments, the treatment is selected from stable cholinesterase inhibitor therapy. In some embodiments, the subject is a responder.

In some embodiments, the subject is on stable therapy with donepezil, rivastigmine, galantamine, or any combination thereof. In some embodiments, the stable therapy with donepezil is a stable dose of between about 5 mg and about 23 mg per day. In some embodiments, the stable therapy with rivastigmine is a stable dose between about 3 mg and about 13.3 mg per day. In some embodiments, the stable therapy with galantamine is a stable dose of between about 8 mg to about 24 mg per day.

In some embodiments, treating dementia with Lewy bodies comprises an improvement in the subjects' global function as measured by the Clinician's Interview-Based Impression of Change Plus Caregiver Input (CIBIC-Plus) after 24 weeks of treatment. In some embodiments, treating dementia with Lewy bodies comprises an improvement in the subjects' attention and cognition, as measured by the Choice Reaction Time (CRT) of the CDR computerized cognitive assessment after 24 weeks of treatment. In some embodiments, treating dementia with Lewy bodies comprises an improvement in the subjects' cognition as measured by the Mini Mental Status Examination (MMSE) after 24 weeks of treatment. In some embodiments, treating dementia with Lewy bodies comprises an improvement in the subjects' cognition as measured by the Montreal Cognitive Assessment (MOCA) after 24 weeks of treatment. In some embodiments, treating dementia with Lewy bodies comprises an improvement in the subjects' visual attention and task switching as measured by the Trail Making Test Parts A and B after 24 weeks of treatment. In some embodiments, treating dementia with Lewy bodies comprises an improvement in the subjects' working memory and executive function as measured by the Digit Span Substitution Test (DSST) after 24 weeks of treatment. In some embodiments, treating dementia with Lewy bodies comprises an improvement in the subjects' cognition as measured by the Stroop test after 24 weeks of treatment. In some embodiments, treating dementia with Lewy bodies comprises an improvement in the subjects' cognitive fluctuations and hallucinations as measured by the Neuropsychiatric Inventory 2 (NPI-2) after 24 weeks of treatment. In some embodiments, treating dementia with Lewy bodies comprises an improvement in the subjects' caregiver burden as measured by the Zarit Caregiver Burden Interview (ZBI) after 24 weeks of treatment. In some embodiments, treating dementia with Lewy bodies comprises an improvement in the subjects' sleep quality after 24 weeks of treatment. In some embodiments, treating dementia with Lewy bodies comprises an improvement in the subjects' global function as measured by the Clinician's Interview-Based Impression of Change Plus Caregiver Input (CIBIC-Plus) after 24 weeks of treatment. In some embodiments, treating dementia with Lewy bodies comprises an improvement in the subjects' attention and cognition, as measured by the Choice Reaction Time (CRT) of the CDR computerized cognitive assessment after 12 weeks of treatment. In some embodiments, treating dementia with Lewy bodies comprises an improvement in the subjects' cognition as measured by the Mini Mental Status Examination (MMSE) after 12 weeks of treatment. In some embodiments, treating dementia with Lewy bodies comprises an improvement in the subjects' cognition as measured by the Montreal Cognitive Assessment (MOCA) after 12 weeks of treatment. In some embodiments, treating dementia with Lewy bodies comprises an improvement in the subjects' visual attention and task switching as measured by the Trail Making Test Parts A and B after 12 weeks of treatment. In some embodiments, treating dementia with Lewy bodies comprises an improvement in the subjects' working memory and executive function as measured by the Digit Span Substitution Test (DSST) after 12 weeks of treatment. In some embodiments, treating dementia with Lewy bodies comprises an improvement in the subjects' cognition as measured by the Stroop test after 12 weeks of treatment. In some embodiments, treating dementia with Lewy bodies comprises an improvement in the subjects' cognitive fluctuations and hallucinations as measured by the Neuropsychiatric Inventory 2 (NPI-2) after 12 weeks of treatment. In some embodiments, treating dementia with Lewy bodies comprises an improvement in the subjects' caregiver burden as measured by the Zarit Caregiver Burden Interview (ZBI) after 12 weeks of treatment. In some embodiments, treating dementia with Lewy bodies comprises an improvement in the subjects' sleep quality after 12 weeks of treatment.

In some embodiments, administering 3-phenylsulfonyl-8-piperazinyl-1yl-quinoline or pharmaceutically acceptable salts, hydrates, solvates, or polymorphs, thereof, for a period of about 24 weeks results in an improvement in cognition, activities of daily living, or a combination thereof.

In some embodiments, the neurodegenerative disease is mild to moderate Alzheimer's disease. In some embodiments, the subject is an adult aged 50 to 85, inclusive, with probable mild to moderate Alzheimer's disease. In some embodiments, the subject is a male or female subject with a clinical diagnosis of probable Alzheimer's disease in accordance the recommendations from the National Institute on Aging-Alzheimer's Association workgroups on diagnostic guidelines for Alzheimer's disease; subject has a documented history of at least 6 months of ongoing donepezil therapy for Alzheimer's disease, with stable dosing of 5 or 10 mg/day for at least the last 2 months and with no intent to change for the duration of the study; subject has an MMSE score 12 to 24 inclusive at Screening and a baseline MMSE score 10 to 26 inclusive; subject has a Hachinski Ischaemia score ≦4 at screening/before administering 3-phenylsulfonyl-8-piperazinyl-1yl-quinoline or pharmaceutically acceptable salts, hydrates, solvates, or polymorphs, thereof; subject has a Magnetic Resonance Imaging (MRI) or computed tomography (CT) scan performed within 12 months before screening/before administering 3-phenylsulfonyl-8-piperazinyl-1yl-quinoline or pharmaceutically acceptable salts, hydrates, solvates, or polymorphs, thereof, with findings consistent with the diagnosis of dementia due to Alzheimer's Disease without any other clinically significant pathologies; subject is an adult aged 50 to 85, inclusive; subject has the ability to comply with procedures for cognitive and other testing; subject lives with (or has substantial periods of contact with) a regular caregiver who is willing to attend all visits, oversee the subject's compliance with treatment procedures and study medication, and report on subject's status, and who has substantial contact with the subject; subject has acceptable general health; or any combination thereof. In some embodiments, if the subject is a female, subject must be of non-childbearing potential or surgically sterile; or, if pre-menopausal or menopausal for 1 year or less, must have a negative pregnancy test and must not be lactating at screening/before administering 3-phenylsulfonyl-8-piperazinyl-1yl-quinoline or pharmaceutically acceptable salts, hydrates, solvates, or polymorphs, thereof. In some embodiments, the subject does not have a diagnosis of possible, probable, or definite vascular dementia in accordance with National Institute of Neurological Disorders and Stroke-Association Internationale pour la Recherche l′Enseignement en Neurosciences (NINDS-AIREN) criteria; a history and/or evidence (CT or MRI scan performed within the past 12 months or at Screening) of any other central nervous system (CNS) disorder that could be interpreted as a cause of dementia (in the opinion of the investigator), e.g., cerebrovascular disease (stroke, hemorrhage); structural or developmental abnormality; epilepsy; infectious, degenerative or inflammatory/demyelinating CNS conditions; or Parkinson's disease; focal findings on the neurological exam (excluding changes attributable to peripheral injury) that are inconsistent with a primary diagnosis of Alzheimer's disease; a history of negative amyloid PET scan or similar brain amyloid imaging, or screen failure from research trial due to negative amyloid imaging within 5 years; atypical clinical features or clinical course of dementia that would lead the investigator to conclude symptoms are more likely due to an alternate dementia diagnosis including, but not limited to, Frontotemporal Dementia, Lewy Body dementia or others; a history of significant psychiatric illness such as schizophrenia or bipolar affective disorder or any other significant psychiatric illness that in the opinion of the investigator would interfere with participation in the study, history of major depressive disorder in the past year, or current active depression requiring treatment, or Geriatric Depression Scale (GDS) >5 at screening/before administering 3-phenylsulfonyl-8-piperazinyl-1yl-quinoline or pharmaceutically acceptable salts, hydrates, solvates, or polymorphs, thereof; a significant suicide risk as defined by (1) suicidal ideation as endorsed on items 4 or 5 on the Columbia-Suicide Severity Rating Scale (C-SSRS) within the past year, at screening or at baseline; (2) suicidal behaviors within the past year; (3) clinical assessment of significant suicidal risk during patient interview; current psychosis; a history of known or suspected seizures, including febrile seizures (except a single episode of febrile seizure in childhood), unexplained recent loss of consciousness or history of significant head trauma with loss of consciousness; a history of stroke within 2 years of screening/before administering 3-phenylsulfonyl-8-piperazinyl-1yl-quinoline or pharmaceutically acceptable salts, hydrates, solvates, or polymorphs, thereof; Wernicke's encephalopathy; a known history of photosensitivity or presence of skin conditions (such as porphyria, photo-dermatitis) or treatments (such as medications, ultraviolet light) that may predispose the subject to photosensitivity reactions; a history of malignant melanoma >/=Stage 1 within 5 years; a history or presence of significant cardiovascular, gastrointestinal, endocrine, hepatic, or renal disease or other conditions known to interfere with the absorption, distribution, metabolism, or excretion of drugs, or any other clinically relevant abnormality, medical or psychiatric condition, which, in the opinion of the investigator, makes the subject unsuitable for treatment; a history of myocardial infarction or unstable angina within 1 year of screening or history of more than 1 myocardial infarction within 5 years of screening/before administering 3-phenylsulfonyl-8-piperazinyl-1yl-quinoline or pharmaceutically acceptable salts, hydrates, solvates, or polymorphs, thereof; a history of alcohol use disorder or other substance abuse, according to the Diagnostic and Statistical Manual of Mental Disorders, 5th Edition (DSM-V) criteria; Unstable or resistant hypertension which in the investigator's opinion makes the patient unsuitable for participation in the trial; postural hypotension (fall in systolic blood pressure of >30 mmHg or fall in diastolic blood pressure of >20 mmHg on standing compared to sitting) at the screening/before administering 3-phenylsulfonyl-8-piperazinyl-1yl-quinoline or pharmaceutically acceptable salts, hydrates, solvates, or polymorphs, thereof a QTc ≧450 msec or ≧480 msec for subjects with Bundle Branch Block at the time of screening/before administering 3-phenylsulfonyl-8-piperazinyl-1yl-quinoline or pharmaceutically acceptable salts, hydrates, solvates, or polymorphs, thereof alanine transaminase (ALT), aspartate aminotransferase (AST) or alkaline phosphatase values ≧2.5 times upper limit of normal and/or total bilirubin values ≧1.5× the upper limit of normal (ULN) at the time of screening/before administering 3-phenylsulfonyl-8-piperazinyl-1yl-quinoline or pharmaceutically acceptable salts, hydrates, solvates, or polymorphs, thereof clinically significant renal laboratory abnormality at the time of screening i.e., serum creatinine ≧1.5×ULN, calculated creatinine clearance <40 mL/min (Cockroft-Gault formula); urinalysis findings such as more than 1+ protein or blood or albumin/creatinine ratio >1.5×ULN (5.1 mg/mmol if upper limit of normal is 3.4 mg/mmol); positive Hepatitis B surface antigen or Hepatitis C antibody test at screening/before administering 3-phenylsulfonyl-8-piperazinyl-1yl-quinoline or pharmaceutically acceptable salts, hydrates, solvates, or polymorphs, thereof; clinically significant urine drug screen or serum alcohol test; evidence of the following disorders: current vitamin B12 deficiency, positive syphilis serology (unless neurosyphilis was ruled out), or active thyroid dysfunction (particularly suggestive of hypothyroidism), including abnormally high or low serum levels of thyroid stimulating hormone (TSH), where this is thought to be the cause of, or to contribute to the severity of, the subject's dementia. (Note: testing is required for each parameter only when no result is available from the previous 12 months) or any combination thereof. In some embodiments, the subject is a responder.

In some embodiments, the subject is on stable therapy with donepezil. In some embodiments, the subject has been on stable therapy with donepezil for at least 6 months, wherein the stable therapy is a stable dose of 5 mg/day or 10 mg/day for at least 2 months prior to administering 3-phenylsulfonyl-8-piperazinyl-1yl-quinoline or pharmaceutically acceptable salts, hydrates, solvates, or polymorphs, thereof.

In some embodiments, administering a therapeutically effective amount 3-phenylsulfonyl-8-piperazinyl-1yl-quinoline or pharmaceutically acceptable salts, hydrates, solvates, or polymorphs, thereof, for a period of about 24 weeks results in an improvement in cognition, activities of daily living, or a combination thereof.

In some embodiments, administering 3-phenylsulfonyl-8-piperazinyl-1yl-quinoline or pharmaceutically acceptable salts, hydrates, solvates, or polymorphs, thereof, for a period of about 24 weeks results in an improvement from baseline in the subjects' Alzheimer ‘s disease Assessment Scale—Cognitive Subscale 11 items (ADAS-Cog-11). In some embodiments, an improvement from baseline in the subjects’ Alzheimer's disease Assessment Scale—Cognitive Subscale 11 items (ADAS-Cog-11) is an improvement by at least 3 points after treatment. In some embodiments, administering a therapeutically effective amount of 3-phenylsulfonyl-8-piperazinyl-1yl-quinoline or pharmaceutically acceptable salts, hydrates, solvates, or polymorphs, thereof, for a period of about 24 weeks results in an improvement from baseline in the subjects' Alzheimer's Disease Cooperative Study—activities of daily living (ADCS-ADL). In some embodiments, an improvement from baseline in the subjects' Alzheimer's Disease Cooperative Study—activities of daily living (ADCS-ADL) is an improvement or no change after treatment. In some embodiments, administering 3-phenylsulfonyl-8-piperazinyl-1yl-quinoline or pharmaceutically acceptable salts, hydrates, solvates, or polymorphs, thereof, for a period of about 24 weeks results in an improvement baseline in the subjects' ADAS-Cog-13 (ADAS-Cog-11 plus delayed recall and digit cancellation count tests). In some embodiments, an improvement from baseline in the subjects' ADAS-Cog-13 (ADAS-Cog-11 plus delayed recall and digit cancellation count tests) is simultaneously meeting the criteria for ADAS-Cog-11, CIBIC+, and ADCS ADL. In some embodiments, administering a therapeutically effective amount of 3-phenylsulfonyl-8-piperazinyl-1yl-quinoline or pharmaceutically acceptable salts, hydrates, solvates, or polymorphs, thereof, for a period of about 24 weeks results in an improvement from baseline in the subjects' Global assessment of change will as measured by the Clinician's Interview Based Impression of Change Plus Care Interview (CIBIC+). In some embodiments, an improvement from baseline in the subject's Global assessment of change will as measured by the Clinician's Interview Based Impression of Change Plus Care Interview (CIBIC+) is an improvement or no change after treatment. In some embodiments, administering 3-phenylsulfonyl-8-piperazinyl-1 yl-quinoline or pharmaceutically acceptable salts, hydrates, solvates, or polymorphs, thereof, for a period of about 24 weeks results in an improvement from baseline in the subjects' Neuropsychiatric symptoms and psychopathology as measured by the Neuropsychiatric Inventory (NPI). In some embodiments, administering 3-phenylsulfonyl-8-piperazinyl-1yl-quinoline or pharmaceutically acceptable salts, hydrates, solvates, or polymorphs, thereof, for a period of about 24 weeks results in an improvement from baseline in the subjects' healthcare resource utilization, caregiver burden, quality of life, or any combination thereof, as measured by the Resource Utilization in Dementia Lite (RUD Lite), the Zarit Caregiver Burden Interview (ZCI), the EuroQol-5D (EQ-5D), the Dependence Scale or any combination thereof.

Some embodiments are directed to the use of the compositions described herein to increase glucose uptake in the brain. Some embodiments are directed to the use of 3-phenylsulfonyl-8-piperazinyl-1yl-quinoline or pharmaceutically acceptable salts, hydrates, polymorphs or solvates thereof to increase glucose uptake in the brain. Some embodiments are directed to the use of the compositions described herein increase glucose utilization in the brain. Some embodiments are directed to the use of 3-phenylsulfonyl-8-piperazinyl-1yl-quinoline or pharmaceutically acceptable salts, hydrates, polymorphs or solvates thereof to increase glucose utilization in the brain.

The present application relates to weight adjusted or body mass index-adjusted dosing of 5-HT₆ receptor antagonists, specifically 3-phenylsulfonyl-8-piperazinyl-1yl-quinoline or pharmaceutically acceptable salts, hydrates, solvates, or polymorphs, thereof for the treatment of a neurodegenerative disease. In some embodiments, the present application relates to methods of dosing 3-phenylsulfonyl-8-piperazinyl-1yl-quinoline or pharmaceutically acceptable salts, hydrates, solvates, or polymorphs, thereof, based on body weight or body mass index (BMI), to treat a neurodegenerative disease. In some embodiments, a second therapeutic agent may also be administered in combination with a 5-HT₆ receptor antagonist or 3-phenylsulfonyl-8-piperazinyl-1yl-quinoline for the treatment of the neurodegenerative disease. In some embodiments, the present application relates to method of dosing a 5-HT₆ receptor antagonist, based on body weight or body mass index, to provide a desired pharmacodynamic response. In some embodiments, the pharmacodynamic response is measured based on receptor occupancy or cognitive studies in patients. In some embodiments, the present application relates to method of dosing 3-phenylsulfonyl-8-piperazinyl-1yl-quinoline or pharmaceutically acceptable salts, hydrates, solvates, or polymorphs, thereof, based on body weight or body mass index, to provide a desired pharmacodynamic response. In some embodiments, the pharmacodynamic response is measured based on receptor occupancy or cognitive studies in patients. In some embodiments, the present application relates to methods of treating a neurodegenerative disease comprising administering different doses of a 5-HT₆ receptor antagonist or pharmaceutically acceptable salts, hydrates or solvates thereof, in males and females. In some embodiments, a second therapeutic agent may also be administered in combination with 5-HT₆ receptor antagonist. In some embodiments, the present application relates to methods of treating a neurodegenerative disease comprising administering different doses of 3-phenylsulfonyl-8-piperazinyl-1yl-quinoline or pharmaceutically acceptable salts, hydrates or solvates thereof, in males and females. In some embodiments, a second therapeutic agent may also be administered in combination with 3-phenylsulfonyl-8-piperazinyl-1yl-quinoline.

The present invention also provides compositions and methods for the differential dosing of a 5-HT₆ receptor antagonist in women and men, based on gender-based differences in their pharmacodynamic effects. The present invention also provides compositions and methods for the differential dosing of 3-phenylsulfonyl-8-piperazinyl-1yl-quinoline in women and men, based on gender-based differences in their pharmacodynamic effects. Such a gender-specific dose may provide an improved method of treating a neurodegenerative disease.

In one embodiment, the present application provides an improved method of administration of a 5-HT₆ receptor antagonist comprising: 1) determining the body mass index (BMI) of a subject; 2) identifying a desired pharmacodynamic response; and 3) administering to the subject a dose of a 5-HT₆ receptor antagonist to achieve a desired pharmacodynamic response based on a comparison of the dose per BMI to pharmacodynamic response. In some embodiments, the pharmacodynamic response may be measured by psychomotor tests or cognitive studies known in the art. In one embodiment, the present application provides an improved method of administration of 3-phenylsulfonyl-8-piperazinyl-1yl-quinoline comprising: 1) determining the body mass index (BMI) of a subject; 2) identifying a desired pharmacodynamic response; and 3) administering to the subject a dose of 3-phenylsulfonyl-8-piperazinyl-1yl-quinoline to achieve a desired pharmacodynamic response based on a comparison of the dose per BMI to pharmacodynamic response. In some embodiments, the pharmacodynamic response may be measured by psychomotor tests or cognitive studies known in the art.

In another embodiment, the present application provides an improved method of administration of a 5-HT₆ receptor antagonist comprising: 1) determining the body weight of a subject; 2) identifying a desired pharmacodynamic response; and 3) administering to the subject a dose of a 5-HT₆ receptor antagonist to achieve a desired pharmacodynamic response based on a comparison of the dose per kilogram of the subject's body weight to pharmacodynamic response. In some embodiments, the pharmacodynamic response may be measured by psychomotor tests or cognitive studies known in the art. In another embodiment, the present application provides an improved method of administration of 3-phenylsulfonyl-8-piperazinyl-1yl-quinoline comprising: 1) determining the body weight of a subject; 2) identifying a desired pharmacodynamic response; and 3) administering to the subject a dose of 3-phenylsulfonyl-8-piperazinyl-1yl-quinoline to achieve a desired pharmacodynamic response based on a comparison of the dose per kilogram of the subject's body weight to pharmacodynamic response. In some embodiments, the pharmacodynamic response may be measured by psychomotor tests or cognitive studies known in the art.

In some embodiments, the pharmacodynamic response may be measured by 5-HT₆ receptor occupancy studies, using radioligands. In some embodiments, the desired pharmacodynamic response may be at least 95% occupancy of 5-HT₆ receptor, at least 90% occupancy of 5-HT₆ receptor, at least 85% occupancy of 5-HT₆ receptor, at least 80% occupancy of 5-HT₆ receptor, at least 70% occupancy of 5-HT₆ receptor, at least 60% occupancy of 5-HT₆ receptor, at least 50% occupancy of 5-HT₆ receptor, at least 40% occupancy of 5-HT₆ receptor, or at least 30% occupancy of 5-HT₆ receptor. The receptor occupancy may be measured in different parts of the central nervous system or the brain, such as putamen, caudate, frontal cortex, and the like.

In some embodiments, the daily dose of 3-phenylsulfonyl-8-piperazinyl-1yl-quinoline used is between 0.1 and 1 mg/BMI/day. In another embodiment, the dosage is between 0.2 and 1 mg/BMI/day, between 0.2 and 2 mg/BMI/day, between 0.2 and 3 mg/BMI/day, between 0.2 and 4 mg/BMI/day, between 0.2 and 5 mg/BMI/day, between 0.2 and 6 mg/BMI/day, or between 0.2 and 10 mg/BMI/day. In some embodiments, the dose of 3-phenylsulfonyl-8-piperazinyl-1yl-quinoline is about 0.1 mg/BMI/day, about 0.2 mg/BMI/day, about 0.5 mg/BMI/day, about 0.75 mg/BMI/day, about 1 mg/BMI/day, about 2 mg/BMI/day, about 3 mg/BMI/day, about 4 mg/BMI/day, about 5 mg/BMI/day, about 6 mg/BMI/day, about 7 mg/BMI/day, about 8 mg/BMI/day, about 9 mg/BMI/day, or about 10 mg/BMI/day. In some embodiments, a second therapeutic agent may also be administered in combination with 3-phenylsulfonyl-8-piperazinyl-1 yl-quinoline. In some embodiments, the daily dose of 3-phenylsulfonyl-8-piperazinyl-1yl-quinoline based on BMI, is lower in females, when compared to males.

In some embodiments, the daily dose of a 5-HT₆ receptor antagonist used is between 0.01 and 0.1 mg/kg body weight/day, between 0.01 and 0.5 mg/kg body weight/day, between 0.01 and 1 mg/kg body weight/day, between 0.01 and 1.5 mg/kg body weight/day, or between 0.01 and 2 mg/kg body weight/day. Specific embodiments include about 0.01 mg/kg body weight/day, about 0.05 mg/kg body weight/day, about 0.1 mg/kg body weight/day, about 0.5 mg/kg body weight/day, about 1 mg/kg body weight/day, or about 2 mg/kg body weight/day. In some embodiments, a second therapeutic agent may also be administered in combination with a 5-HT₆ receptor antagonist. In some embodiments, the daily dose of a 5-HT₆ receptor antagonist based on body weight, is lower in females, when compared to males.

In some embodiments, the daily dose of 3-phenylsulfonyl-8-piperazinyl-1yl-quinoline used is between 0.01 and 0.1 mg/kg body weight/day, between 0.01 and 0.5 mg/kg body weight/day, between 0.01 and 1 mg/kg body weight/day, between 0.01 and 1.5 mg/kg body weight/day, or between 0.01 and 2 mg/kg body weight/day. Specific embodiments include about 0.01 mg/kg body weight/day, about 0.05 mg/kg body weight/day, about 0.1 mg/kg body weight/day, about 0.5 mg/kg body weight/day, about 1 mg/kg body weight/day, or about 2 mg/kg body weight/day. In some embodiments, a second therapeutic agent may also be administered in combination with 3-phenylsulfonyl-8-piperazinyl-1yl-quinoline. In some embodiments, the daily dose of 3-phenylsulfonyl-8-piperazinyl-1yl-quinoline based on body weight, is lower in females, when compared to males.

Accordingly the present invention provides a method for the treatment of a neurodegenerative disease in a patient in need thereof which comprises providing to said patient a dose of a 5-HT₆ receptor antagonist, based on body mass index. In some embodiments, a therapeutically effective amount of an acetylcholinesterase inhibitor, such as, but not limited to donepezil or pharmaceutically acceptable salts, hydrates or solvates thereof, may also be administered. Accordingly the present invention provides a method for the treatment of a neurodegenerative disease in a patient in need thereof which comprises providing to said patient a dose of 3-phenylsulfonyl-8-piperazinyl-1yl-quinoline or pharmaceutically acceptable salts, hydrates, solvates, or polymorphs, thereof, based on body mass index. In some embodiments, a therapeutically effective amount of an acetylcholinesterase inhibitor, such as, but not limited to donepezil or pharmaceutically acceptable salts, hydrates or solvates thereof, may also be administered.

Some embodiments are directed to the use of a combination of a 5-HT₆ receptor antagonist dose based on body weight, and a second therapeutic agent in the manufacture of a medicament for use in the treatment of a neurodegenerative disease. In some embodiments, the second therapeutic agent is an acetylcholinesterase inhibitor, such as, but not limited to donepezil or pharmaceutically acceptable salts, hydrates or solvates thereof. Some embodiments are directed to the use of a combination of a 3-phenylsulfonyl-8-piperazinyl-1yl-quinoline dose based on body weight, and a second therapeutic agent in the manufacture of a medicament for use in the treatment of a neurodegenerative disease. In some embodiments, the second therapeutic agent is an acetylcholinesterase inhibitor, such as, but not limited to donepezil or pharmaceutically acceptable salts, hydrates, polymorphs or solvates thereof.

### EXAMPLES

### Example 1

### Pharmacokinetics and Safety of 3-Phenylsulfonyl-8-Piperazinyl-1yl-Quinoline in Healthy Elderly Adults and Effect of Food in Healthy Adults

To investigate the safety and tolerability of 3-phenylsulfonyl-8-piperazinyl-1yl-quinoline at doses of 35 mg and 70 mg following repeat oral administration in 30 healthy, elderly subjects aged 60-85; to characterize the pharmacokinetics (PK) of 3-phenylsulfonyl-8-piperazinyl-1yl-quinoline at doses of 35 mg and 70 mg following repeat oral administration in healthy, elderly subjects.

Statistical Methods: Safety and PK data will be presented in tabular and/or graphical format and summarized descriptively. To evaluate the effect of food, log-transformed PK parameters will be analyzed by a mixed effect model. The 90 percent confidence interval (CI) for the ratio of population geometric means between the fasted and fed states will be reported for Cmax, AUC(0-∞), AUC(0-t).

Prior to the initiation of a pivotal Phase 3 program with 3-phenylsulfonyl-8-piperazinyl-1yl-quinoline, new tablets for clinical trials must be manufactured using a new manufacturing site. As such, the tablets produced for use in Phase 3 are being evaluated in healthy subjects to demonstrate that the exposure from the new drug product is comparable to that previously described in studies using drug product manufactured by GSK. In addition, the highest dose evaluated in multiple dose studies to date is 50 mg per day. Since 3-phenylsulfonyl-8-piperazinyl-1yl-quinoline is being considered for development in other Central Nervous System (CNS) disorders in older adults, an evaluation of the PK and safety at a higher dose is warranted to enable higher doses in future studies for other indications. The effect of food on 3-phenylsulfonyl-8-piperazinyl-1 yl-quinoline pharmacokinetics was established early in the development program at a 50 mg dose and with a capsule formulation.

The 35 mg dose was evaluated in four Phase 2 trials and is the dose being evaluated in a Phase 3 pivotal study. In the AZ310866 Phase 2b study, there was a dose dependent increase in efficacy vs placebo in the ADAS-Cog score between 15 mg (−0.7 units) and 35 mg (−1.7 units). These data suggest that further benefit may be achieved with doses higher than 35 mg as higher plasma concentrations could produce an incremental increase in efficacy. These benefits need to be balanced with the potential for adverse events, in particular, the CNS toxicity observed in dogs and rabbits described below. In nonclinical studies, 3-phenylsulfonyl-8-piperazinyl-1yl-quinoline caused seizures in rabbits and dogs but not in rodents (mice or rats). In the rat maximal electroshock seizure threshold test, 3-phenylsulfonyl-8-piperazinyl-1yl-quinoline did not decrease the seizure threshold at an extrapolated Cmax of 1887 ng/mL. In rabbits, seizures were produced after a single dose at 300 mg/kg, which exceeded the maximum tolerated repeat-dose level (MTD). In dogs, seizures occurred in 2 dogs only after daily dosing for 8 weeks at the MTD (3 weeks at 10 mg/kg/day followed by 5 weeks at 15 mg/kg/day), but did not occur when the dose level was reduced for the rest of the 26-week study or in dogs given 7.5 mg/kg/day for the entire 26 weeks. In the 26-week dog study, one high-dose dog had seizures on Day 55 and was euthanized. A second dog had seizures on Day 59 and survived. For the second dog, plasma samples taken approximately 5 minutes and two hours after the seizure (4 and 6 hours post dose on Day 59) had SB742457 concentrations of 1570 and 1440 ng/mL, respectively. For the first dog that experienced a seizure on Day 55, there are no plasma concentration data at the time of seizure; however, this dog had a Cmax of 1700 ng/mL on Day 53/54. In summary, a plasma concentration >1570 ng/mL may be associated with an increased seizure risk in dogs (of note, other mid- and high-dose dogs that did not experience any seizure activity achieved plasma concentrations of up to 1937 ng/mL). In study SB742457/005, elderly subjects received 35 mg once daily of 3-phenylsulfonyl-8-piperazinyl-1yl-quinoline for 28 days. The mean Cmax in this study was 181 ng/ml in males and 177 ng/ml in females. The highest recorded Cmax in this study was 307 ng/ml. Given the linear human pharmacokinetics established in the phase I and II clinical trials, multiple dosing with a 70 mg 3-phenylsulfonyl-8-piperazinyl-1yl-quinoline dose would be expected to produce a mean Cmax value of approximately 360 ng/mL and a maximum value of 714 ng/ml in patients. This mean value is approximately ¼th the Cmax value observed in dogs with seizures. The maximum concentration that may be achieved is approximately ½ the Cmax value observed in the 2 dogs with seizures. To further understand the risk to humans, SimCYP population PBPK modelling was used to predict brain concentrations of 3-phenylsulfonyl-8-piperazinyl-1yl-quinoline in dogs exposed to the concentrations linked with seizures, and to compare these with predicted human brain concentrations at the clinical dose of 35 mg. The simulations predicted that the human steady-state brain concentrations following repeat administration with 35 mg would be approximately 40-fold lower than the brain concentrations associated with seizures in dogs. Assuming linear pharmacokinetics, the human steady-state brain concentrations with 70 mg would be approximately 20-fold lower than the brain concentrations associated with convulsions in dogs. Upon review of clinical data, no seizures were observed in studies with healthy subjects (n=225) who received single doses of up to 175 mg and repeat doses of up to 50 mg for 13 days. Furthermore, in Phase 2 studies encompassing 1024 patients with Alzheimer's disease at doses of 5 mg to 35 mg per day, two subjects reported seizures, both in the Phase 2b study with 3-phenylsulfonyl-8-piperazinyl-1yl-quinolone administered as adjunctive therapy to donepezil. One subject was in the placebo group and one in the 15 mg 3-phenylsulfonyl-8-piperazinyl-1yl-quinoline group. The subject receiving 3-phenylsulfonyl-8-piperazinyl-1yl-quinoline was hospitalized with a suspicion of a TIA and experienced a seizure, which was reported by the PI as not attributable to study drug. Overall, these data suggest efficacy without seizure at doses higher than 30 mg, contrary to that predicted by the animal models.

Part 1 is a placebo-controlled, randomized, repeat dose study of 3-phenylsulfonyl-8-piperazinyl-1yl-quinoline in two cohorts of healthy, elderly subjects. Subjects will be admitted to the clinical unit on Day-1 and remain in the unit until Day 8. Each subject will receive single 35 mg or 70 mg doses of 3-phenylsulfonyl-8-piperazinyl-1yl-quinoline/placebo for 7 days. The 70 mg cohort will be dosed in groups of three and separated by at least 3 days. Safety assessments will be collected throughout the treatment period. Serial PK samples will be collected throughout the treatment period and for up to 168 hours following the last dose of study drug (via outpatient visits). Each subject will participate in the study for approximately 7 weeks i.e., 30 day screening period, 1-week treatment period, and a 10-14 day follow-up period.

All laboratory tests with values that are considered clinically significantly abnormal during participation in the study should be repeated until the values return to normal or baseline. If such values do not return to normal within a period judged reasonable by the investigator, the etiology should be identified and the sponsor notified.

Blood samples for PK analysis of 3-phenylsulfonyl-8-piperazinyl-1yl-quinoline and metabolites will be collected at the time points indicated in Time and Events Tables. The actual date and time of each blood sample collection will be recorded. The timing of PK samples may be altered and/or PK samples may be obtained at additional time points to ensure thorough PK monitoring.

Final analysis will be performed after the completion of the study and final datasets authorization. Data listings will be sorted by subject, period, day/time, and treatment; summaries will be presented by treatment, day/time. Subjects received placebo in Cohorts 1 and 2 will be combined. Unless stated otherwise, descriptive summaries will include n, mean, standard deviation (SD), coefficient of variation (% CV), median, minimum, and maximum for continuous variables, n and percent for categorical variables, and geometric mean, 95% confidence interval (CI), and the between-subject CV (% CVb) based on the geometric mean for the loge-transformed PK parameters. Version 9.2 or higher of the SAS system will be used to analyze the data as well as to generate tables, figures, and listings. Complete details will be documented in the Statistical Analysis Plan (SAP).

Plasma 3-phenylsulfonyl-8-piperazinyl-1yl-quinoline concentration-time data will be analyzed by non-compartmental methods with Phoenix WinNonlin or other pharmacokinetic software programs. Calculations will be based on the actual sampling times recorded during the study. From the plasma concentration-time data, the primary pharmacokinetic parameters will be determined for: Part 1: AUC(0-τ), Cτ, Cmin, Cmax, CL/F, tmax, and t1/2.

Additional PK parameters may be calculated. Pharmacokinetic data will be presented in graphical and tabular form and will be summarized descriptively. The planed statistical comparisons for PK parameters are listed below.

The dose proportionality between the 2 doses will be assessed using an ANOVA model based on the dose-normalized PK parameter. The parameters will be loge transformed prior to analysis. The ratio of geometric least squares (GLS) means and the corresponding 90% confidence interval will be estimated for AUC(0-τ), Cτ and Cmin, Cmax.

Additional comparisons may be performed and details on PK analyses will be provided in the SAP.

### Example 2

### 70 mg 3-phenylsulfonyl-8-piperazinyl-1yl-quinoline

A tablet containing 70 mg of 3-phenylsulfonyl-8-piperazinyl-1yl-quinoline as the active ingredient was prepared according to the following:

| | Theoretical Unit | | |
|---|---|---|---|
| | Quantity | | Reference to |
| Component | (mg/tablet) | Function | Standard |
| Tablet Strength | 70 | NA | NA |
| Intra-granular | | | |
| 3-phenylsulfonyl-8-piperazinyl-1yl- | 70.0 | Active | In House |
| quinoline | | | |
| Microcrystalline cellulose | 25-30 | Filler | Ph Eur. and USP/NF |
| Mannitol | 33.8-27.8 | Filler | Ph Eur. and USP/NF |
| Sodium starch glycolate | 4.2 | Disintegrant | Ph. Eur. and USP/NF |
| Hypromellose 2910 | 7.0 | Binder | Ph. Eur. and USP/NF |
| Purified water | qs | Binding Fluid | Ph. Eur. and USP/NF |
| Extra-granular | | | |
| Mannitol | 89.5-83.5 | Filler | Ph Eur. and USP/NF |
| Microcrystalline cellulose | 57-63 | Filler | Ph Eur. and USP/NF |
| Sodium starch glycolate | 10.5 | Disintegrant | Ph Eur. and USP/NF |
| Magnesium stearate | 3 | Lubricant | Ph Eur. and USP/NF |
| Tablet Core Weight | 300.0 | — | — |

### Example 3

### 35 mg 3-phenylsulfonyl-8-piperazinyl-1yl-quinoline/5 mg donepezil

A tablet containing 35 mg of 3-phenylsulfonyl-8-piperazinyl-1yl-quinoline/5 mg donepezil as the active ingredients was prepared according to the following:

| | Unit weight | Unit weight | Unit weight |
|---|---|---|---|
| Component Name | mg/tab | mg/tab | mg/tab |
| 3-phenylsulfonyl-8-piperazinyl-1yl-quinoline | 35 | 35 | 35 |
| donepezil HCl | 5 | 5 | 5 |
| Microcrystalline Cellulose NF (Avicel PH101) | 60-50 | | |
| Mannitol USP (Pearlitol 160C, USP) | 25-35 | | |
| Sodium Starch Glycolate, NF (Intragranular) | 4.5 | | |
| Hydroxypropyl Methylcellulose 2910 USP 6 | 9 | | |
| CPS | | | |
| Microcrystalline Cellulose NF, Ph. Eur, JP | 64-52 | 145-140 | 155-150 |
| (Avicel PH102) | | | |
| Mannitol Pearlitol 200SD Roquette | 36-44 | 50-55 | 40-45 |
| Sodium Starch Glycolate, NF | 8.5-12.5 | 12.5 | |
| Magnesium Stearate NF/EP Non-Bovine | 3 | 2.5 | 2.5 |
| #5712 | | | |
| Croscarmellose sodium | | | 12.5 |
| Tablet Weight | 250 | 250 | 250 |

### Example 4

### 35 mg 3-phenylsulfonyl-8-piperazinyl-1yl-quinoline/10 mg donepezil

A tablet containing 35 mg of 3-phenylsulfonyl-8-piperazinyl-1yl-quinoline/10 mg donepezil as the active ingredients was prepared according to the following:

| | Unit weight | Unit weight | Unit weight |
|---|---|---|---|
| Component Name | mg/tab | mg/tab | mg/tab |
| 3-phenylsulfonyl-8-piperazinyl-1yl-quinoline | 35 | 35 | 35 |
| donepezil HCl | 10 | 10 | 10 |
| Microcrystalline Cellulose NF (Avicel PH101) | 45-55 | | |
| Mannitol USP (Pearlitol 160C, USP) | 35-25 | | |
| Sodium Starch Glycolate, NF (Intragranular) | 4.5 | | |
| Hydroxypropyl Methylcellulose 2910 USP 6 | 9 | | |
| CPS | | | |
| Microcrystalline Cellulose NF, Ph. Eur, JP | 55-50 | 135-145 | 145-155 |
| (Avicel PH102) | | | |
| Mannitol Pearlitol 200SD Roquette | 42-47 | 45-44 | 35-45 |
| Sodium Starch Glycolate, NF | 9.5-12.5 | 12.5 | |
| Magnesium Stearate NF/EP Non-Bovine | 3 | 2.5 | 2.5 |
| #5712 | | | |
| Croscarmellose sodium | | | 12.5 |
| Tablet Weight | 250 | 250 | 250 |

### Example 5

### Bilayer tablet 35 mg 3-phenylsulfonyl-8-piperazinyl-1yl-quinoline/5 mg donepezil

A bilayer tablet containing 35 mg of 3-phenylsulfonyl-8-piperazinyl-1yl-quinoline/5 mg donepezil as the active ingredients was prepared according to the following:

| | Unit weight |
|---|---|
| Component Name | mg/tab |
| 3-phenylsulfonyl-8-piperazinyl-1yl-quinoline | 35 |
| Microcrystalline Cellulose NF (Avicel PH101) | 12.5-15.5 |
| Mannitol USP (Pearlitol 160C, USP) | 16-14 |
| Sodium Starch Glycolate, NF | 7.5-6.5 |
| Hydroxypropyl Methylcellulose 2910 USP 6 CPS | 3-4 |
| Microcrystalline Cellulose NF, Ph. Eur, JP (Avicel | 30-36 |
| PH102) | |
| Mannitol Pearlitol 200SD Roquette | 43-37 |
| Magnesium Stearate NF/EP Non-Bovine #5712 | 1.5-2.5 |
| Total layer weight | 150 |
| Donepezil HCl | 5 |
| Sodium Starch Glycolate, NF | 7-8 |
| Hydroxypropyl Methylcellulose 2910 USP 6 CPS | 9-5 |
| Microcrystalline Cellulose NF, Ph. Eur, JP (Avicel | 62-59 |
| PH102) | |
| Mannitol Pearlitol 200SD Roquette | 16-21 |
| Magnesium Stearate NF/EP Non-Bovine #5712 | 1-2 |
| Total layer weight | 100 |
| Total tablet weight | 250 |

### Example 6

### Bilayer tablet 35 mg 3-phenylsulfonyl-8-piperazinyl-1yl-quinoline/10 mg donepezil

A bilayer tablet containing 35 mg of 3-phenylsulfonyl-8-piperazinyl-1yl-quinoline/10 mg donepezil as the active ingredients was prepared according to the following:

| | Unit weight |
|---|---|
| Component Name | mg/tab |
| 3-phenylsulfonyl-8-piperazinyl-1yl-quinoline | 35 |
| Microcrystalline Cellulose NF (Avicel PH101) | 16-12 |
| Mannitol USP (Pearlitol 160C, USP) | 14-16.5 |
| Sodium Starch Glycolate, NF | 8-6 |
| Hydroxypropyl Methylcellulose 2910 USP 6 | 3-4 |
| CPS | |
| Microcrystalline Cellulose NF, Ph. Eur, JP | 32-28 |
| (Avicel PH102) | |
| Mannitol Pearlitol 200SD Roquette | 40-47 |
| Magnesium Stearate NF/EP Non-Bovine | 1.5-2 |
| #5712 | |
| Total layer weight | 150 |
| Donepezil HCl | 10 |
| Sodium Starch Glycolate, NF | 7-8 |
| Hydroxypropyl Methylcellulose 2910 USP 6 | 6-4 |
| CPS | |
| Microcrystalline Cellulose NF, Ph. Eur, JP | 61-55 |
| (Avicel PH102) | |
| Mannitol Pearlitol 200SD Roquette | 15-21 |
| Magnesium Stearate NF/EP Non-Bovine | 1-2 |
| #5712 | |
| Total layer weight | 100 |
| Total tablet weight | 250 |

### Example 7

### In Vivo Alterations in Brain Glucose Utilization with RVT-101, a 5HT6 Inhibitor for the Treatment of Alzheimer's Disease

3-phenylsulfonyl-8-piperazinyl-1yl-quinoline is a potent antagonist of the 5-hydroxytryptamine 6 (5-HT6) serotonin receptor. Reduced glucose utilization is an early sign of decreasing brain function in AD. Altered neuronal glucose uptake assessed by flurodeoxyglucose-positron emission tomography (FDG-PET) may be useful as a diagnostic foundation for identifying patients with AD. This study evaluates 3-phenylsulfonyl-8-piperazinyl-1yl-quinoline at clinically relevant doses on healthy rat brain glucose utilization using FDG-PET/CT.

Methods: Healthy, young CD rats were pre-dosed with vehicle (1% methylcellulose, w/v) or RVT-101 (10 mg/kg or 20 mg/kg) 3 h prior to FDG (18.1±3.5 MBq, i.v.). Brain FDG standard uptake values (SUV) were quantified using scanning in single 20 min list mode (BioPET/CT, Sedecal) starting 30 min post FDG dosing. PET/CT scans were analyzed as a single time frame summarizing counts over 20 min. All experiments were completed in an accredited facility according to NIH guidelines. PET/CT imaging data were analyzed using Student's t-test, cluster analysis, and multivariate analysis of variance (MANOVA) using a subset of SUV from 5 cluster-identified regions.

Results: 3-phenylsulfonyl-8-piperazinyl-1yl-quinoline increased glucose uptake in healthy young CD rats in a dose dependent manner in almost all brain regions, but pairwise comparisons did not reach statistical significance.). SUV from 5 regions that clustered separately were identified: acumbens, cerebellum (white), orbitofrontal cortex, insular cortex and pituitary. Analyzing the composite SUV profile from 5 brain regions in a linear regression model showed a statistically significant overall dose effect (p=0.0006) Further, cluster analysis revealed a placebo outlier, which when removed, provide additional support of a drug effect on glucose uptake in sub-regions.

Conclusion: Glucose uptake after pre-treatment with 3-phenylsulfonyl-8-piperazinyl-1yl-quinoline compared to vehicle pre-treatment showed a trend of increased glucose uptake in the majority of brain regions analyzed, especially in the 20 mg/kg group. Selection of a subset of regions for regression analysis revealed a statistically significant dose effect related to glucose uptake. These data suggest that 3-phenylsulfonyl-8-piperazinyl-1yl-quinoline may increase brain glucose utilization, an important biologic marker for drugs being developed for Alzheimer's Disease.

Although the present disclosure has been described in considerable detail with reference to certain preferred versions thereof, other versions are possible. Therefore, the spirit and scope of the application should not be limited to the description of the preferred versions described herein.

Although compositions, materials, and methods similar or equivalent to those described herein can be used in the practice or testing of the present invention, suitable preparations, methods and materials are described herein. All publications mentioned herein are incorporated by reference in their entirety. In the case of conflict, the present specification, including definitions will control. In addition, the particular embodiments discussed below are illustrative only and not intended to be limiting.

All features disclosed in the specification, including the abstract and drawings, and all the steps in any method or process disclosed, may be combined in any combination, except combinations where at least some of such features and/or steps are mutually exclusive. Each feature disclosed in the specification, including abstract and drawings, can be replaced by alternative features serving the same, equivalent or similar purpose, unless expressly stated otherwise. Thus, unless expressly stated otherwise, each feature disclosed is one example only of a generic series of equivalent or similar features. Various modifications of the application, in addition to those described herein, will be apparent to those skilled in the art from the foregoing description. Such modifications are also intended to fall within the scope of the appended claims.

Unless otherwise indicated, all numbers expressing quantities of ingredients, properties such as molecular weight, reaction conditions, and so forth used in the specification and claims are to be understood as being modified in all instances by the term “about.” As used herein, the term “about” means plus or minus 10% of a given value. For example, “about 50%” means in the range of 45%-55%. Accordingly, unless indicated to the contrary, the numerical parameters set forth in the specification and attached claims are approximations that may vary depending upon the desired properties sought to be obtained by the present invention. At the very least, and not as an attempt to limit the application of the doctrine of equivalents to the scope of the claims, each numerical parameter should at least be construed in light of the number of reported significant digits and by applying ordinary rounding techniques. Notwithstanding that the numerical ranges and parameters setting forth the broad scope of the invention are approximations, the numerical values set forth in the specific examples are reported as precisely as possible. Any numerical value, however, inherently contains certain errors necessarily resulting from the standard deviation found in their respective testing measurements.

Recitation of ranges of values herein is merely intended to serve as a shorthand method of referring individually to each separate value falling within the range. Unless otherwise indicated herein, each individual value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein is intended merely to better illuminate the invention and does not pose a limitation on the scope of the invention otherwise claimed. No language in the specification should be construed as indicating any non-claimed element essential to the practice of the invention.

Groupings of alternative elements or embodiments of the invention disclosed herein are not to be construed as limitations. Each group member may be referred to and claimed individually or in any combination with other members of the group or other elements found herein. It is anticipated that one or more members of a group may be included in, or deleted from, a group for reasons of convenience and/or patentability. When any such inclusion or deletion occurs, the specification is deemed to contain the group as modified thus fulfilling the written description of all Markush groups used in the appended claims.

Certain embodiments of this invention are described herein, including the best mode known to the inventors for carrying out the invention. Of course, variations on these described embodiments will become apparent to those of ordinary skill in the art upon reading the foregoing description. The inventor expects skilled artisans to employ such variations as appropriate, and the inventors intend for the invention to be practiced otherwise than specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

Specific embodiments disclosed herein may be further limited in the claims using “consisting of” or “consisting essentially of” language, rather than “comprising”. When used in the claims, whether as filed or added per amendment, the transition term “consisting of” excludes any element, step, or ingredient not specified in the claims. The transition term “consisting essentially of” limits the scope of a claim to the specified materials or steps and those that do not materially affect the basic and novel characteristic(s). Embodiments of the invention so claimed are inherently or expressly described and enabled herein.

In closing, it is to be understood that the embodiments of the invention disclosed herein are illustrative of the principles of the present invention. Other modifications that may be employed are within the scope of the invention. Thus, by way of example, but not of limitation, alternative configurations of the present invention may be utilized in accordance with the teachings herein. Accordingly, the present invention is not limited to that precisely as shown and described.

What is claimed is:

1. A composition comprising:
a therapeutically effective amount of 3-phenylsulfonyl-8-piperazinyl-1yl-quinoline or pharmaceutically acceptable salts, hydrates or solvates thereof;
a therapeutically effective amount of at least one additional therapeutic agent useful for the treatment of neurodegenerative disease; and
at least one pharmaceutically acceptable excipient;
wherein the composition is suitable for oral administration.

2. The composition of claim 1, wherein the therapeutically effective amount of 3-phenylsulfonyl-8-piperazinyl-1yl-quinoline or pharmaceutically acceptable salts, hydrates or solvates thereof, and a therapeutically effective amount of at least one additional therapeutic agent useful for treating a neurodegenerative disease are configured as a single subunit or as two or more subunits.

3. The composition of claim 2, wherein the at least one pharmaceutical acceptable excipient is configured into the single subunit or the two or more subunits.

4. The composition of claim 2, wherein the single subunit or the two or more subunits is independently selected from a bar, beads, a block, particles, pellets, granules, fibers, globules, powders, a pill, a capsule, a tablet, a caplet, an orally disintegrating tablet, an osmotic controlled-release oral delivery system and any combination thereof.

5. The composition of claim 4, wherein the tablet is a monolayer tablet, a bilayer tablet, or a multilayer tablet, or a combination thereof.

6. The composition of claim 2, wherein the composition further comprises an encapsulation medium.

7. The composition of claim 6, wherein the encapsulation medium is a capsule, a soft gel cap, a gel cap, a coating, or any combination thereof.

8. The method of claim 7, wherein the coating comprises a membrane, a film, a wax, a varnish, a glaze, a polymer coating, a sugar coating, a polysaccharide based coating, an enteric coating, or a combination thereof.

9. The composition of claim 1, wherein the therapeutically effective amount of 3-phenylsulfonyl-8-piperazinyl-1yl-quinoline or pharmaceutically acceptable salts, hydrates or solvates thereof, and a therapeutically effective amount of at least one additional therapeutic agent useful for treating a neurodegenerative disease are independently configured for immediate release, sustained release, extended release, or any combination thereof.

10. The composition of claim 9, wherein the therapeutically effective amount of 3-phenylsulfonyl-8-piperazinyl-1yl-quinoline or pharmaceutically acceptable salts, hydrates or solvates thereof is configured for immediate release, and the additional therapeutic agent useful for treating a neurodegenerative disease is configured for immediate release, sustained release, extended release, or any combination thereof.

11. The composition of claim 9, wherein the therapeutically effective amount of 3-phenylsulfonyl-8-piperazinyl-1yl-quinoline or pharmaceutically acceptable salts, hydrates or solvates thereof is configured for sustained release, and the additional therapeutic agent useful for treating a neurodegenerative disease is configured for immediate release, sustained release, extended release, or any combination thereof.

12. The composition of claim 9, wherein the therapeutically effective amount of 3-phenylsulfonyl-8-piperazinyl-1yl-quinoline or pharmaceutically acceptable salts, hydrates or solvates thereof is configured for extended release, and the additional therapeutic agent useful for treating a neurodegenerative disease is configured for immediate release, sustained release, extended release, or any combination thereof.

13. The composition of claim 2, wherein the two or more subunits independently comprise a bar, beads, a block, particles, pellets, granules, fibers, globules, powders, a pill, a capsule, a tablet, a caplet, an orally disintegrating tablet, an osmotic controlled-release oral delivery system and any combination thereof.

14. The composition of claim 13, wherein the tablet is a monolayer tablet, a bilayer tablet, or a multilayer tablet, or a combination thereof.

15. The composition of claim 2, wherein the two or more subunits comprise a therapeutically effective amount of 3-phenylsulfonyl-8-piperazinyl-1yl-quinoline or pharmaceutically acceptable salts, hydrates or solvates thereof is configured into a first subunit, and the therapeutically effective amount of at least one additional therapeutic agent useful for the treatment of neurodegenerative disease configured into at least one additional subunit.

16. The composition of claim 15, wherein the first subunit and the at least one additional subunits are combined into an encapsulation medium.

17. The composition of claim 16, wherein the encapsulation medium is a capsule, a soft gel cap, a gel cap, a coating, or any combination thereof.

18. The method of claim 17, wherein the coating comprises a membrane, a film, a wax, a varnish, a glaze, a polymer coating, a sugar coating, a polysaccharide based coating, an enteric coating, or a combination thereof.

19. The composition of claim 1, wherein the therapeutically effective amount of 3-phenylsulfonyl-8-piperazinyl-1yl-quinoline or pharmaceutically acceptable salts, hydrates, polymorphs, or solvates thereof is from about 0.001 mg to about 1,000 mg, about 0.001 mg to about 200 mg, about 0.001 mg to about 175 mg, or 0.001 mg to about 70 mg.

20. The composition of claim 1, wherein the therapeutically effective amount of 3-phenylsulfonyl-8-piperazinyl-1yl-quinoline or pharmaceutically acceptable salts, hydrates, polymorphs, or solvates thereof is about 15 mg, about 35 mg, or about 70 mg.

21. The composition of claim 1, wherein the therapeutically effective amount of 3-phenylsulfonyl-8-piperazinyl-1yl-quinoline or pharmaceutically acceptable salts, hydrates, polymorphs, or solvates thereof is an amount selected from the group consisting of an amount of 3-phenylsulfonyl-8-piperazinyl-1yl-quinoline that may cause convulsions in a subject to which it is administered; an amount that would be expected to exceed the maximum tolerated dose for the subject to which it is administered; an amount associated with systemic exposures characterized by an AUCₜₐᵤ₋ₛₛ of about 8.2 μg·h/ml, a Cₘₐₓ of about 0.26 μg/ml; or a combination thereof an mount associated with systemic exposures characterized by an AUC, Cₘₐₓ, or combinations thereof, that are about 2 to about 3 times higher than the mean clinical exposure achieved at the proposed clinical dose for monotherapy with 3-phenylsulfonyl-8-piperazinyl-1yl-quinoline (i.e. mean AUCₜₐᵤ₋ₛₛ of about 3.2 μg·h/ml and Cₘₐₓ of about 0.180 μg/ml), an amount associated with a recorded systemic clinical exposure that is greater than the highest recorded systemic clinical exposure (AUC_{0-∞} of about 9.25 μg·h/ml and Cₘₐₓ of about 0.293 μg/ml), an amount of 3-phenylsulfonyl-8-piperazinyl-1yl-quinoline that is greater than about 10 mg/kg/day, an amount of 3-phenylsulfonyl-8-piperazinyl-1 yl-quinoline that is greater than 15 mg/day, a dose of 3-phenylsulfonyl-8-piperazinyl-1yl-quinoline that is greater than about 35 mg/day or any combination thereof.

22. The composition of claim 1, wherein the at least one additional therapeutic agent is selected from the group consisting of an acetylcholinesterase inhibitor, an NMDA receptor antagonist, a 5HT_{2A} inverse agonist or any combination thereof.

23. The composition of claim 22, wherein the acetylcholinesterase inhibitor is selected from the group consisting of donepezil, rivastigmine, galantamine, or pharmaceutically acceptable salts, hydrates, polymorphs, or solvates thereof.

24. The composition of claim 23, wherein the acetylcholinesterase inhibitor is donepezil or pharmaceutically acceptable salts, hydrates, polymorphs, or solvates thereof.

25. The composition of claim 24, wherein the therapeutically effective amount of donepezil or pharmaceutically acceptable salts, hydrates, polymorphs, or solvates thereof is configured for immediate release, extended release, delayed release, or any combination thereof.

26. The composition of claim 24, wherein the therapeutically effective amount of donepezil or pharmaceutically acceptable salts, hydrates, polymorphs, or solvates thereof is from about 0.001 mg to about 1,000 mg, or about 0.001 mg to about 30 mg.

27. The composition of claim 24, wherein the therapeutically effective amount of donepezil or pharmaceutically acceptable salts, hydrates, polymorphs, or solvates thereof is about 5 mg, 10 mg, or 23 mg.

28. The composition of claim 23, wherein the acetylcholinesterase inhibitor is rivastigmine or pharmaceutically acceptable salts, hydrates, polymorphs, or solvates thereof.

29. The composition of claim 28, wherein the therapeutically effective amount of rivastigmine or pharmaceutically acceptable salts, hydrates, polymorphs, or solvates thereof is from about 0.001 mg to about 1,000 mg, or about 0.001 mg to about 15 mg.

30. The composition of claim 28, wherein the therapeutically effective amount of rivastigmine or pharmaceutically acceptable salts, hydrates, polymorphs, or solvates thereof is about 1.5 mg, about 3 mg, about 4.5 mg, about 6 mg, about 9 mg, about 9.5 mg, about 12 mg, or about 13.3 mg.

31. The composition of claim 28, wherein the therapeutically effective amount of rivastigmine or pharmaceutically acceptable salts, hydrates, polymorphs, or solvates thereof is configured for immediate release, for extended release, delayed release, or any combination thereof.

32. The composition of claim 23, wherein the acetylcholinesterase inhibitor is galantamine or pharmaceutically acceptable salts, hydrates, polymorphs, or solvates thereof.

33. The composition of claim 22, wherein the therapeutically effective amount of galantamine or pharmaceutically acceptable salts, hydrates, polymorphs, or solvates thereof is configured for immediate release, extended release, delayed release, or any combination thereof.

34. The composition of claim 22, wherein the therapeutically effective amount of galantamine or pharmaceutically acceptable salts, hydrates, polymorphs, or solvates thereof is from about 0.001 mg to about 1,000 mg, or about 0.001 mg to about 30 mg.

35. The composition of claim 22, wherein the therapeutically effective amount of galantamine or pharmaceutically acceptable salts, hydrates, polymorphs, or solvates thereof is about 4 mg, about 8 mg, about 12 mg, about 16 mg, or about 24 mg.

36. The composition of claim 22, wherein NMDA receptor antagonist is selected from the group consisting of memantine, amantadine, ketamine, or pharmaceutically acceptable salts, hydrates, polymorphs, or solvates thereof.

37. The composition of claim 36, wherein the NMDA receptor antagonist is memantine or pharmaceutically acceptable salts, hydrates, polymorphs, or solvates thereof.

38. The composition of claim 37, wherein the therapeutically effective amount of memantine or pharmaceutically acceptable salts, hydrates, polymorphs, or solvates thereof is configured for immediate release, extended release, delayed release, or any combination thereof.

39. The composition of claim 37, wherein the therapeutically effective amount of memantine or pharmaceutically acceptable salts, hydrates, polymorphs, or solvates thereof is from about 0.001 mg to about 1,000 mg, or about 0.001 mg to about 30 mg.

40. The composition of claim 37, wherein the therapeutically effective amount of memantine or pharmaceutically acceptable salts, hydrates, polymorphs, or solvates thereof is about 5 mg, about 7 mg, about 10 mg, about 14 mg, about 20 mg, about 21 mg, or about 28 mg.

41. The composition of claim 37, wherein the therapeutically effective amount of memantine or pharmaceutically acceptable salts, hydrates, polymorphs, or solvates thereof is configured for extended release, delayed release or any combination thereof.

42. The composition of claim 22, wherein the NMDA receptor antagonist is amantadine or pharmaceutically acceptable salts, hydrates, polymorphs, or solvates thereof.

43. The composition of claim 42, wherein the therapeutically effective amount of amantadine or pharmaceutically acceptable salts, hydrates, polymorphs, or solvates thereof is configured for immediate release, extended release, delayed release, or any combination thereof.

44. The composition of claim 42, wherein the therapeutically effective amount of amantadine or pharmaceutically acceptable salts, hydrates, polymorphs, or solvates thereof is from about 0.001 mg to about 1,000 mg, or about 0.001 mg to about 500 mg.

45. The composition of claim 42, wherein the therapeutically effective amount of amantadine or pharmaceutically acceptable salts, hydrates, polymorphs, or solvates thereof is from about 100 mg to about 400 mg.

46. The composition of claim 42, wherein the therapeutically effective amount of amantadine or pharmaceutically acceptable salts, hydrates, polymorphs, or solvates thereof is about 100 mg, 200 mg, 300 mg or about 400 mg.

47. The composition of claim 22, wherein the 5-HT_{2A} inverse agonist is nelotanserin, pimavanserin, pruvanserin, eplivanserin, volinanserin, glemanserin, ketanserin, ritanserin, clozapine, or pharmaceutically acceptable salts, hydrates, polymorphs, or solvates thereof.

48. The composition of claim 47, wherein the nelotanserin or pharmaceutically acceptable salts, hydrates, polymorphs, or solvates thereof comprises Form I of 1-[3-(4-bromo-2-methyl-2H-pyrazol-3-yl)-4-methoxy-phenyl]-3-(2,4-difluoro-phenyl)-urea, Form II of 1-[3-[4-bromo-2-methyl-2H-pyrazol-3-yl)-4-methoxy-phenyl]-3-(2,4-difluoro-phenyl)-urea or a combination thereof.

49. The composition of claim 47, wherein the therapeutically effective amount of nelotanserin or pharmaceutically acceptable salts, hydrates, polymorphs, or solvates thereof is configured for immediate release, extended release, delayed release, or any combination thereof.

50. The composition of claim 47, wherein the therapeutically effective amount of nelotanserin or pharmaceutically acceptable salts, hydrates, polymorphs, or solvates thereof is from about 0.001 mg to about 1,000 mg, or about 0.001 mg to about 100 mg.

51. The composition of claim 47, wherein the therapeutically effective amount of nelotanserin or pharmaceutically acceptable salts, hydrates, polymorphs, or solvates thereof is about 20 mg, about 40 mg, or about 80 mg.

52. The composition of claim 1, wherein the at least one pharmaceutically acceptable excipient is selected from the group consisting of microcrystalline cellulose, mannitol, sodium starch glycolate, hydroxypropyl methylcellulose, purified water, magnesium stearate, croscarmellose sodium, a glue, and any combination thereof.

53. A method of treating a neurodegenerative disease in a subject in need thereof comprising administering to said patient a therapeutically effective amount of the composition of claim 1.

54. The method of claim 53, wherein the neurodegenerative disease is selected from Alzheimer's disease (including mild or early-stage Alzheimer's disease, mild to moderate Alzheimer's disease, moderate or mid-stage Alzheimer's disease, moderate to severe Alzheimer's disease, moderately severe Alzheimer's disease, severe Alzheimer's disease, Alzheimer's disease with Lewy bodies, (AD)), Parkinson's disease (including Parkinson's disease chemically induced by exposure to environmental agents such as pesticides, insecticides, or herbicides and/or metals such as manganese, aluminum, cadmium, copper, or zinc, SNCA gene-linked Parkinson's disease, sporadic or idiopathic Parkinson's disease, or Parkin- or LRRK2-linked Parkinson's disease (PD)), autosomal-dominant Parkinson's disease, Diffuse Lewy Body Disease (DLBD) also known as Dementia with Lewy Bodies (DLB), Pure Autonomic Failure, Lewy body dysphagia, Incidental LBD, Inherited LBD (e.g., mutations of the alpha-synuclein gene, PARK3 and PARK4), multiple system atrophy (including Olivopontocerebellar Atrophy, Striatonigral Degeneration, Shy-Drager Syndrome (MSA)), combined Alzheimer's and Parkinson disease and/or MSA, Huntington's disease, synucleinopathies, disorders or conditions characterized by the presence of Lewy bodies, multiple sclerosis, Amyotrophic lateral sclerosis (ALS) dementia (including vascular dementia, Lewy body dementia, Parkinson's dementia, frontotemporal dementia), Down syndrome, Psychosis (including agitation caused by a neurodegenerative disease or associated with dopaminergic therapy such as but not limited to Parkinson's disease psychosis, Alzheimer's disease psychosis, Lewy body dementia psychosis), dyskinesia (including agitation caused by a neurodegenerative disease or associated with dopaminergic therapy), agitation (including agitation caused by a neurodegenerative disease or associated with dopaminergic therapy), conditions associated with dopaminergic therapy (including dystonia, myoclonus, or tremor), synucleinopathies, diseases, disorders or conditions associated with abnormal expression, stability, activities and/or cellular processing of α-synuclein, diseases, disorders or conditions characterized by the presence of Lewy bodies, and combinations thereof.
